(19) United States
(12) Patent Application Publication
(10) Pub. No.: US 2022/0332970 A1
(43) Pub. Date: Oct. 20, 2022

(54) PHOTOSENSITIVE COMPOSITION, FILM, OPTICAL FILTER, SOLID-STATE IMAGING ELEMENT, AND IMAGE DISPLAY DEVICE

(71) Applicant: FUJIFILM Corporation, Tokyo (JP)

(72) Inventors: Kazuya OOTA, Haibara-gun (JP); Yasuhiro SAWAMURA, Haibara-gun (JP); Akio MIZUNO, Haibara-gun (JP); Junichi ITO, Haibara-gun (JP)

(73) Assignee: FUJIFILM Corporation, Tokyo (JP)

(21) Appl. No.: 17/846,340

(22) Filed: Jun. 22, 2022

Related U.S. Application Data
(63) Continuation of application No. PCT/JP2020/046845, filed on Dec. 16, 2020.

(30) Foreign Application Priority Data
Dec. 25, 2019 (JP) 2019-233637

(51) Int. Cl. C09D 133/12; C08K 3/013; C09D 7/41

(52) U.S. Cl. CPC C09D 133/12; C08K 3/013; C09D 7/41

(57) ABSTRACT

Provided are a photosensitive composition including a coloring material A including a pigment, a pigment derivative B, and a dispersant C, in which the pigment derivative B includes a pigment derivative B1 in which a maximum value of a molar absorption coefficient in a wavelength range of 400 to 700 nm is 3000 L·mol⁻¹·cm⁻¹ or less, the dispersant C includes a dispersant C1 having an ethylenically unsaturated bond-containing group, and a total content of the coloring material A and the pigment derivative B in a total solid content of the photosensitive composition is 50% by mass or more; a film formed of the photosensitive composition; an optical filter, a solid-state imaging element; and an image display device.

### CROSS-REFERENCE TO RELATED APPLICATIONS

This application is a Continuation of PCT International Application No. PCT/JP2020/046845 (led on Dec. 16, 2020, which claims priority under 35 U.S.C § 119(a) to Japanese Patent Application No. 2019-233637 filed on Dec. 25, 2019. Each of the above application(s) is hereby expressly incorporated by reference, in its entirety, into the present application.

### BACKGROUND OF THE INVENTION

### 1. Field of the Invention

The present invention relates to a photosensitive composition including a pigment. The present invention further relates to a film formed of the photosensitive composition, an optical filter, and a solid-state imaging element.

### 2. Description of the Related Art

In recent years, as a digital camera, a mobile phone with a camera, and the like have been further spreading, there has been a greatly increasing demand for a solid-state imaging element such as a charge coupled device (CCD) image sensor. A color filter has been used as a key device in a display or an optical element.

The color filter is manufactured by using a photosensitive composition including a coloring material. In addition, in a case where a pigment is used as the coloring material, the pigment is generally dispersed in the photosensitive composition by using a pigment derivative, a dispersant, or the like.

JP2019-133154A discloses an invention relating to a photosensitive coloring composition including a pigment, a predetermined coloring agent derivative having a triazine structure, a predetermined resin type dispersant, a polymerizable compound, a photopolymerization initiator, a resin binder, and a solvent.

### SUMMARY OF THE INVENTION

In recent years, there has been a strong demand for miniaturization and film-thinning in a solid-state imaging element. Therefore, in recent years, it has been desired to further reduce a thickness of a film including a pigment, such as a color filter, used in the solid-state imaging element. In order to achieve a thin film while maintaining desired spectral performance, it is necessary to increase a concentration of the pigment in a photosensitive composition used for film formation.

On the other hand, in a case of forming a pattern (pixel) using the photosensitive composition, the photosensitive composition may be exposed in a patterned manner and then immediately developed to remove a non-exposed portion, or the photosensitive composition may be exposed and then left for a long time and developed. However, according to the studies by the present inventor, it has been found that, as a concentration of the coloring material in the photosensitive composition is increased, a pattern line width tends to vary due to the leaving.

In addition, in a case of forming a film using the photosensitive composition having a high concentration of the coloring material, the coloring material and the like tend to aggregate in the film over time and defects tend to occur. In particular, in a case where the film is left in a high humidity environment for a long time, the coloring material tends to aggregate in the film.

Therefore, an object of the present invention is to provide a photosensitive composition that a film which has excellent pattern line width stability after leaving and in which generation of defects over time is suppressed can be formed. Another object of the present invention is to provide a film formed of the photosensitive composition, an optical filter, a solid-state imaging element, and an image display device.

According to the studies conducted by the present inventor, it has been found that the above-described object can be achieved by adopting the following configuration, thereby leading to the completion of the present invention. Therefore, the present invention provides the following.

<1> A photosensitive composition comprising:

a coloring material A including a pigment;

a pigment derivative B; and

a dispersant C,

in which the pigment derivative B includes a pigment derivative B1 in which a maximum value of a molar absorption coefficient in a wavelength range of 400 to 700 nm is 3000 L·mol⁻¹·cm⁻¹ or less,

the dispersant C includes a dispersant C1 having an ethylenically unsaturated bond-containing group, and

a total content of the coloring material A and the pigment derivative B in a total solid content of the photosensitive composition is 50% by mass or more.

<2> The photosensitive composition according to <1>,

in which the pigment derivative B1 is a compound having a triazine ring.

<3> The photosensitive composition according to <1> or <2>,

in which the pigment derivative B1 is a compound including a group represented by Formula (A1),

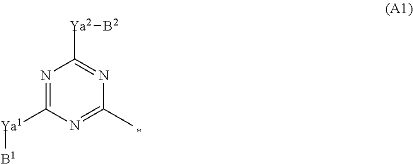

in the formula, * represents a bonding site,

Ya¹ and Ya² each independently represent —N(Ra¹)- or —O—, in which Ra¹ represents a hydrogen atom, an alkyl group, an alkenyl group, an alkynyl group, or an aryl group, and

B¹ and B² each independently represent a hydrogen atom or a substituent.

<4> The photosensitive composition according to any one of <1> to <3>,

in which the pigment derivative B includes a pigment derivative B2 in which a maximum value of a molar absorption coefficient in a wavelength range of 400 to 700 nm is more than 3000 L·mol⁻¹·cm⁻¹, and

the pigment derivative B2 is contained in an amount of 10 to 100 parts by mass with respect to 100 parts by mass of the pigment derivative B1.

<5> The photosensitive composition according to any one of <1> to <4>,

in which an ethylenically unsaturated bond-containing group value of the dispersant C1 is 0.01 to 2.0 mmol/g.

<6> The photosensitive composition according to any one of <1> to <5>,

in which the dispersant C contains 30% to 100% by mass of the dispersant C1.

<7> The photosensitive composition according to any one of <1> to <6>,

in which the pigment derivative B is contained in an amount of 3 to 30 parts by mass with respect to 100 parts by mass of a total of the coloring material A and the pigment derivative B.

<8> The photosensitive composition according to any one of <1> to <7>,

in which the dispersant C is contained in an amount of 50 to 1500 parts by mass with respect to 100 parts by mass of the pigment derivative B.

<9> The photosensitive composition according to any one of <1> to <8>,

in which the coloring material A includes a dye.

<10> The photosensitive composition according to any one of <1> to <9>, further comprising:

a photopolymerization initiator.

<11> The photosensitive composition according to any one of <1> to <10>,

in which the photosensitive composition is a photosensitive composition for forming a cyan or magenta pixel.

<12> A film obtained from the photosensitive composition according to any one of <1> to <11>.

<13> An optical filter comprising:

the film according to <12>.

<14> A solid-state imaging element comprising:

the film according to <12>.

<15> An image display device comprising:

the film according to <12>.

According to the present invention, it is possible to provide a photosensitive composition that a film which has excellent pattern line width stability after leaving and in which generation of defects over time is suppressed can be formed. It is also possible is to provide a film formed of the photosensitive composition, an optical filter, a solid-state imaging element, and an image display device.

### DESCRIPTION OF THE PREFERRED EMBODIMENTS

Hereinafter, the details of the present invention will be described.

In the present specification, “to” is used to refer to a meaning including numerical values denoted before and after “to” as a lower limit value and an upper limit value.

In the present specification, unless specified as a substituted group or as an unsubstituted group, a group (atomic group) denotes not only a group (atomic group) having no substituent but also a group (atomic group) having a substituent. For example, an “alkyl group” includes not only an alkyl group having no substituent (unsubstituted alkyl group), but also an alkyl group having a substituent (substituted alkyl group).

In the present specification, unless specified otherwise, “exposure” denotes not only exposure using light but also drawing using a corpuscular beam such as an electron beam or an ion beam. In addition, examples of light used for the exposure include actinic rays or radiation such as a bright line spectrum of a mercury lamp, far ultraviolet rays typified by an excimer laser, extreme ultraviolet rays (EUV light), X-rays, or electron beams.

In the present specification, “(meth)acrylate” denotes either or both of acrylate and methacrylate, “(meth)acryl” denotes either or both of acryl and methacryl, and “(meth)acryloyl” denotes either or both of acryloyl and methacryloyl.

In the present specification, in a structural formula, Me represents a methyl group, Et represents an ethyl group, Bu represents a butyl group, Pr represents a propyl group, and Ph represents a phenyl group.

In the present specification, a weight-average molecular weight and a number-average molecular weight are values in terms of polystyrene through measurement by a gel permeation chromatography (GPC) method.

In the present specification, near infrared rays denote light having a wavelength in a range of 700 to 2500 nm.

In the present specification, a total solid content denotes the total mass of all the components of the composition excluding a solvent.

In the present specification, a pigment means a compound which is hardly dissolved in a solvent. For example, as the pigment, both of the solubility in 100 g of water at 23° C. and 100 g of propylene glycol monomethyl ether acetate at 23° C. is preferably 0.1 g or less and more preferably 0.01 g or less.

In the present specification, the term “step” is not only an independent step, but also includes a step which is not clearly distinguished from other steps in a case where an intended action of the step is obtained.

<Photosensitive Composition>

A photosensitive composition according to an embodiment of the present invention is a photosensitive composition including a coloring material A including a pigment, a pigment derivative B, and a dispersant C, in which the pigment derivative B includes a pigment derivative B1 in which a maximum value of a molar absorption coefficient in a wavelength range of 400 to 700 nm is 3000 L·mol⁻¹·cm⁻¹ or less, the dispersant C includes a dispersant C1 having an ethylenically unsaturated bond-containing group, and a total content of the coloring material A and the pigment derivative B in a total solid content of the photosensitive composition is 50% by mass or more.

Since the photosensitive composition according to the embodiment of the present invention contains the pigment derivative B1 and the dispersant C1 having an ethylenically unsaturated bond-containing group, even in a case where the total content of the coloring material A and the pigment derivative B in the total solid content described above is 50% by mass or more, a film which has excellent pattern line width stability after leaving and in which generation of defects over time is suppressed can be formed. The reason for obtaining such an effect is presumed as follows. That is, since the pigment derivative B1 has a small molar absorption coefficient at a wavelength of 400 to 700 nm, in forming a photosensitive composition layer on a support using the photosensitive composition and exposing the photosensitive composition layer in a patterned manner, it is presumed that light can transmitted to a deep portion (support side) of the photosensitive composition layer by exposure. In addition, in the photosensitive composition according to the embodiment of the present invention, since the dispersant C1 having an ethylenically unsaturated bond-containing group is used as the dispersant, it is presumed that a crosslinking reaction of the dispersant C1 existing in the vicinity of the coloring material can be efficiently promoted by the exposure, and the film in the vicinity of the coloring material can be sufficiently cured. Therefore, even in a case where a development treatment is performed after exposure and leaving for a long time, it is presumed that it is possible to suppress the occurrence of aggregation of the coloring material in an exposed portion during the leaving, and as a result, variation of a pattern line width due to the leaving can be suppressed. In addition, since the film can be sufficiently cured by the exposure, it is presumed that it is possible to suppress the aggregation of the coloring material and the like in the film over time, and a film in which the generation of defects over time is suppressed can be formed.

The photosensitive composition according to the embodiment of the present invention is preferably used as a photosensitive composition for an optical filter. Examples of the optical filter include a color filter, a near infrared transmitting filter, and a near infrared cut filter, and a color filter is preferable.

Examples of the color filter include a filter having a colored pixel which transmits light having a specific wavelength, and a filter having at least one colored pixel selected from a red pixel, a blue pixel, a green pixel, a yellow pixel, a cyan pixel, or a magenta pixel is preferable. The colored pixel of the color filter can be formed using a photosensitive composition containing a chromatic coloring material.

Examples of the near infrared cut filter include a filter having a maximal absorption wavelength in a wavelength range of 700 to 1800 nm. As the near infrared cut filter, a filter having a maximal absorption wavelength in a wavelength range of 700 to 1300 nm is preferable, and a filter having a maximal absorption wavelength in a wavelength range of 700 to 1000 nm is more preferable. In addition, in the near infrared cut filter, a transmittance of in the entire wavelength range of 400 to 650 nm is preferably 70% or more, more preferably 80% or more, and still more preferably 90% or more. In addition, the transmittance at at least one point in a wavelength range of 700 to 1800 nm is preferably 20% or less. In addition, in the near infrared cut filter, absorbance Aₘₐₓ/absorbance A₅₅₀, which is a ratio of an absorbance Amax at a maximal absorption wavelength to an absorbance A550 at a wavelength of 550 nm, is preferably 20 to 500, more preferably 50 to 500, still more preferably 70 to 450, and particularly preferably 100 to 400. The near infrared cut filter can be formed using a photosensitive composition containing a near infrared absorbing coloring material.

The near infrared transmitting filter is a filter which transmits at least a part of near infrared rays. The near infrared transmitting filter may be a filter (transparent film) which transmits both visible light and near infrared ray, or may be a filter which shields at least a part of visible light and transmits at least a part of near infrared rays. Preferred examples of the near infrared transmitting filter include filters satisfying spectral characteristics in which the maximum value of a transmittance in a wavelength range of 400 to 640 nm is 20% or less (preferably 15% or less and more preferably 10% or less) and the minimum value of a transmittance in a wavelength range of 1100 to 1300 nm is 70% or more (preferably 75% or more and more preferably 80% or more). The near infrared transmitting filter is preferably a filter which satisfies any one of the following spectral characteristics (1) to (4).

(1): filter in which the maximum value of a transmittance in a wavelength range of 400 to 640 nm is 20% or less (preferably 15% or less and more preferably 10% or less) and the minimum value of a transmittance in a wavelength range of 800 to 1300 nm is 70% or more (preferably 75% or more and more preferably 80% or more).

(2): filter in which the maximum value of a transmittance in a wavelength range of 400 to 750 nm is 20% or less (preferably 15% or less and more preferably 10% or less) and the minimum value of a transmittance in a wavelength range of 900 to 1300 nm is 70% or more (preferably 75% or more and more preferably 80% or more).

(3): filter in which the maximum value of a transmittance in a wavelength range of 400 to 830 nm is 20% or less (preferably 15% or less and more preferably 10% or less) and the minimum value of a transmittance in a wavelength range of 1000 to 1300 nm is 70% or more (preferably 75% or more and more preferably 80% or more).

(4): filter in which the maximum value of a transmittance in a wavelength range of 400 to 950 nm is 20% or less (preferably 15% or less and more preferably 10% or less) and the minimum value of a transmittance in a wavelength range of 1100 to 1300 nm is 70% or more (preferably 75% or more and more preferably 80% or more).

The photosensitive composition according to the embodiment of the present invention can be preferably used as a photosensitive composition for forming a cyan or magenta pixel. Since the cyan pixel or the magenta pixel has high light transmittance on a shortwave side in the visible light range, in a case where the coloring material aggregates in the film, the influence of variation in spectral characteristics due to the aggregation of the coloring material tends to be large. However, according to the photosensitive composition according to the embodiment of the present invention, since the aggregation of the coloring material in the film can be effectively suppressed, the effects of the present invention are remarkably exhibited in a case where the pixels of these hues are formed by using the photosensitive composition according to the embodiment of the present invention. Further, by using a pigment derivative having high transparency in the visible region, it is possible to manufacture a cyan pixel or magenta pixel having high transmittance, and it is expected that sensitivity of a sensor can be improved.

A concentration of solid contents of the photosensitive composition according to the embodiment of the present invention is preferably 5% to 30% by mass. The lower limit is preferably 7.5% by mass or more and more preferably 10% by mass or more. The upper limit is preferably 25% by mass or less, more preferably 20% by mass or less, and still more preferably 15% by mass or less.

Hereinafter, each of the components used in the photosensitive composition according to the embodiment of the present invention will be described.

<<Coloring Material A>>

The photosensitive composition according to the embodiment of the present invention contains a coloring material A including a pigment.

[Pigment]

Examples of the pigment include a white pigment, a black pigment, a chromatic pigment, and a near infrared absorbing pigment. In the present specification, the white pigment includes not only a pure white pigment but also a bright gray (for example, grayish-white, light gray, and the like) pigment close to white. In addition, the pigment may be an inorganic pigment or an organic pigment, but from the viewpoint that dispersion stability is more easily improved, an organic pigment is preferable. In addition, as the pigment, a pigment having a maximal absorption wavelength in a wavelength range of 400 to 2000 nm is preferable, and a pigment having a maximal absorption wavelength in a wavelength range of 400 to 700 nm is more preferable. In addition, in a case of using a pigment (preferably a chromatic pigment) having a maximal absorption wavelength in a wavelength range of 400 to 700 nm, the photosensitive composition according to the embodiment of the present invention can be preferably used as a photosensitive composition for forming a colored pixel in a color filter.

In a case where the photosensitive composition is used for forming a near infrared cut filter, a near infrared absorbing pigment is used as the pigment. As the near infrared absorbing pigment, one kind may be included, or two or more kinds may be included. In a case where a pixel for a near infrared transmitting filter is formed by using the photosensitive composition, as the pigment, two or more kinds of chromatic pigments are used in combination, or a black pigment is used.

An average primary particle diameter of the pigment is preferably 1 to 200 nm. The lower limit is preferably 5 nm or more and more preferably 10 nm or more. The upper limit is preferably 180 nm or less, more preferably 150 nm or less, and still more preferably 100 nm or less. In a case where the average primary particle diameter of the pigment is within the above-described range, dispersion stability of the pigment in the photosensitive composition is good. In the present invention, the primary particle diameter of the pigment can be determined from an image obtained by observing primary particles of the pigment using a transmission electron microscope. Specifically, a projected area of the primary particles of the pigment is determined, and the corresponding equivalent circle diameter is calculated as the primary particle diameter of the pigment. In addition, the average primary particle diameter in the present invention is an arithmetic average of the primary particle diameters with respect to 400 primary particles of the pigment. In addition, the primary particle of the pigment refers to a particle which is independent without aggregation.

(Chromatic Pigment)

The chromatic pigment is not particularly limited, and a known chromatic pigment can be used. Examples of the chromatic pigment include a pigment having a maximal absorption wavelength in a wavelength range of 400 to 700 nm. Examples thereof include a yellow pigment, an orange pigment, a red pigment, a green pigment, a violet pigment, and a blue pigment. Specific examples of these pigments include the following pigments.

Color Index (C. I.) Pigment Yellow 1, 2, 3, 4, 5, 6, 10, 11, 12, 13, 14, 15, 16, 17, 18, 20, 24, 31, 32, 34, 35, 35:1, 36, 36:1, 37, 37:1, 40, 42, 43, 53, 55, 60, 61, 62, 63, 65, 73, 74, 77, 81, 83, 86, 93, 94, 95, 97, 98, 100, 101, 104, 106, 108, 109, 110, 113, 114, 115, 116, 117, 118, 119, 120, 123, 125, 126, 127, 128, 129, 137, 138, 139, 147, 148, 150, 151, 152, 153, 154, 155, 156, 161, 162, 164, 166, 167, 168, 169, 170, 171, 172, 173, 174, 175, 176, 177, 179, 180, 181, 182, 185, 187, 188, 193, 194, 199, 213, 214, 215, 228, 231, 232 (methine-based), 233 (quinoline-based), 234 (aminoketone-based), 235 (aminoketone-based), 236 (aminoketone-based), and the like (all of which are yellow pigments);

C. I. Pigment Orange 2, 5, 13, 16, 17:1, 31, 34, 36, 38, 43, 46, 48, 49, 51, 52, 55, 59, 60, 61, 62, 64, 71, and 73 (all of which are orange pigments);

C. I. Pigment Red 1, 2, 3, 4, 5, 6, 7, 9, 10, 14, 17, 22, 23, 31, 38, 41, 48:1, 48:2, 48:3, 48:4, 49, 49:1, 49:2, 52:1, 52:2, 53:1, 57:1, 60:1, 63:1, 66, 67, 81:1, 81:2, 81:3, 83, 88, 90, 105, 112, 119, 122, 123, 144, 146, 149, 150, 155, 166, 168, 169, 170, 171, 172, 175, 176, 177, 178, 179, 184, 185, 187, 188, 190, 200, 202, 206, 207, 208, 209, 210, 216, 220, 224, 226, 242, 246, 254, 255, 264, 269, 270, 272, 279, 291, 294 (xanthene-based, Organo Ultramarine, Bluish Red), 295 (monoazo-based), 296 (diazo-based), 297 (aminoketone-based), and the like (all of which are red pigments);

C. I. Pigment Green 7, 10, 36, 37, 58, 59, 62, 63, 64 (phthalocyanine-based), 65 (phthalocyanine-based), 66 (phthalocyanine-based), and the like (all of which are green pigments);

C. I. Pigment Violet 1, 19, 23, 27, 32, 37, 42, 60 (triarylmethane-based), 61 (xanthene-based), and the like (all of which are violet pigments); and

C. I. Pigment Blue 1, 2, 15, 15:1, 15:2, 15:3, 15:4, 15:6, 16, 22, 29, 60, 64, 66, 79, 80, 87 (monoazo-based), 88 (methine-based), and the like (all of which are blue pigments).

In addition, a halogenated zinc phthalocyanine pigment having an average number of halogen atoms in one molecule of 10 to 14, an average number of bromine atoms in one molecule of 8 to 12, and an average number of chlorine atoms in one molecule of 2 to 5 can also be used as the green pigment. Specific examples thereof include the compounds described in WO2015/118720A. In addition, as the green pigment, a compound described in CN2010-6909027A, a phthalocyanine compound described in WO2012/102395A, which has phosphoric acid ester as a ligand, a phthalocyanine compound described in JP2019-008014A, a phthalocyanine compound described in JP2018-180023A, a compound described in JP2019-038958A, and the like can also be used.

In addition, an aluminum phthalocyanine compound having a phosphorus atom can also be used as the blue pigment. Specific examples thereof include the compounds described in paragraphs 0022 to 0030 of JP2012-247591A and paragraph 0047 of JP2011-157478A.

In addition, as the yellow pigment, compounds described in JP2017-201003A, compounds described in JP2017-197719A, compounds described in paragraph Nos. 0011 to 0062 and 0137 to 0276 of JP2017-171912A, compounds described in paragraph Nos. 0010 to 0062 and 0138 to 0295 of JP2017-171913A, compounds described in paragraph Nos. 0011 to 0062 and 0139 to 0190 of JP2017-171914A, compounds described in paragraph Nos. 0010 to 0065 and 0142 to 0222 of JP2017-171915A, quinophthalone compounds described in paragraph Nos. 0011 to 0034 of JP2013-054339A, quinophthalone compounds described in paragraph Nos. 0013 to 0058 of JP2014-026228A, isoindoline compounds described JP2018-062644A, quinophthalone compounds described in JP2018-203798A, quinophthalone compounds described in JP2018-062578A, quinophthalone compounds described in JP6432077B, quinophthalone compounds described in JP6432076B, quinophthalone compounds described in JP2018-155881A, quinophthalone compounds described in JP2018-111757A, quinophthalone compounds described in JP2018-040835A, quinophthalone compounds described in JP2017-197640A, quinophthalone compounds described in JP2016-145282A, quinophthalone compounds described in JP2014-085565A, quinophthalone compounds described in JP2014-021139A, quinophthalone compounds described in JP2013-209614A, quinophthalone compounds described in JP2013-209435A, quinophthalone compounds described in JP2013-181015A, quinophthalone compounds described in JP2013-061622A, quinophthalone compounds described in JP2013-054339A, quinophthalone compounds described in JP2013-032486A, quinophthalone compounds described in JP2012-226110A, quinophthalone compounds described in JP2008-074987A, quinophthalone compounds described in JP2008-081565A, quinophthalone compounds described in JP2008-074986A, quinophthalone compounds described in JP2008-074985A, quinophthalone compounds described in JP2008-050420A, quinophthalone compounds described in JP2008-031281A, quinophthalone compounds described in JP1973-032765A (JP-S48-032765A), quinophthalone compounds described in JP2019-008014A, a compound represented by Formula (QP1), and a compound represented by Formula (QP2) can also be used. In addition, as the yellow pigment, from the viewpoint of improving resistance, it is also preferable to use C. I. Pigment Yellow 129 or C. I. Pigment Yellow 215.

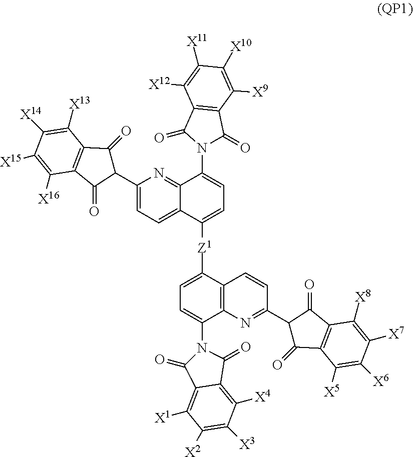

In Formula (QP2), X¹ to X¹⁶ each independently represent a hydrogen atom or a halogen atom, and Z¹ represents an alkylene group having 0 to 3 carbon atoms. Specific examples of the compound represented by Formula (QP1) include compounds described in paragraph No. 0016 of JP6443711B.

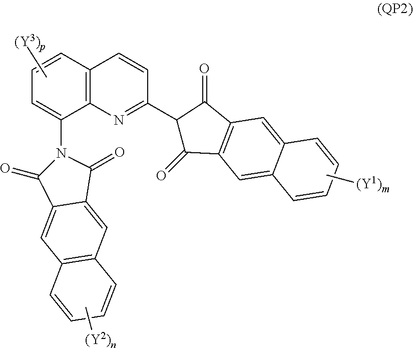

In Formula (QP2), Y¹ to Y³ each independently represent a halogen atom. n and m represent an integer of 0 to 6, and p represents an integer of 0 to 5. (n+m) is 1 or more. Specific examples of the compound represented by Formula (QP2) include compounds described in paragraph Nos. 0047 and 0048 of JP6432077B.

A diketopyrrolopyrrole pigment described in JP2017-201384A, in which the structure has at least one substituted bromine atom, a diketopyrrolopyrrole pigment described in paragraph Nos. 0016 to 0022 of JP6248838B, a red pigment described in JP6516119B, a red pigment described in JP6525101B, and the like can also be used as the red pigment. In addition, as the red pigment, a compound having a structure that an aromatic ring group in which a group bonded with an oxygen atom, a sulfur atom, or a nitrogen atom is introduced to an aromatic ring is bonded to a diketopyrrolopyrrole skeleton can be used. As the compound, a compound represented by Formula (DPP1) is preferable, and a compound represented by Formula (DPP2) is more preferable.

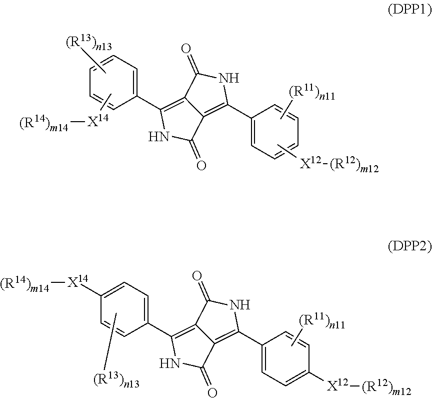

In the formulae, R¹¹ and R¹³ each independently represent a substituent, R¹² and R¹⁴ each independently represent a hydrogen atom, an alkyl group, an aryl group, or a heteroaryl group, n11 and n13 each independently represent an integer of 0 to 4, X¹² and X¹⁴ each independently represent an oxygen atom, a sulfur atom, or a nitrogen atom, in a case where X¹² is an oxygen atom or a sulfur atom, m12 represents 1, in a case where X¹² is a nitrogen atom, m12 represents 2, in a case where X¹⁴ is an oxygen atom or a sulfur atom, m14 represents 1, and in a case where X¹⁴ is a nitrogen atom, m14 represents 2. Examples of the substituent represented by R¹¹ and R¹³ include groups mentioned in the substituent T described later, and preferred specific examples thereof include an alkyl group, an aryl group, a halogen atom, an acyl group, an alkoxycarbonyl group, an aryloxycarbonyl group, a heteroaryloxycarbonyl group, an amide group, a cyano group, a nitro group, a trifluoromethyl group, a sulfoxide group, and a sulfo group.

In a case where the photosensitive composition is used as a photosensitive composition for forming a magenta pixel in a color filter, as the pigment, C. I. Pigment Red 122, C. I. Pigment Red 177, C. I. Pigment Red 202, C. I. Pigment Red 209, C. I. Pigment Violet 19, or the like is preferably used. In addition, in a case where the photosensitive composition is used as a photosensitive composition for forming a cyan pixel in a color filter, as the pigment, C. I. Pigment Green 7, C. I. Pigment Green 36, C. I. Pigment Green 58, C. I. Pigment Green 62, C. I. Pigment Green 63, C. I. Pigment Blue 15:3, C. I. Pigment Blue 15:4, C. I. Pigment Blue 16, or the like is used.

The chromatic pigment may be used in combination of two or more kinds thereof. For example, C. I. Pigment Green 7, C. I. Pigment Green 36. C. I. Pigment Yellow 139, and C. I. Pigment Yellow 185 may be combined to form green, or C. I. Pigment Green 58, C. I. Pigment Yellow 150, and C. I. Pigment Yellow 185 may be combined to form green.

In addition, in a case where the chromatic pigment is used in combination of two or more kinds thereof, the combination of two or more chromatic pigments may form black. Examples of such a combination include the following aspects (1) to (7). In a case where two or more chromatic pigments are included in the photosensitive composition and the combination of two or more chromatic pigments forms black, the photosensitive composition according to the embodiment of the present invention can be preferably used as a photosensitive composition for forming the near infrared transmitting filter.

(1) aspect in which a red pigment and a blue pigment are contained.

(2) aspect in which a red pigment, a blue pigment, and a yellow pigment are contained.

(3) aspect in which a red pigment, a blue pigment, a yellow pigment, and a violet pigment are contained.

(4) aspect in which a red pigment, a blue pigment, a yellow pigment, a violet pigment, and a green pigment are contained.

(5) aspect in which a red pigment, a blue pigment, a yellow pigment, and a green pigment are contained.

(6) aspect in which a red pigment, a blue pigment, and a green pigment are contained.

(7) aspect in which a yellow pigment and a violet pigment are contained.

(White Pigment)

Examples of the white pigment include titanium oxide, strontium titanate, barium titanate, zinc oxide, magnesium oxide, zirconium oxide, aluminum oxide, barium sulfate, silica, talc, mica, aluminum hydroxide, calcium silicate, aluminum silicate, hollow resin particles, and zinc sulfide. The white pigment is preferably particles having a titanium atom, more preferably titanium oxide. In addition, the white pigment is preferably a particle having a refractive index of 2.10 or more with respect to light having a wavelength of 589 nm. The above-mentioned refractive index is preferably 2.10 to 3.0) and more preferably 2.50 to 2.75.

In addition, as the white pigment, the titanium oxide described in “Titanium Oxide-Physical Properties and Applied Technology, written by Manabu Kiyono, pages 13 to 45, published in Jun. 25, 1991, published by Gihodo Shuppan Co., Ltd.” can also be used.

The white pigment is not limited to a compound formed of a single inorganic substance, and may be particles combined with other materials. For example, it is preferable to use a particle having a pore or other materials therein, a particle having a number of inorganic particles attached to a core particle, or a core-shell composite particle composed of a core particle formed of polymer particles and a shell layer formed of inorganic fine nanoparticles. With regard to the core-shell composite particle composed of a core particle formed of polymer particles and a shell layer formed of inorganic fine nanoparticles, reference can be made to, for example, the descriptions in paragraph Nos. 0012 to 0042 of JP2015-047520A, the contents of which are incorporated herein by reference.

As the white pigment, hollow inorganic particles can also be used. The hollow inorganic particles refer to inorganic particles having a structure with a cavity therein, and the cavity is enclosed by an outer shell. As the hollow inorganic particles, hollow inorganic particles described in JP2011-075786A, WO2013/061621A, JP2015-164881A, and the like can be used, the contents of which are incorporated herein by reference.

(Black Pigment)

The black pigment is not particularly limited, and a known black pigment can be used. Examples thereof include carbon black, titanium black, and graphite, and carbon black or titanium black is preferable and titanium black is more preferable. The titanium black is black particles containing a titanium atom, and is preferably lower titanium oxide or titanium oxynitride. The surface of the titanium black can be modified, as necessary, according to the purpose of improving dispersibility, suppressing aggregating properties, and the like. For example, the surface of the titanium black can be coated with silicon oxide, titanium oxide, germanium oxide, aluminum oxide, magnesium oxide, or zirconium oxide. In addition, a treatment with a water-repellent substance as described in JP2007-302836A can be performed. Examples of the black pigment include Color Index (C. I.) Pigment Black 1 and 7. It is preferable that the titanium black has a small primary particle diameter of the individual particles and has a small average primary particle diameter. Specifically, the average primary particle diameter thereof is preferably 10 to 45 nm. The titanium black can be used as a dispersion. Examples thereof include a dispersion which includes titanium black particles and silica particles and in which the content ratio of Si atoms to Ti atoms is adjusted to a range of 0.20 to 0.50. With regard to the dispersion, reference can be made to the description in paragraphs 0020 to 0105 of JP2012-169556A, the contents of which are incorporated herein by reference. Examples of a commercially available product of the titanium black include Titanium black 10S, 12S, 13R, 13M, 13M-C, 13R-N, 13M-T (trade name; manufactured by Mitsubishi Materials Corporation) and Tilack D (trade name; manufactured by Akokasei Co., Ltd.). In addition, perylene black (Lumogen Black FK4280 and the like) described in paragraphs 0016 to 0020 of JP2017-226821A may be used. In addition, compounds having the following structures can also be used as the black pigment.

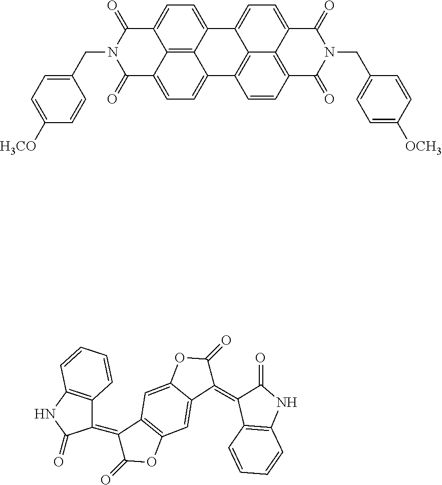 [SMILES: COc1ccc(CN2C(=O)c3ccc4c5ccc6c7c(ccc(c8ccc(c3c48)C2=O)c75)C(=O)N(Cc2ccc(OC)cc2)C6=O)cc1 ; O=C1Nc2ccccc2/C1=C1/C(=O)Oc2cc3c(cc21)OC(=O)/C3=C1\C(=O)Nc2ccccc21]

(Near Infrared Absorbing Pigment)

The near infrared absorbing pigment is preferably an organic pigment. In addition, the near infrared absorbing pigment preferably has a maximal absorption wavelength in a wavelength range of more than 700 nm and 1400 nm or less. In addition, the maximal absorption wavelength of the near infrared absorbing pigment is preferably 1200 nm or less, more preferably 1000 nm or less, and still more preferably 950 nm or less. In addition, in the near infrared absorbing pigment, A₅₅₀/Aₘₐₓ, which is a ratio of an absorbance A₅₅₀ at a wavelength of 550 nm to an absorbance Aₘₐₓ at the maximal absorption wavelength, is preferably 0.1 or less, more preferably 0.05 or less, still more preferably 0.03 or less, and particularly preferably 0.02 or less. The lower limit is not particularly limited, but for example, may be 0.0001 or more or may be 0.0005 or more. In a case where the ratio of the above-described absorbance is within the above-described range, a near infrared absorbing pigment excellent in visible transparency and near infrared rays shielding property can be obtained. In the present invention, the maximal absorption wavelength of the near infrared absorbing pigment and values of absorbance at each wavelength are values obtained from an absorption spectrum of a film formed by using a photosensitive composition including the near infrared absorbing pigment.

The near infrared absorbing pigment is not particularly limited, and examples thereof include a pyrrolopyrrole compound, a rylene compound, an oxonol compound, a squarylium compound, a cyanine compound, a croconium compound, a phthalocyanine compound, a naphthalocyanine compound, a pyrylium compound, an azurenium compound, an indigo compound, and a pyrromethene compound. Among these, at least one compound selected from a pyrrolopyrrole compound, a squarylium compound, a cyanine compound, a phthalocyanine compound, or a naphthalocyanine compound is preferable, and a pyrrolopyrrole compound or a squarylium compound is more preferable, and a pyrrolopyrrole compound is particularly preferable. Specific examples of the near infrared absorbing pigment include compounds described in Examples described later.

[Dye]

The coloring material A used in the photosensitive composition according to the embodiment of the present invention may further include a dye. In a case where the photosensitive composition contains a dye in addition to the pigment, a highly transparent color filter can be manufactured. Further, by using a pigment and a dye in combination, it is possible to provide a photosensitive composition having more excellent leaving stability. The dye is not particularly limited and a known dye can be used. The dye may be a chromatic dye or may be a near infrared absorbing dye. Examples of the chromatic dye include a pyrazoleazo compound, an anilinoazo compound, a triarylmethane compound, an anthraquinone compound, an anthrapyridone compound, a benzylidene compound, an oxonol compound, a pyrazolotriazoleazo compound, a pyridoneazo compound, a cyanine compound, a phenothiazine compound, a pyrrolopyrazoleazomethine compound, a xanthene compound, a phthalocyanine compound, a benzopyran compound, an indigo compound, and a pyrromethene compound. In addition, the thiazole compound described in JP2012-158649A, the azo compound described in JP2011-184493A, or the azo compound described in JP2011-145540A can also be used. Examples of the near infrared absorbing dye include a pyrrolopyrrole compound, a rylene compound, an oxonol compound, a squarylium compound, a cyanine compound, a croconium compound, a phthalocyanine compound, a naphthalocyanine compound, a pyrylium compound, an azurenium compound, an indigo compound, and a pyrromethene compound. In addition, as the dye, a coloring agent multimer can also be used. The coloring agent multimer has two or more coloring agent structures in one molecule, and preferably has three or more coloring agent structures in one molecule. The upper limit is particularly not limited, but may be 100 or less. A plurality of coloring agent structures included in one molecule may be the same coloring agent structure or different coloring agent structures. The weight-average molecular weight (Mw) of the coloring agent multimer is preferably 2000 to 50000. The lower limit is more preferably 3000 or more and still more preferably 6000 or more. The upper limit is more preferably 30000 or less and still more preferably 20000 or less. As the coloring agent multimer, the compounds described in JP2011-213925A, JP2013-041097A, JP2015-028144A, JP2015-030742A, JP2016-102191A, WO2016/031442A, or the like can also be used.

A content of the coloring material A in the total solid content of the photosensitive composition is preferably 45% by mass or more, more preferably 50% by mass or more, still more preferably 60% by mass or more, and even more preferably 70% by mass or more. The upper limit is preferably 90% by mass or less, more preferably 85% by mass or less, and still more preferably 80% by mass or less.

In addition, a content of the pigment in the total solid content of the photosensitive composition is preferably 45% by mass or more, more preferably 50% by mass or more, still more preferably 60% by mass or more, and even more preferably 70% by mass or more. The upper limit is preferably 90% by mass or less, more preferably 85% by mass or less, and still more preferably 80% by mass or less.

In addition, the content of the pigment in the coloring material A is preferably 1% to 100% by mass, more preferably 5% to 100% by mass, and still more preferably 10% to 100% by mass.

In a case where the coloring material A includes a dye, a content of the dye in the coloring material A is preferably 30% to 90%, by mass, more preferably 40%, to 90% by mass, and still more preferably 50% to 90% by mass.

In addition, it is also preferable that the coloring material A is substantially only the pigment. The case where the coloring material A is substantially only the pigment means that the content of the pigment in the coloring material A is 99% by mass or more, preferably 99.9% by mass or more and more preferably 100% by mass.

<<Pigment Derivative B>>

The photosensitive composition according to the embodiment of the present invention contains a pigment derivative B. The pigment derivative B used in the photosensitive composition according to the embodiment of the present invention includes a pigment derivative (hereinafter, also referred to as a pigment derivative B1) in which the maximum value of a molar absorption coefficient in a wavelength range of 400 to 700 nm is 3000 L·mol⁻¹·cm⁻¹ or less.

The maximum value of the molar absorption coefficient of the pigment derivative B1 in a wavelength range of 400 to 700 nm is preferably 1000 L·mol⁻¹·cm⁻¹ or less and more preferably 100 L·mol⁻¹·cm⁻¹. The lower limit of the maximum value of the molar absorption coefficient described above is, for example, 1 L·mol⁻¹·cm⁻¹ or more and may be 10 L·mol⁻¹·cm⁻¹ or more.

It is also preferable that the pigment derivative B1 satisfies any one of the following spectral characteristics (a) to (d).

(a) maximum value of the molar absorption coefficient in a wavelength range of more than 700 nm and 750 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

(b) maximum value of the molar absorption coefficient in a wavelength range of more than 750 nm and 800 nm or less is preferably 300) L·mol⁻¹·cm⁻¹ or less, more preferably 1000 L·mol⁻¹·cm⁻¹ or less, and still more preferably 100 L·mol⁻¹·cm⁻¹ or less.

(c) maximum value of the molar absorption coefficient in a wavelength range of more than 800 nm and 850 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

(d) maximum value of the molar absorption coefficient in a wavelength range of more than 850 nm and 900 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

The pigment derivative B1 preferably includes an aromatic ring. The aromatic ring may be an aromatic hydrocarbon ring or an aromatic heterocyclic ring. In addition, the aromatic ring may be a single ring or a fused ring. Specifically, as the aromatic ring, an aromatic ring selected from a benzene ring, a naphthalene ring, a fluorene ring, a perylene ring, an imidazole ring, a pyrazole ring, an oxazole ring, a thiazole ring, an imidazoline ring, a pyridine ring, a triazole ring, an imidazoline ring, a pyrazine ring, a pyrimidine ring, a pyridazine ring, a quinoline ring, an isoquinoline ring, a quinoxaline ring, a quinazoline ring, a benzimidazole ring, a benzopyrazole ring, a benzoxazole ring, a benzothiazole ring, a benzotriazole ring, an indole ring, an isoindole ring, a triazine ring, a pyrrole ring, a carbazole ring, a benzimidazolinone ring, a phthalimide ring, a phthalocyanine ring, an anthraquinone ring, a diketopyrrolopyrrole ring, an isoindolinone ring, an isoindoline ring, and a quinacridone ring; a fused ring including these aromatic rings; or the like is preferable. The above-described fused ring may be an aromatic ring or a non-aromatic ring as a whole, but is preferably an aromatic ring. In addition, the pigment derivative B1 may have only one aromatic ring or fused ring, but for the reason that, as the number of aromatic rings increases, pigment adsorbability is improved by π-π interaction, and it is easy to suppress the aggregation of the pigment in the film, it is preferable to have two or more of these rings. The above-described aromatic ring or fused ring may further have a substituent. Examples of the substituent include the substituent T described later.

The pigment derivative B1 preferably has a structure which easily interacts with the pigment included in the photosensitive composition or a structure similar to the pigment. According to this aspect, it is easy to more effectively suppress the aggregation of the pigment in the film. In addition, from the reason that the effects of the present invention are more easily obtained remarkably, the pigment derivative B1 preferably has an aromatic heterocyclic ring, more preferably has a nitrogen-containing aromatic heterocyclic ring, and still more preferably has a triazine ring.

It is particularly preferable that the pigment derivative B1 has a group represented by Formula (A1) including a triazine ring as the aromatic ring.

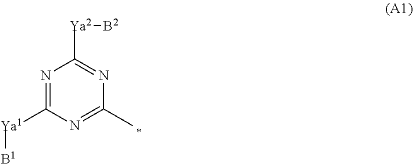

In the formula, * represents a bonding site,

Ya¹ and Ya² each independently represent —N(Ra¹)- or —O—, in which Ra¹ represents a hydrogen atom, an alkyl group, an alkenyl group, an alkynyl group, or an aryl group, and

B¹ and B² each independently represent a hydrogen atom or a substituent.

In Formula (A1), Ya¹ and Ya² each independently represent —N(Ra¹)- or —O—, and from the reason that the effects of the present invention are more easily obtained remarkably, —N(Ra¹)- is more preferable.

Ra¹ represents a hydrogen atom, an alkyl group, an alkenyl group, an alkynyl group, or an aryl group, and a hydrogen atom or an alkyl group is preferable and a hydrogen atom is more preferable.

The alkyl group represented by Ra¹ preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The alkyl group represented by Ra¹ may further have a substituent. Examples of the substituent include the substituent T described later.

The alkenyl group represented by Ra¹ preferably has 2 to 20 carbon atoms, more preferably has 2 to 12 carbon atoms, and still more preferably has 2 to 8 carbon atoms. The alkenyl group may be any of linear, branched, and cyclic forms, and is preferably linear or branched and more preferably linear. The alkenyl group represented by Ra¹ may further have a substituent. Examples of the substituent include the substituent T described later.

The alkynyl group represented by Ra¹ preferably has 2 to 40 carbon atoms, more preferably has 2 to 30 carbon atoms, and still more preferably has 2 to 25 carbon atoms. The alkynyl group may be any of linear, branched, and cyclic forms, and is preferably linear or branched and more preferably linear. The alkynyl group represented by Ra¹ may further have a substituent. Examples of the substituent include the substituent T described later.

The aryl group represented by Ra¹ preferably has 6 to 30 carbon atoms, more preferably has 6 to 20 carbon atoms, and still more preferably has 6 to 12 carbon atoms. The aryl group represented by Ra¹ may further have a substituent. Examples of the substituent include the substituent T described later.

In Formula (A1), B¹ and B² each independently represent a hydrogen atom or a substituent. Examples of the substituent include the substituent T described later, and an alkyl group, an aryl group, or a heterocyclic group is preferable, an aryl group or a heterocyclic group is more preferable, and an aryl group is still more preferable from the reason that pigment adsorbability is enhanced and storage stability of the composition is easily improved. In addition, from the reason that color unevenness can be more easily suppressed, at least one of B¹ or B² is also preferably a heterocyclic group. The heterocyclic group is preferably a nitrogen-containing heterocyclic group and more preferably a benzimidazolone group.

The alkyl group, aryl group, and heterocyclic group represented by B¹ and B² may further have a substituent. Examples of the further substituent include an alkyl group (preferably an alkyl group having 1 to 30 carbon atoms), a fluoroalkyl group (preferably a fluoroalkyl group having 1 to 30 carbon atoms), an alkenyl group (preferably an alkenyl group having 2 to 30 carbon atoms), an alkynyl group (preferably an alkynyl group having 2 to 30 carbon atoms), an aryl group (preferably an aryl group having 6 to 30 carbon atoms), an amino group (preferably an amino group having 0 to 30 carbon atoms), an alkoxy group (preferably an alkoxy group having 1 to 30 carbon atoms), an aryloxy group (preferably an aryloxy group having 6 to 30 carbon atoms), a heteroaryloxy group, an acyl group (preferably an acyl group having 1 to 30 carbon atoms), an alkoxycarbonyl group (preferably an alkoxycarbonyl group having 2 to 30 carbon atoms), an aryloxycarbonyl group (preferably an aryloxycarbonyl group having 7 to 30 carbon atoms), an acyloxy group (preferably an acyloxy group having 2 to 30 carbon atoms), an acylamino group (preferably an acylamino group having 2 to 30 carbon atoms), an alkoxycarbonylamino group (preferably an alkoxycarbonylamino group having 2 to 30 carbon atoms), an aryloxycarbonylamino group (preferably an aryloxycarbonylamino group having 7 to 30 carbon atoms), a sulfamoyl group (preferably a sulfamoyl group having 0 to 30 carbon atoms), a carbamoyl group (preferably a carbamoyl group having 1 to 30 carbon atoms), an alkylthio group (preferably an alkylthio group having 1 to 30 carbon atoms), an arylthio group (preferably an arylthio group having 6 to 30 carbon atoms), a heteroarylthio group (preferably a heteroarylthio group having 1 to 30 carbon atoms), an alkylsulfonyl group (preferably an alkylsulfonyl group having 1 to 30 carbon atoms), an arylsulfonyl group (preferably an arylsulfonyl group having 6 to 30 carbon atoms), a heteroarylsulfonyl group (preferably a heteroarylsulfonyl group having 1 to 30 carbon atoms), an alkylsulfinyl group (preferably an alkylsulfinyl group having 1 to 30 carbon atoms), an arylsulfinyl group (preferably an arylsulfinyl group having 6 to 30 carbon atoms), a heteroarylsulfinyl group (preferably a heteroarylsulfinyl group having 1 to 30 carbon atoms), a ureido group (preferably a ureido group having 1 to 30 carbon atoms), a phosphoric acid amide group (preferably a phosphoric acid amide group having 1 to 30 carbon atoms), a hydroxyl group, a carboxyl group, a sulfo group, a phosphoric acid group, a mercapto group, a halogen atom, a cyano group, an alkylsulfino group, an arylsulfino group, a hydrazino group, and an imino group. Among these, an alkyl group, a fluoroalkyl group, an alkoxy group, an amino group, a halogen atom, an alkenyl group, a hydroxyl group, an alkoxycarbonyl group, an acyloxy group, an acylamino group, or a nitro group is preferable.

It is also preferable that the alkyl group, aryl group, and heterocyclic group represented by B¹ and B² do not have the above-described further substituent.

(Substituent T)

Examples of a substituent T include a halogen atom, a cyano group, a nitro group, an alkyl group, an alkenyl group, an alkynyl group, an aryl group, a heterocyclic group, —ORt¹, —CORt¹, —COORt¹, —OCORt¹, —NRt¹Rt², —NHCORt¹, —CONRt¹Rt², —NHCONRt¹Rt², —NHCOORt¹, —SRt¹, —SO₂Rt¹, —SO₂ORt¹, —NHSO₂Rt¹, and —SO₂NRt¹Rt². Rt¹ and Rt² each independently represent a hydrogen atom, an alkyl group, an alkenyl group, an alkynyl group, an aryl group, or a heteroaryl group. Rt¹ and Rt² may be bonded to each other to form a ring.

Examples of the halogen atom include a fluorine atom, a chlorine atom, a bromine atom, and an iodine atom.

The alkyl group preferably has 1 to 30 carbon atoms, more preferably has 1 to 15 carbon atoms, and still more preferably 1 to 8 carbon atoms. The alkyl group may be any of linear, branched, and cyclic forms, and is preferably linear or branched and more preferably linear.

The alkenyl group preferably has 2 to 30 carbon atoms, more preferably has 2 to 12 carbon atoms, and particularly preferably has 2 to 8 carbon atoms. The alkenyl group may be any of linear, branched, and cyclic forms, and is preferably linear or branched and more preferably linear.

The alkynyl group preferably has 2 to 30 carbon atoms and more preferably has 2 to 25 carbon atoms. The alkynyl group may be any of linear, branched, and cyclic forms, and is preferably linear or branched and more preferably linear.

The aryl group preferably has 6 to 30 carbon atoms, more preferably has 6 to 20 carbon atoms, and still more preferably has 6 to 12 carbon atoms.

The heterocyclic group may be a single ring or a fused ring. The heterocyclic group is preferably a single ring or a fused ring having 2 to 4 fused numbers. The number of heteroatoms constituting a ring of the heterocyclic group is preferably 1 to 3. The heteroatom constituting the ring of the heterocyclic group is preferably a nitrogen atom, an oxygen atom, or a sulfur atom. The number of carbon atoms constituting the ring of the heterocyclic group is preferably 3 to 30, more preferably 3 to 18, and more preferably 3 to 12.

The alkyl group, the alkenyl group, the alkynyl group, the aryl group, and the heterocyclic group may have a substituent or may be unsubstituted. Examples of the substituent include the substituents described as the substituent T described above.

Specific examples of the aromatic ring included in the pigment derivative B1 include groups having the following structures. In the following structural formulae, Me represents a methyl group.

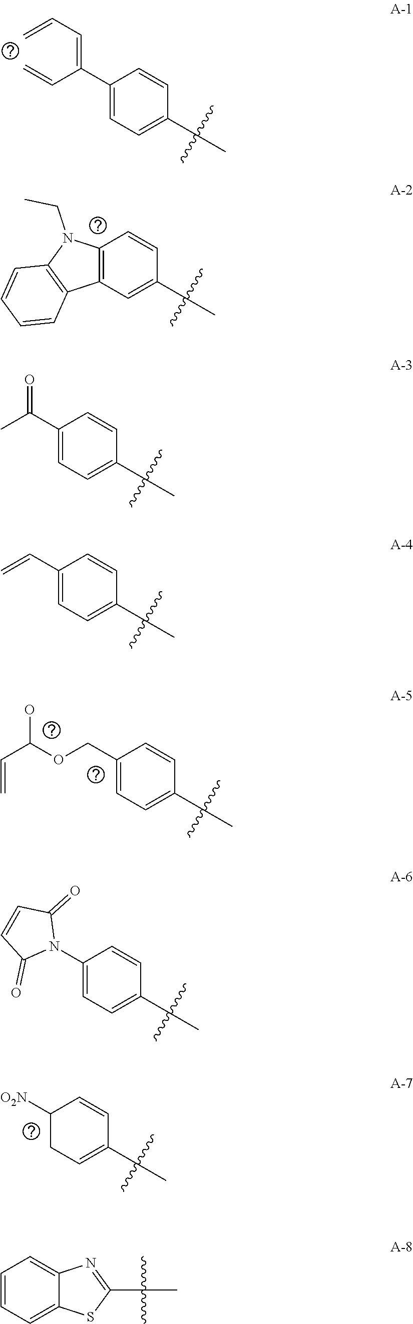

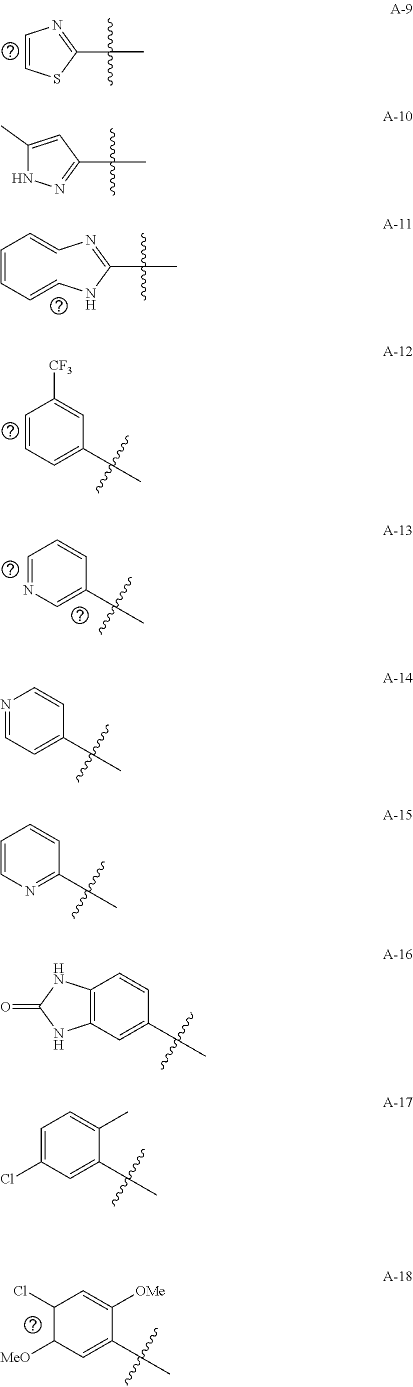

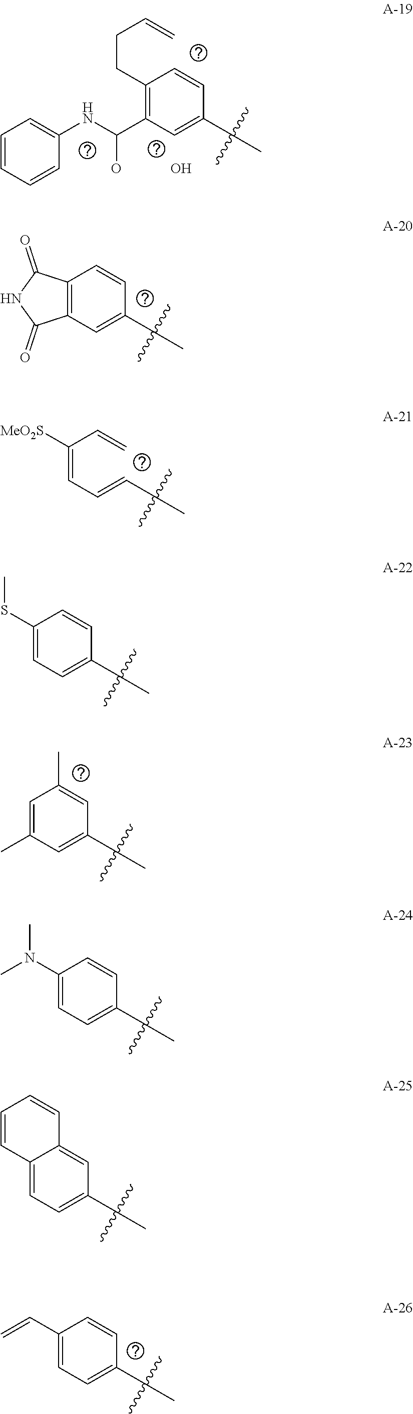

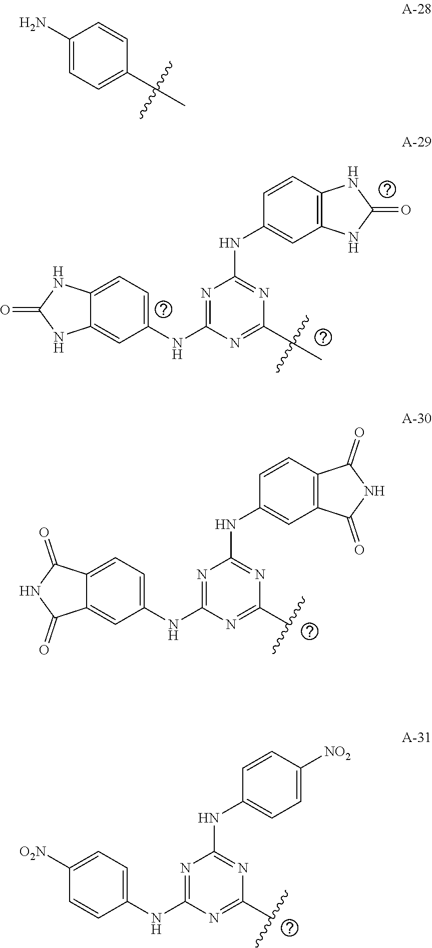 [SMILES: *c1ccc(N)cc1 ; *c1nc(Nc2ccc3[nH]c(=O)[nH]c3c2)nc(Nc2ccc3[nH]c(=O)[nH]c3c2)n1 ; *c1nc(Nc2ccc3c(c2)C(=O)NC3=O)nc(Nc2ccc3c(c2)C(=O)NC3=O)n1 ; *c1nc(Nc2ccc([N+](=O)[O-])cc2)nc(Nc2ccc([N+](=O)[O-])cc2)n1]

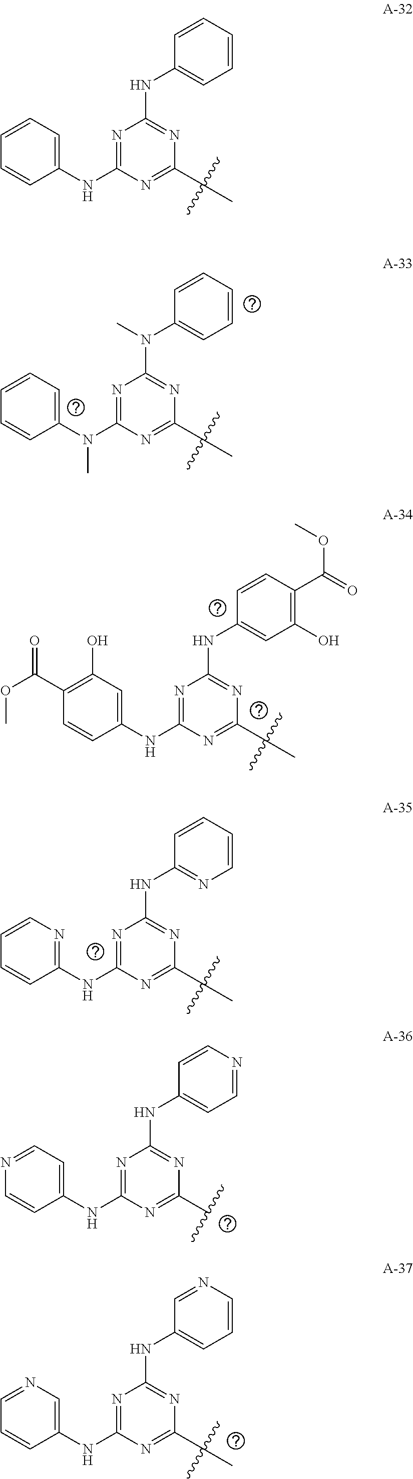 [SMILES: *c1nc(Nc2ccccc2)nc(Nc2ccccc2)n1 ; *c1nc(N(C)c2ccccc2)nc(N(C)c2ccccc2)n1 ; *c1nc(Nc2ccc(C(=O)OC)c(O)c2)nc(Nc2ccc(C(=O)OC)c(O)c2)n1 ; *c1nc(Nc2ccccn2)nc(Nc2ccccn2)n1 ; *c1nc(Nc2ccncc2)nc(Nc2ccncc2)n1 ; *c1nc(Nc2cccnc2)nc(Nc2cccnc2)n1]

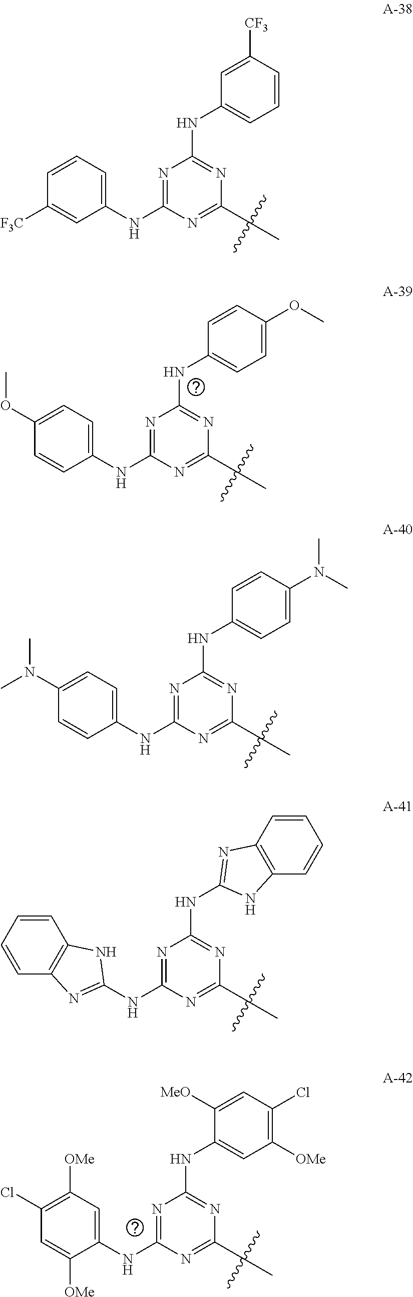

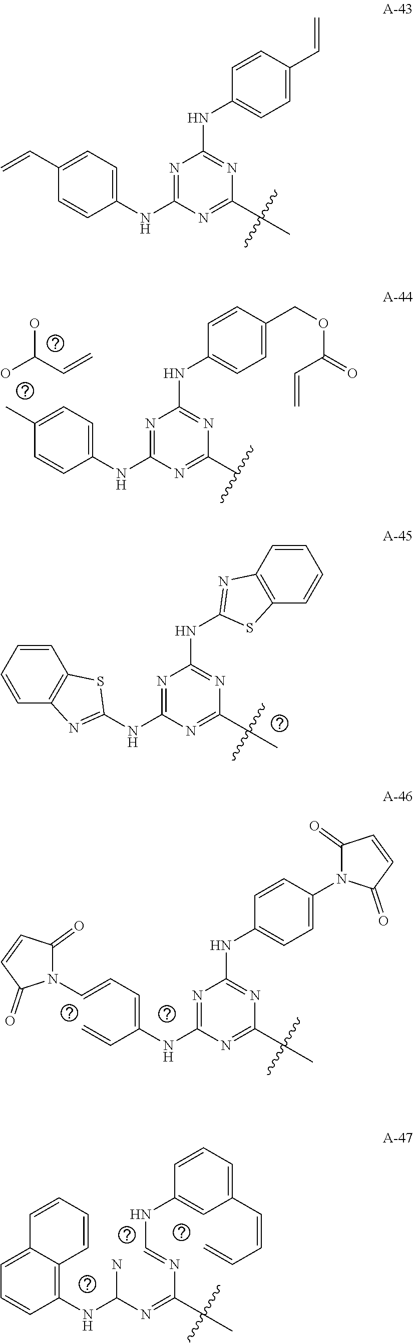

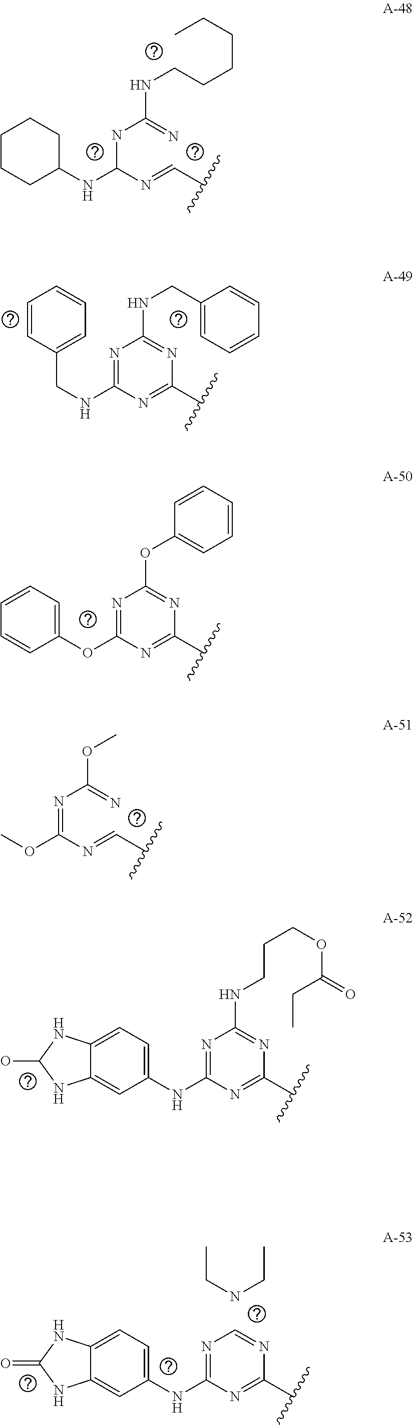

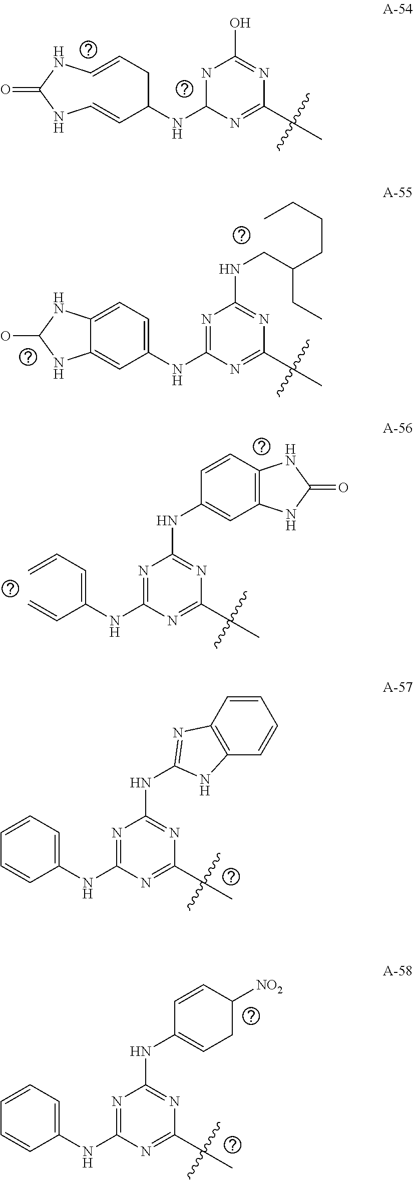

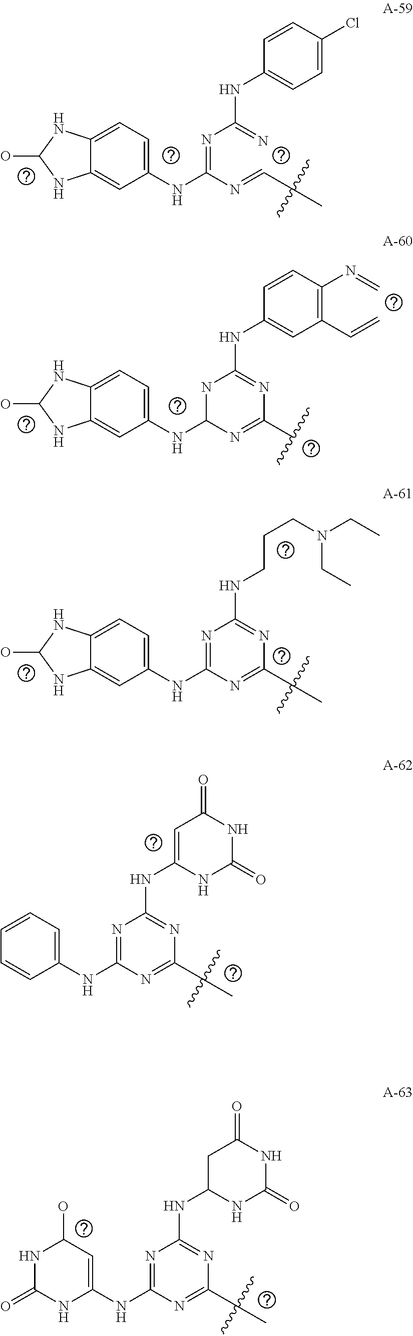 [SMILES: */C=N/C(=N\C(=N)Nc1ccc(Cl)cc1)Nc1ccc2c(c1)NC(O)N2 ; *C1=NC(Nc2ccc3c(c2)NC(O)N3)NC(Nc2ccc(N=C)c(C=C)c2)=N1 ; *c1nc(NCCCN(CC)CC)nc(Nc2ccc3c(c2)NC(O)N3)n1 ; *c1nc(Nc2ccccc2)nc(Nc2cc(=O)[nH]c(=O)[nH]2)n1 ; *c1nc(NC2=CC(O)NC(=O)N2)nc(NC2CC(=O)NC(=O)N2)n1]

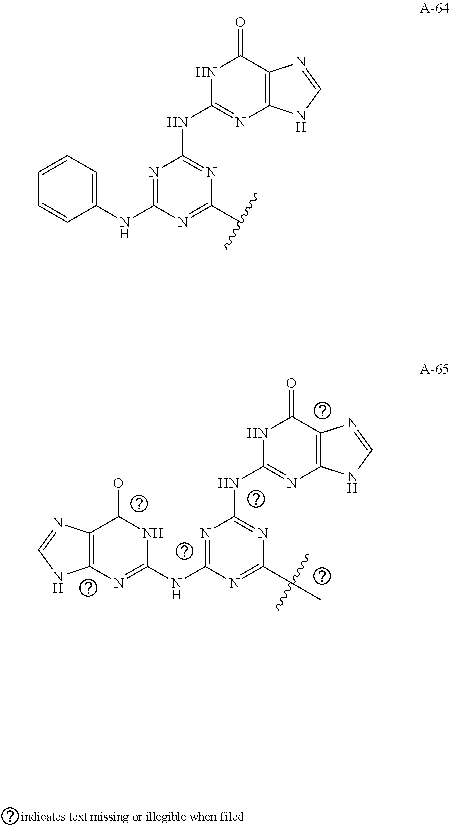 [SMILES: *c1nc(Nc2ccccc2)nc(Nc2nc3[nH]cnc3c(=O)[nH]2)n1 ; *c1nc(NC2=Nc3[nH]cnc3C(O)N2)nc(Nc2nc3[nH]cnc3c(=O)[nH]2)n1]

The pigment derivative B1 preferably includes at least one group selected from an acid group or a basic group. The acid group is preferably at least one selected from a carboxyl group, a sulfo group, a phosphoric acid group, or a salt thereof, and more preferably at least one selected from a carboxyl group, a sulfo group, or a salt thereof. Examples of an atom or atomic group constituting the salts include alkali metal ions (Li⁺, Na⁺, K⁺, and the like), alkaline earth metal ions (Ca²⁺, Mg²⁺, and the like), an ammonium ion, an imidazolium ion, a pyridinium ion, and a phosphonium ion. The basic group is preferably at least one selected from an amino group, a pyridyl group, salts thereof, a salt of an ammonium group, or a phthalimidomethyl group, more preferably at least one selected from an amino group, a salt of an amino group, or a salt of an ammonium group, and more preferably an amino group or a salt of an amino group. Examples of the amino group include —NH₂, a dialkylamino group, an alkylarylamino group, a diarylamino group, and a cyclic amino group. The dialkylamino group, alkylarylamino group, diarylamino group, and cyclic amino group may further have a substituent. Examples of the substituent include the above-described substituent T. Examples of an atom or atomic group constituting the salts include a hydroxide ion, a halogen ion, a carboxylate ion, a sulfonate ion, and a phenoxide ion.

The pigment derivative B1 is preferably a compound represented by Formula (b1).

A¹-L¹-Z¹ (b1)

In Formula (b1), A¹ represents a group including an aromatic ring,

L¹ represents a single bond or a divalent linking group, and

Z¹ represents a group having an acid group or a basic group.

In Formula (b1), the aromatic ring included in A¹ is the same as the above-mentioned aromatic ring preferably included in the pigment derivative B1. A¹ is preferably the group represented by Formula (A1) described above.

In Formula (b1), L¹ represents a single bond or a divalent linking group, and a divalent linking group is preferable. Examples of the divalent linking group represented by L¹ include an alkylene group, an arylene group, a heterocyclic group, —O—, —N(R^{L1})—, —NHCO—, —CONH—, —OCO—, —COO—, —CO—, —SO₂NH—, —SO₂—, and a group formed by a combination of these groups. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{L1} represents a hydrogen atom, an alkyl group, an alkenyl group, an alkynyl group, or an aryl group, and a hydrogen atom or an alkyl group is preferable and a hydrogen atom is more preferable. The preferred ranges of the alkyl group, alkenyl group, alkynyl group, and aryl group represented by R^{L1} are the same as the ranges described as the preferred ranges of the alkyl group, alkenyl group, alkynyl group, and aryl group of Ra¹.

The divalent linking group represented by L¹ is preferably a group represented by Formula (L1).

-L^{1A}-L^{1B}-L^{1C}- (L1)

In the formula, L^{1A} and L^{1B} each independently represent —O—, —N(R^{L1})—, —NHCO—, —CONH—, —OCO—, —COO—, —CO—, —SO₂NH—, or —SO₂—, and L^{1B} represents a single bond or a divalent linking group.

Examples of the divalent linking group represented by L^{1B} include an alkylene group, an arylene group, a group in which an alkylene group and an arylene group are bonded to each other through a single bond, —O—, —N(R^{L1})—, —NHCO—, —CONH—, —OCO—, —COO—, —CO—, —SO₂NH—, —SO₂—, or a group formed by a combination of these groups, and a group in which alkylene groups or arylene groups are bonded to each other through —O—, —N(R^{L1})—, —NHCO—, —CONH—, —OCO—, —COO—, —CO—, —SO₂NH—, —SO₂—, or a group formed by a combination of these groups.

Specific examples of L¹ include groups having the following structures.

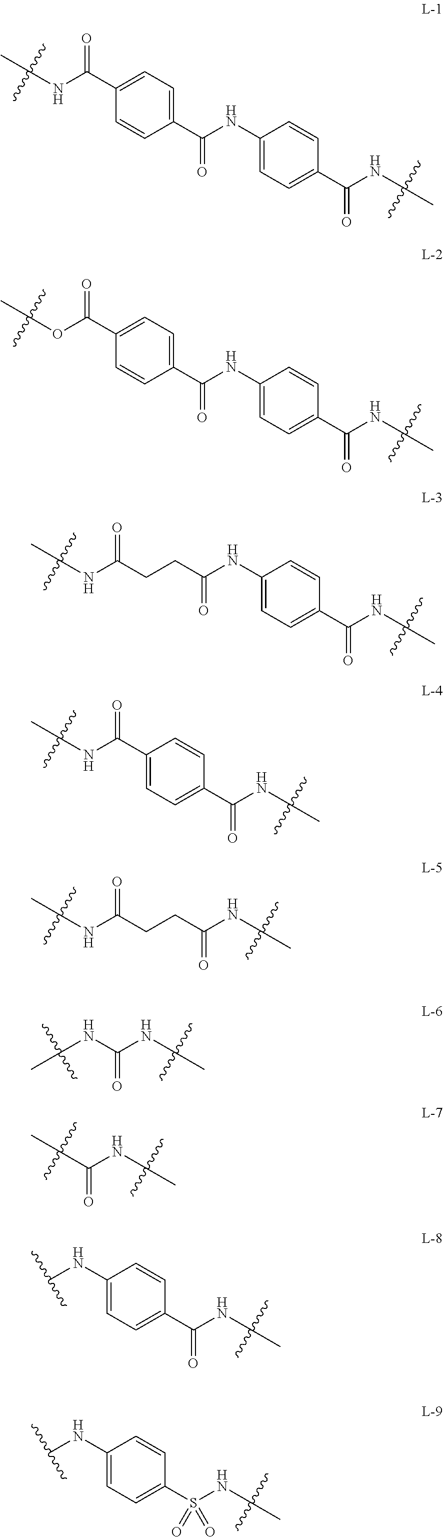

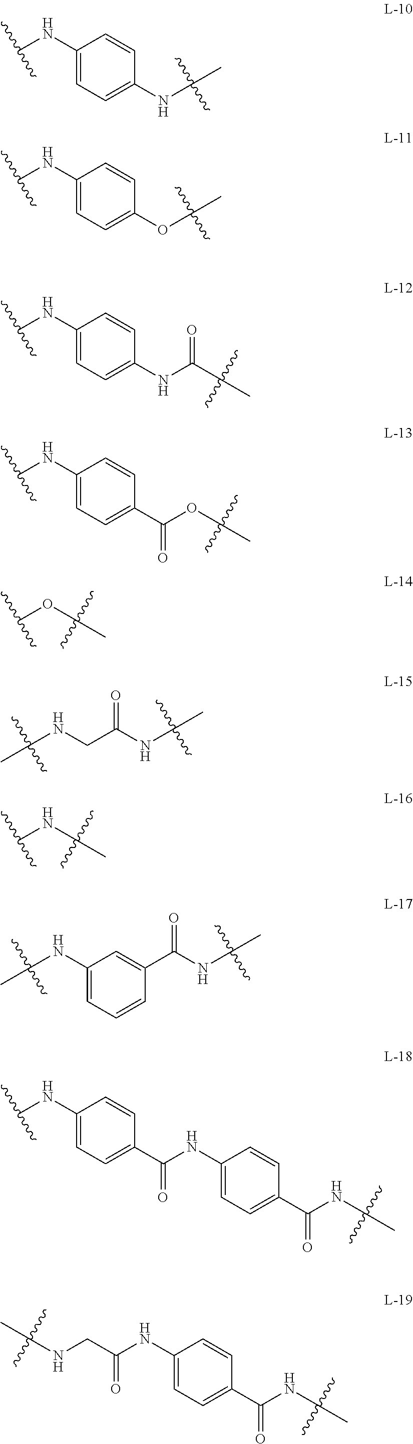

Z¹ in Formula (b1) represents a group having an acid group or a basic group. Examples of the types of the acid group and the basic group include the above-described groups.

Z¹ in Formula (b1) is preferably a group represented by Formula (Z1) or a group represented by Formula (Z10).

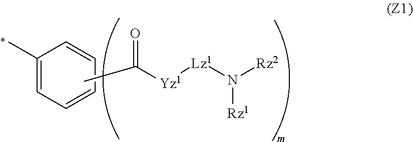

In Formula (Z1), * represents a bonding site.

Yz¹ represents —N(Ry¹)- or —O—, in which Ry¹ represents a hydrogen atom, an alkyl group, an alkenyl group, an alkynyl group, or an aryl group,

Lz¹ represents a divalent linking group,

Rz¹ and Rz² each independently represent a hydrogen atom, an alkyl group, an alkenyl group, an alkynyl group, or an aryl group, in which Rz¹ and Rz² may be bonded to each other through a divalent group to form a ring, and

m represents an integer of 1 to 5.

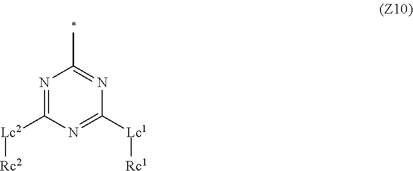

In Formula (Z10), * represents a bonding site. Lc¹ and Lc² each independently represent a single bond or a linking group, Rc¹ and Rc² each independently represent a substituent, and at least one of Rc¹ or Rc² represents an acid group or a basic group.

First, Formula (Z1) will be described.

In Formula (Z1), Yz¹ represents —N(Ry¹)- or —O—, and —N(Ry¹)- is preferable. Ry¹ represents a hydrogen atom, an alkyl group, an alkenyl group, an alkynyl group, or an aryl group, and a hydrogen atom or an alkyl group is preferable and a hydrogen atom is more preferable. The preferred ranges of the alkyl group, alkenyl group, alkynyl group, and aryl group represented by Ry¹ are the same as the ranges described as the preferred ranges of the alkyl group, alkenyl group, alkynyl group, and aryl group of Ra¹.

In Formula (Z1), examples of the divalent linking group represented by Lz¹ include an alkylene group, an arylene group, a heterocyclic group, —O—, —N(R^{L1})—, —NHCO—, —CONH—, —OCO—, —COO—, —CO—, —SO₂NH—, —SO₂—, and a group formed by a combination of these groups, and an alkylene group is preferable. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear.

In Formula (Z1), Rz¹ and Rz² each independently represent a hydrogen atom, an alkyl group, an alkenyl group, an alkynyl group, or an aryl group, and an alkyl group or an aryl group is preferable and an alkyl group is more preferable. The alkyl group preferably has 1 to 10 carbon atoms, more preferably has 1 to 5 carbon atoms, still more preferably has 1 to 3 carbon atoms, and particularly preferably has 1 or 2 carbon atoms. The alkyl group may be any of linear, branched, and cyclic forms, and is preferably linear or branched and more preferably linear. The alkenyl group preferably has 2 to 10 carbon atoms, more preferably has 2 to 8 carbon atoms, and particularly preferably has 2 to S carbon atoms. The alkenyl group may be any of linear, branched, and cyclic forms, and is preferably linear or branched and more preferably linear. The alkynyl group preferably has 2 to 10 carbon atoms, more preferably has 2 to 8 carbon atoms, and particularly preferably has 2 to S carbon atoms. The alkynyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms.

In Formula (Z1), Rz¹ and Rz² may be bonded to each other through a divalent group to form a ring. Examples of the divalent group include —CH₂—, —O—, and —SO₂—. Specific examples of the ring formed by bonding Rz¹ and Rz² to each other through the divalent group include the following.

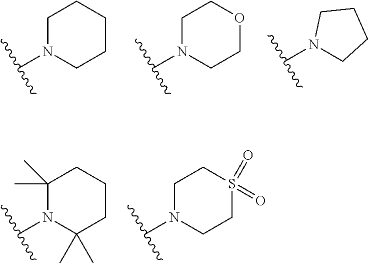

In Formula (Z1), m represents an integer of 1 to 5, and is preferably 1 to 4, more preferably 1 to 3, still more preferably 2 or 3, and particularly preferably 2.

The group represented by Formula (Z1) is preferably a group represented by Formula (Z2).

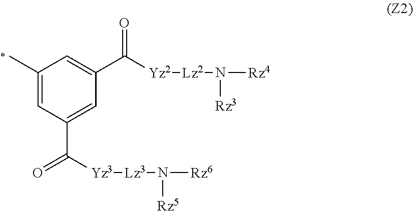

In Formula (Z2), * represents a bonding site,

Yz² and Yz³ each independently represent —N(Ry²)- or —O—, in which Ry² represents a hydrogen atom, an alkyl group, an alkenyl group, an alkynyl group, or an aryl group,

Lz² and Lz³ each independently represent a divalent linking group, and

Rz³ to Rz⁶ each independently represent a hydrogen atom, an alkyl group, an alkenyl group, an alkynyl group, or an aryl group, and

Rz³ and Rz⁴, and Rz⁵ and Rz⁶ may be respectively bonded to each other through a divalent group to form a ring.

Yz² and Yz³ in Formula (Z2) have the same meanings as Yz¹ in Formula (Z1), and the preferred ranges are also the same. Ry² represents a hydrogen atom, an alkyl group, an alkenyl group, an alkynyl group, or an aryl group, and a hydrogen atom or an alkyl group is preferable and a hydrogen atom is more preferable. The preferred ranges of the alkyl group, alkenyl group, alkynyl group, and aryl group represented by Ry² are the same as the ranges described as the preferred ranges of the alkyl group, alkenyl group, alkynyl group, and aryl group of Ra¹.

Lz² and Lz³ in Formula (Z2) have the same meanings as Lz¹ in Formula (Z1), and the preferred ranges are also the same. Rz³ to Rz⁶ in Formula (Z2) have the same meanings as Rz¹ and Rz² in Formula (Z1), and the preferred ranges are also the same.

Next, Formula (Z10) will be described.

In Formula (Z10), Lc¹ and Lc² each independently represent a single bond or a linking group, and a divalent linking group is preferable. Examples of the divalent linking group include an alkylene group, an arylene group, —O—, —N(R^{L1})—, —NHCO—, —CONH—, —OCO—, —COO—, —CO—, —SO₂NH—, —SO₂—, and a group formed by a combination of these groups. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{L1} represents a hydrogen atom, an alkyl group, or an aryl group, and a hydrogen atom or an alkyl group is preferable and a hydrogen atom is more preferable. The alkyl group represented by R^{L1} preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The alkyl group represented by R^{L1} may further have a substituent. Examples of the substituent include the above-described substituent T. The aryl group represented by R^{L1} preferably has 6 to 30 carbon atoms, more preferably has 6 to 20 carbon atoms, and still more preferably has 6 to 12 carbon atoms. The aryl group represented by R^{L1} may further have a substituent. Examples of the substituent include the above-described substituent T.

In Formula (Z10), Rc¹ and Rc² each independently represent a substituent. Examples of the substituent include an alkyl group, an aryl group, a heterocyclic group, a hydroxyl group, an acid group, and a basic group. However, at least one of Rc¹ or Rc² represents an acid group or a basic group. It is preferable that at least one of Rc¹ or Rc² is a basic group, and it is more preferable that both Rc¹ and Rc² are basic groups. Examples of the acid group and the basic group include those described above. The alkyl group preferably has 1 to 30 carbon atoms, more preferably has 1 to 15 carbon atoms, and still more preferably 1 to 8 carbon atoms. The alkyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms. The heterocyclic group may be a single ring or a fused ring. The heterocyclic group is preferably a single ring or a fused ring having 2 to 4 fused numbers. The number of heteroatoms constituting a ring of the heterocyclic group is preferably 1 to 3. The heteroatom constituting the ring of the heterocyclic group is preferably a nitrogen atom, an oxygen atom, or a sulfur atom. The number of carbon atoms constituting the ring of the heterocyclic group is preferably 3 to 30, more preferably 3 to 18, and more preferably 3 to 12. The alkyl group, aryl group, and heterocyclic group may further have a substituent. Examples of the substituent include the above-described substituent T.

The group represented by Formula (Z10) is preferably a group represented by Formula (Z11), and more preferably a group represented by Formula (Z12).

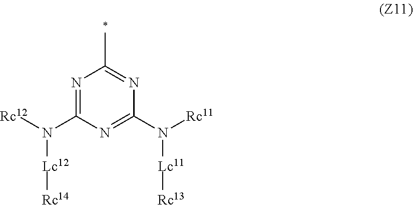

In Formula (Z11), * represents a bonding site, Lc¹¹ and Lc¹² each independently represent a single bond or a linking group, Rc¹¹ and Rc¹² each independently represent a hydrogen atom or a substituent, Rc¹³ and Rc¹⁴ each independently represent a substituent, and at least one of Rc¹³ or Rc¹⁴ represents an acid group or a basic group.

Rc¹³ and Rc¹⁴ in Formula (Z11) have the same meaning as Rc¹ and Rc² in Formula (Z10), and the preferred ranges are also the same.

In Formula (Z11), Rc¹¹ and Rc¹² each independently represent a hydrogen atom or a substituent. Examples of the substituent represented by Rc¹¹ and Rc¹² include an alkyl group and an aryl group. The alkyl group preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms. The alkyl group and aryl group may further have a substituent. Examples of the substituent include the above-described substituent T. Rc¹¹ and Rc¹² are preferably hydrogen atoms.

In Formula (Z11), Lc¹¹ and Lc¹² each independently represent a single bond or a linking group, and a divalent linking group is preferable. Examples of the divalent linking group include an alkylene group, an arylene group, —O—, —N(R^{L11})—, —NHCO—, —CONH—, —OCO—, —COO—, —CO—, —SO₂NH—, —SO₂—, and a group formed by a combination of these groups, and a group including at least one selected from an alkylene group or an arylene group is preferable, a group including an alkylene group is more preferable, and an alkylene group is still more preferable. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{L1} represents a hydrogen atom, an alkyl group, or an aryl group, and a hydrogen atom or an alkyl group is preferable and a hydrogen atom is more preferable. The alkyl group and aryl group represented by R^{L11} have the same meaning as the alkyl group and aryl group represented by R^{L1} described above.

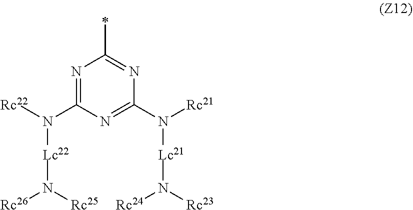

In Formula (Z12), * represents a bonding site, Lc²¹ and Lc²² each independently represent a single bond or a linking group. Rc²¹ and Rc²² each independently represent a hydrogen atom or a substituent, Rc²³ to Rc²⁶ each independently represent a hydrogen atom or a substituent, Rc²³ and Rc²⁴ may be bonded to each other through a divalent group to form a ring, and Rc²⁵ and Rc²⁶ may be bonded to each other through a divalent group to form a ring.

Rc²¹ and Rc²² in Formula (Z12) have the same meaning as Rc¹¹ and Rc¹² in Formula (Z11), and the preferred ranges are also the same. Lc²¹ and Lc²² in Formula (Z12) have the same meaning as Lc¹¹ and Lc¹² in Formula (Z11), and the preferred ranges are also the same.

In Formula (Z12), Rc²³ to Rc²⁶ each independently represent a hydrogen atom or a substituent, and a substituent is preferable. Examples of the substituent include an alkyl group and an aryl group, and an alkyl group is preferable. The alkyl group preferably has 1 to 10 carbon atoms, more preferably has 1 to 5 carbon atoms, and still more preferably has 1 to 3 carbon atoms. The alkyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms. The alkyl group and aryl group may further have a substituent. Examples of the substituent include the above-described substituent T.

In Formula (Z12), Rc²³ and Rc²⁴ may be bonded to each other through a divalent group to form a ring, and Rc²⁵ and Rc²⁶ may be bonded to each other through a divalent group to form a ring. Examples of the divalent group include —CH₂—, —O—, and —SO₂—. Specific examples of the ring formed by bonding the above-described groups to each other through the divalent group include the following.

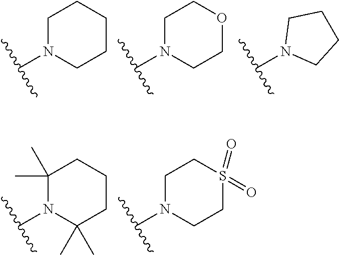

Specific examples of Z¹ include groups having the following structures. In the following structural formulae. Ph represents a phenyl group.

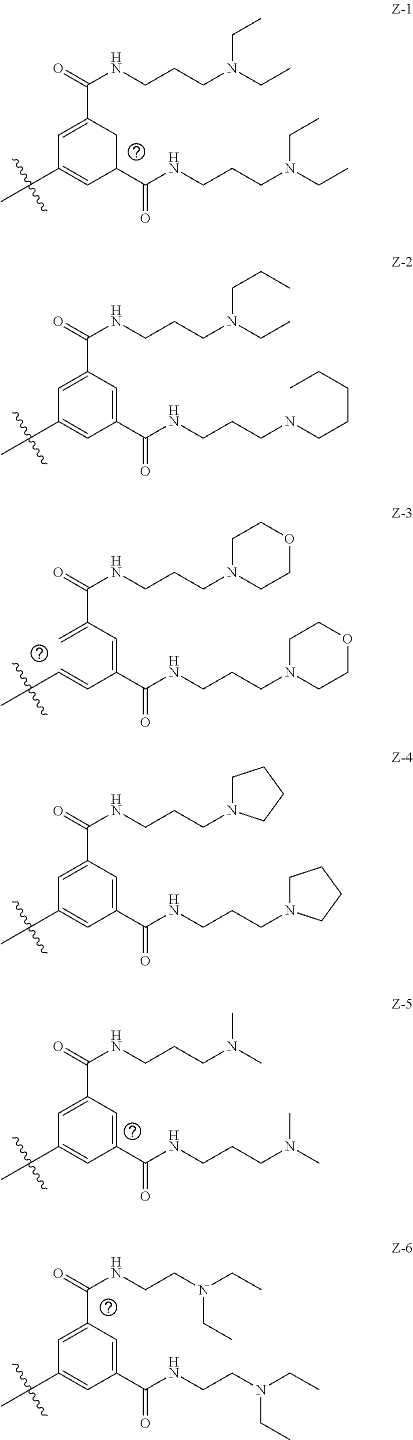 [SMILES: *C1=CC(C(=O)NCCCN(CC)CC)CC(C(=O)NCCCN(CC)CC)=C1 ; *c1cc(C(=O)NCCCNCCCCC)cc(C(=O)NCCCN(CC)CCC)c1 ; */C=C/C(=C\C(=C)C(=O)NCCCN1CCOCC1)C(=O)NCCCN1CCOCC1 ; *c1cc(C(=O)NCCCN2CCCC2)cc(C(=O)NCCCN2CCCC2)c1 ; *c1cc(C(=O)NCCCN(C)C)cc(C(=O)NCCCN(C)C)c1 ; *c1cc(C(=O)NCCN(CC)CC)cc(C(=O)NCCN(CC)CC)c1]

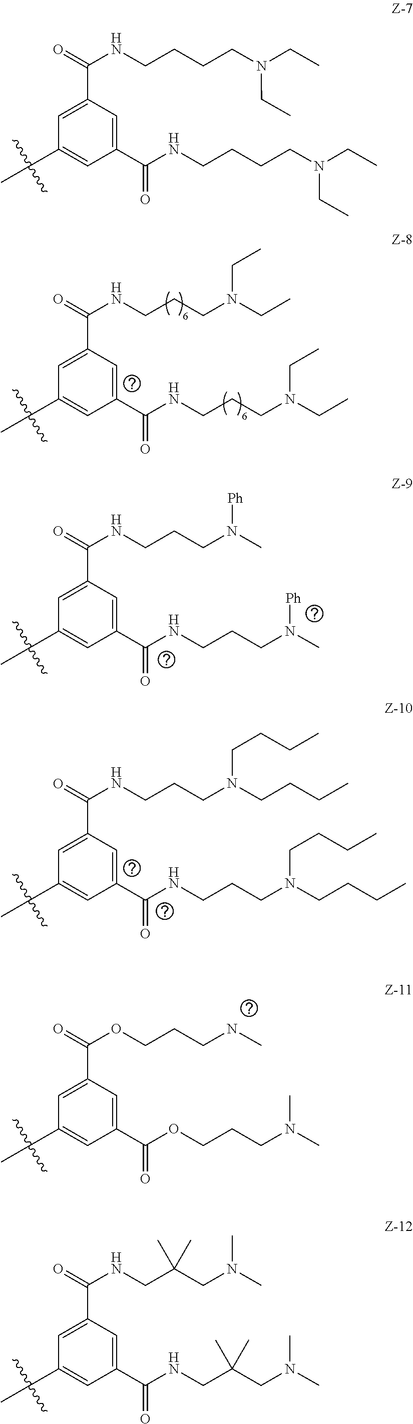

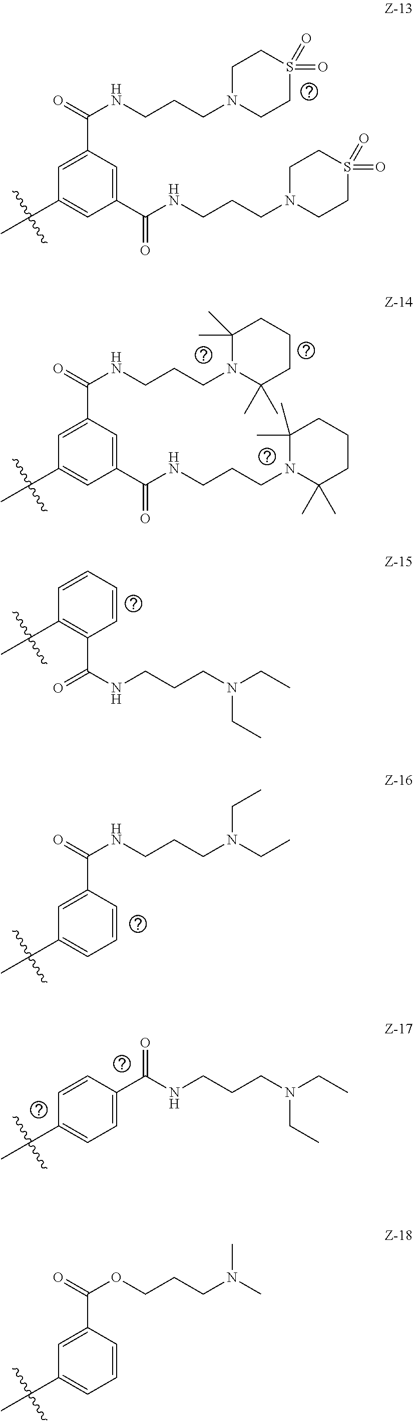 [SMILES: *c1cc(C(=O)NCCCN2CCS(=O)(=O)CC2)cc(C(=O)NCCCN2CCS(=O)(=O)CC2)c1 ; *c1cc(C(=O)NCCCN2C(C)(C)CCCC2(C)C)cc(C(=O)NCCCN2C(C)(C)CCCC2(C)C)c1 ; *c1ccccc1C(=O)NCCCN(CC)CC ; *c1cccc(C(=O)NCCCN(CC)CC)c1 ; *c1ccc(C(=O)NCCCN(CC)CC)cc1 ; *c1cccc(C(=O)OCCCN(C)C)c1]

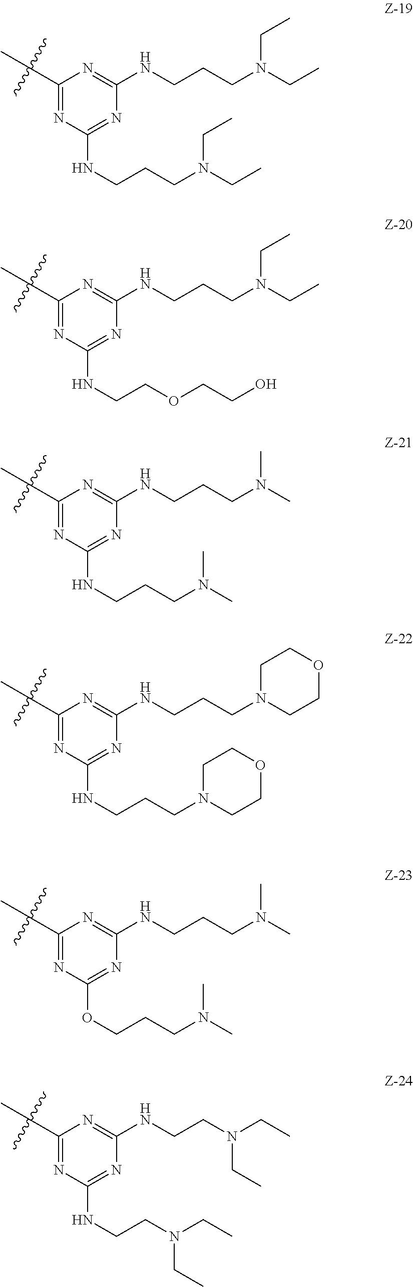

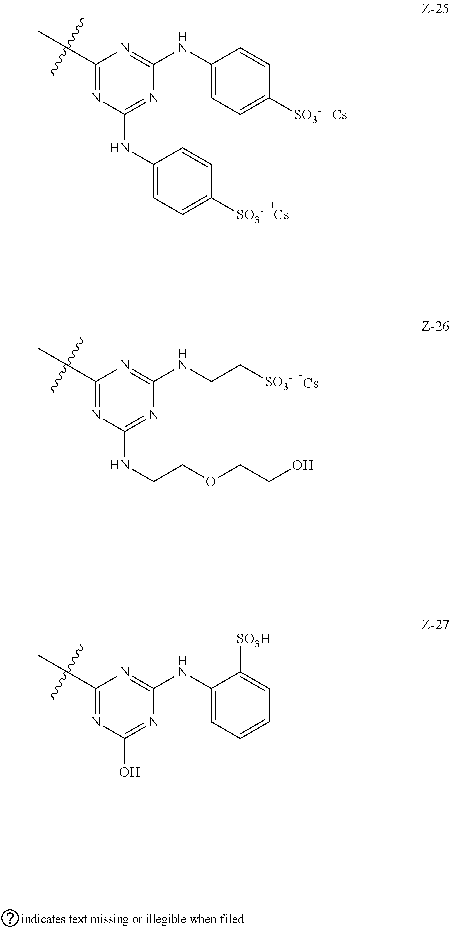

In the present invention, the pigment derivative B1 is preferably a compound represented by Formula (b2). By using such a compound, the effects of the present invention are more remarkably obtained.

A¹-X¹-L²-X²—Z¹ (b2)

In Formula (b2), A¹ represents a group including an aromatic ring,

X¹ and X² each independently represent a single bond, —O—, —N(R¹)—, —NHCO—, —CONH—, —OCO—, —COO—, —CO—, —SO₂NH—, or —SO₂—, where R¹ represents a hydrogen atom, an alkyl group, an alkenyl group, an alkynyl group, or an aryl group,

L² represents a single bond or a divalent linking group, and

Z¹ represents the group represented by Formula (Z1).

A¹ and Z¹ in Formula (b2) have the same meanings as A¹ and Z¹ in Formula (b1), and the preferred ranges are also the same.

X¹ and X² in Formula (b2) each independently represent a single bond, —O—, —N(R¹)—, —NHCO—, —CONH—, —OCO—, —COO—, —CO—, —SO₂NH—, or —SO₂—, —O—, —N(R¹)—, —NHCO—, —CONH—, —OCO—, —COO—, —CO—, —SO₂NH—, or —SO₂— is preferable. R¹ represents a hydrogen atom, an alkyl group, an alkenyl group, an alkynyl group, or an aryl group, and a hydrogen atom or an alkyl group is preferable and a hydrogen atom is more preferable. The preferred ranges of the alkyl group, alkenyl group, alkynyl group, and aryl group represented by R¹ are the same as the ranges described as the preferred ranges of the alkyl group, alkenyl group, alkynyl group, and aryl group of Ra¹.

L² in Formula (b2) represents a single bond or a divalent linking group. Examples of the divalent linking group represented by L² include an alkylene group, an arylene group, a group in which an alkylene group and an arylene group are bonded to each other through a single bond, —O—, —N(R²)—, —NHCO—, —CONH—, —OCO—, —COO—, —CO—, —SO₂NH—, —SO₂—, or a group formed by a combination of these groups, and a group in which alkylene groups or arylene groups are bonded to each other through —O—, —N(R²)—, —NHCO—, —CONH—, —OCO—, —COO—, —CO—, —SO₂NH—, —SO₂—, or a group formed by a combination of these groups. R² represents a hydrogen atom, an alkyl group, an alkenyl group, an alkynyl group, or an aryl group, and a hydrogen atom or an alkyl group is preferable and a hydrogen atom is more preferable. The preferred ranges of the alkyl group, alkenyl group, alkynyl group, and aryl group represented by R² are the same as the ranges described as the preferred ranges of the alkyl group, alkenyl group, alkynyl group, and aryl group of Ra¹.

Specific examples of the pigment derivative B1 include compounds shown below and compounds described in Examples described later. In the following table, the symbols described in the columns of structure of A¹, structure of L¹, and structure of Z¹ are the structures exemplified in the specific examples of A¹ (that is, specific examples of the group represented by Formula (A1)), the specific examples of L¹, and the specific examples of Z¹ respectively.

TABLE 1
A¹-L¹-Z¹

| Compound | Structure | Structure | Structure |
|---|---|---|---|
| No. | of A¹ | of L¹ | of Z¹ |
| C-1 | A-1 | L-1 | Z-1 |
| C-2 | A-2 | L-1 | Z-1 |
| C-3 | A-3 | L-1 | Z-1 |
| C-4 | A-4 | L-1 | Z-1 |
| C-5 | A-5 | L-1 | Z-1 |
| C-6 | A-6 | L-1 | Z-1 |
| C-7 | A-7 | L-1 | Z-1 |
| C-8 | A-8 | L-1 | Z-1 |
| C-9 | A-9 | L-1 | Z-1 |
| C-10 | A-10 | L-1 | Z-1 |
| C-11 | A-11 | L-1 | Z-1 |
| C-12 | A-12 | L-1 | Z-1 |
| C-13 | A-I3 | L-1 | Z-1 |
| C-I4 | A-14 | L-1 | Z-1 |
| C-15 | A-15 | L-1 | Z-1 |
| C-16 | A-16 | L-1 | Z-1 |
| C-17 | A-17 | L-1 | Z-1 |
| C-18 | A-18 | L-1 | Z-1 |
| C-19 | A-19 | L-1 | Z-1 |
| C-20 | A-20 | L-1 | Z-1 |
| C-21 | A-21 | L-1 | Z-1 |
| C-22 | A-22 | L-1 | Z-1 |
| C-23 | A.23 | L-1 | Z-1 |
| C-24 | A.24 | L-1 | Z-1 |
| C-25 | A125 | L-2 | Z-1 |
| C-26 | A-26 | L-2 | Z-1 |
| C-27 | A-16 | L-3 | Z-1 |
| C-28 | A-16 | L-4 | Z-1 |
| C-29 | A-16 | L-5 | Z-1 |
| C-30 | A-16 | L-6 | Z-1 |
| C-31 | A-27 | L-7 | Z-1 |
| C-32 | A-28 | L-7 | Z-1 |
| C-33 | A-29 | L-8 | Z-1 |
| C-34 | A-30 | L-8 | Z-1 |
| C-35 | A-31 | L-8 | Z-1 |
| C-36 | A-32 | L-8 | Z-1 |
| C-37 | A-33 | L-8 | Z-1 |
| C-38 | A-34 | L-8 | Z-1 |
| C-39 | A-35 | L-8 | Z-1 |
| C-40 | A-36 | L-8 | Z-1 |
| C-41 | A-37 | L-8 | Z-1 |
| C-47 | A-38 | L-8 | Z-1 |
| C-43 | A-39 | L-8 | Z-1 |
| C-44 | A-40 | L-8 | Z-1 |
| C-45 | A-41 | L-8 | Z-1 |
| C-46 | A-42 | L-8 | Z-1 |
| C-47 | A-43 | L-8 | Z-1 |
| C-48 | A-44 | L-8 | Z-1 |
| C-49 | A-45 | L-8 | Z-1 |
| C-50 | A-46 | L-8 | Z-1 |
| C-51 | A-47 | L-8 | Z-1 |
| C-52 | A-48 | L-8 | Z-1 |
| C-53 | A-49 | L-8 | Z-1 |
| C-54 | A-50 | L-8 | Z-1 |
| C-55 | A-51 | L-8 | Z-1 |
| C-56 | A-52 | L-8 | Z-1 |
| C-57 | A-53 | L-8 | Z-1 |
| C-58 | A-54 | L-8 | Z-1 |
| C-59 | A-55 | L-8 | Z-1 |
| C-60 | A-56 | L-8 | Z-1 |
| C-61 | A-57 | L-8 | Z-1 |
| C-67 | A-58 | L-8 | Z-1 |
| C-63 | A-32 | L-9 | Z-1 |
| C-64 | A-32 | L-10 | Z-1 |
| C-65 | A-32 | L-11 | Z-1 |
| C-66 | A-32 | L-12 | Z-1 |
| C-67 | A-32 | L-13 | Z-1 |
| C-68 | A-32 | L-14 | Z-1 |
| C-69 | A-32 | L-15 | Z-1 |
| C-70 | A-32 | L-16 | Z-1 |
| C-71 | A-37 | L-17 | Z-1 |
| C-72 | A-32 | L-18 | Z-1 |
| C-73 | A-32 | L-19 | Z-1 |
| C-74 | A-32 | L-8 | Z-2 |
| C-75 | A-32 | L-8 | Z-3 |
| C-76 | A-32 | L-8 | Z-4 |
| C-77 | A-32 | L-8 | Z-5 |
| C-78 | A-32 | L-8 | Z-6 |
| C-79 | A-32 | L-8 | Z-7 |
| C-80 | A-32 | L-8 | Z-8 |
| C-81 | A-31 | L-8 | Z-9 |
| C-82 | A-32 | L-8 | Z-10 |
| C-83 | A-32 | L-8 | Z-11 |
| C-84 | A-32 | L-8 | Z-12 |
| C-85 | A-32 | L-8 | Z-13 |
| C-86 | A-32 | L-8 | Z-14 |
| C-87 | A-37 | L-8 | Z-15 |
| C-88 | A-32 | L-8 | Z-16 |
| C-89 | A-32 | L-8 | Z-17 |
| C-90 | A-32 | L-8 | Z-18 |
| C-91 | A-16 | L-8 | Z-17 |
| C-92 | A-15 | L-8 | Z-17 |
| C-93 | A-59 | L-8 | Z-1 |
| C-94 | A-60 | L-8 | Z-1 |
| C-95 | A-61 | L-8 | Z-1 |
| C-96 | A-62 | L-8 | Z-1 |
| C-97 | A-63 | L-8 | Z-1 |
| C-98 | A-64 | L-8 | Z-1 |
| C-99 | A-65 | L-8 | Z-1 |

It is also preferable that the pigment derivative B used in the photosensitive composition according to the embodiment of the present invention further includes a pigment derivative (hereinafter, also referred to as a pigment derivative B2) in which the maximum value of a molar absorption coefficient in a wavelength range of 400 to 700 nm is more than 3000 L·mol⁻¹·cm⁻¹. By using the pigment derivative B1 and the pigment derivative B2 in combination, color density of the film can be increased. Therefore, it is particularly effective in a case where the photosensitive composition according to the embodiment of the present invention is used as a composition for forming a colored pixel of a color filter. In a case where the pigment derivative B1 and the pigment derivative B2 are used in combination, a content of the pigment derivative B2 is preferably 10 to 100 parts by mass, more preferably 15 to 90 parts by mass, and still more preferably 20 to 80 parts by mass with respect to 100 parts by mass of the pigment derivative 81.

Examples of the pigment derivative B2 include a compound having a structure in which a part of a chromophore is substituted with an acid group or a basic group. Examples of the chromophore constituting the pigment derivative B2 include a quinoline skeleton, a benzimidazolone skeleton, a diketopyrrolopyrrole skeleton, an azo skeleton, a phthalocyanine skeleton, an anthraquinone skeleton, a quinacridone skeleton, a dioxazine skeleton, a perinone skeleton, a perylene skeleton, a thioindigo skeleton, an isoindoline skeleton, an isoindolinone skeleton, a quinophthalone skeleton, a threne skeleton, and a metal complex skeleton. Among these, a quinoline skeleton, a benzimidazolone skeleton, a diketopyrrolopyrrole skeleton, an azo skeleton, a quinophthalone skeleton, an isoindoline skeleton, or a phthalocyanine skeleton is preferable, and an azo skeleton or a benzimidazolone skeleton is more preferable. Specific examples of the pigment derivative B2 include pigment derivatives described in Example described later and compounds described in JP1981-118462A (JP-S56-118462A), JP1988-264674A (JP-S63-264674A), JP1989-217077A (JP-H01-217077A), JP1991-009961A (JP-H03-009% 1A), JP1991-026767A (JP-H03-026767A), JP1991-153780A (JP-H03-153780A), JP1991-045662A (JP-H03-045662A), JP1992-285669A (JP-H04-285669A), JP1994-145546A (JP-H06-145546A), JP1994-212088A (JP-H06-212088A), JP1994-240158A (JP-H06-240158A), JP1998-030063A (JP-H10-030063A), JP1998-195326A (JP-H10-195326A), paragraph Nos. 0086 to 0098 of WO2011/024896A, paragraph Nos. 0063 to 0094 of WO2012/102399A, paragraph No. 0082 of WO2017/038252A, paragraph No. 0171 of JP2015-151530A, paragraph Nos. 0162 to 0183 of JP2011-252065A, JP2003-081972A, JP5299151B, JP2015-172732A, JP2014-199308A, JP2014-085562A, JP2014-035351A, JP2008-081565A, JP2019-109512A, and JP2019-133154A.

The total content of the coloring material A and the pigment derivative B in the total solid content of the photosensitive composition is 50% by mass or more. The effects of the present invention are more remarkable in a case where the total content of the coloring material A and the pigment derivative B is high. The total content of the coloring material A and the pigment derivative B is preferably 55% by mass or more, more preferably 60% by mass or more, still more preferably 65% by mass or more, and particularly preferably 70% by mass or more. In addition, the upper limit of the total content of the coloring material A and the pigment derivative B is preferably 85% by mass or less, more preferably 82.5% by mass or less, and still more preferably 80% by mass or less.

The content of the pigment derivative B in the total solid content of the photosensitive composition is preferably 0.3% to 20% by mass. The lower limit is preferably 0.6% by mass or more and more preferably 0.9% by mass or more. The upper limit is preferably 15% by mass or less, more preferably 12.5% by mass or less, and still more preferably 10% by mass or less.

In addition, the content of the pigment derivative B is preferably 3 to 30 parts by mass with respect to 100 parts by mass of the total of the coloring material A and the pigment derivative B. The lower limit is preferably 4 parts by mass or more and more preferably 5 parts by mass or more. The upper limit is preferably 25 parts by mass or less and more preferably 20 parts by mass or less.

In addition, the content of the above-described pigment derivative B1 in the total solid content of the photosensitive composition is preferably 0.3% to 20% by mass. The lower limit is preferably 0.6% by mass or more and more preferably 0.9% by mass or more. The upper limit is preferably 15% by mass or less, more preferably 12.5% by mass or less, and still more preferably 10% by mass or less.

<<Dispersant C>>

The photosensitive composition according to the embodiment of the present invention contains a dispersant C. The dispersant C includes a dispersant C1 (hereinafter, also referred to as a dispersant C1) having an ethylenically unsaturated bond-containing group. Examples of the ethylenically unsaturated bond-containing group included in the dispersant C1 include a vinyl group, a (meth)allyl group, a (meth)acryloyl group, and a vinylphenyl group. Among these, from the viewpoint of reactivity, a (meth)acryloyl group or a vinylphenyl group is preferable, and a (meth)acryloyl group is more preferable.

A weight-average molecular weight (Mw) of the dispersant C1 is preferably 3000 to 100000, more preferably 5000 to 50000, and still more preferably 7000 to 30000.

From the viewpoint of developability and curing properties, an ethylenically unsaturated bond-containing group value (hereinafter, also referred to as C═C value) of the dispersant C1 is preferably 0.01 to 2.0 mmol/g, more preferably 0.1 to 1.5 mmol/g, and still more preferably 0.1 to 1.0 mmol/g. The C═C value of the dispersant C1 refers to a molar amount of ethylenically unsaturated bond-containing groups per 1 g of the solid content of the dispersant C1.

From the viewpoint of developability and dispersibility of the pigment, an acid value of the dispersant C1 is preferably 20 to 100 mgKOH/g, more preferably 30 to 90 mgKOH/g, and still more preferably 30 to 80 mgKOH/g.

(Specific Resin 1)

As the dispersant C1, it is preferable to use a resin (hereinafter, also referred to as a specific resin 1) which satisfies at least one of the following requirement 1 or the following requirement 2.

Requirement 1: the resin includes a constitutional unit having, in the same side chain, an anionic structure, a quaternary ammonium cationic structure which is ionically bonded to the anionic structure, and an ethylenically unsaturated bond-containing group.

Requirement 2: the resin includes a constitutional unit having, in a side chain, a quaternary ammonium cationic structure and a group to which an ethylenically unsaturated bond-containing group is linked.

[Requirement 1]

In the above-described requirement 1, with regard to the constitutional unit having, in the same side chain, an anionic structure, a quaternary ammonium cationic structure which is ionically bonded to the anionic structure, and an ethylenically unsaturated bond-containing group, the anionic structure and the quaternary ammonium cationic structure may be ionically bonded or dissociated.

In addition, the side chain in the requirement 1 may have at least one anionic structure, quaternary ammonium cationic structure, and ethylenically unsaturated bond-containing group, respectively, or may have a plurality of at least one selected from the group consisting of an anionic structure, a quaternary ammonium cationic structure, and an ethylenically unsaturated bond-containing group in one side chain.

—Anionic Structure—

The anionic structure in the above-described requirement 1 is not particularly limited, and examples thereof include anions derived from an acid group, such as carboxylate anion, sulfonate anion, phosphonate anion, phosphinate anion, and phenolate anion. Among these, carboxylate anion is preferable.

In addition, the anionic structure in the requirement 1 may be directly linked to the main chain of the specific resin 1. For example, in a case where a carboxyl group included in a constitutional unit derived from acrylic acid in an acrylic resin is anionized, the structure is an anionic structure directly linked to the main chain of the specific resin 1.

In addition, the distance (number of atoms) between the main chain and the quaternary ammonium cationic structure in a case where the anionic structure and the quaternary ammonium cationic structure are bonded to each other is preferably 4 to 70 elements, more preferably 4 to 50 elements, and still more preferably 4 to 30 elements. In the present specification, the distance between two structures in a polymer compound means the number of atoms of a linking group which links the two structures at the shortest.

The distance between the quaternary ammonium cationic structure and the ethylenically unsaturated bond-containing group is preferably 2 to 30 elements, more preferably 3 to 20 elements, and still more preferably 4 to 15 elements. The distance between the ethylenically unsaturated bond-containing group and the main chain is preferably 6 to 100 elements, more preferably 6 to 70 elements, and still more preferably 6 to 50 elements.

—Quaternary Ammonium Cationic Structure (Requirement 1)—

As the quaternary ammonium cationic structure in the above-described requirement 1, a structure in which at least three of four groups including four carbon atoms bonded to the nitrogen atom are hydrocarbon groups is preferable, and it is more preferable that at least three thereof are alkyl groups.

Among the above-described four groups including four carbon atoms bonded to the nitrogen atom, at least one thereof is a linking group including a bonding site with the ethylenically unsaturated bond-containing group. The above-described linking group is preferably a divalent to hexavalent linking group, more preferably a divalent to tetravalent linking group, and still more preferably a divalent or trivalent linking group. Examples of the above-described linking group include a group represented L^{A2} in Formula (A1) described later.

In addition, among the above-described four groups including four carbon atoms bonded to the nitrogen atom, it is preferable that only one thereof is the above-described linking group.

Among the above-described four groups including four carbon atoms, it is preferable that two or three thereof are alkyl groups having 1 to 4 carbon atoms, and it is preferable that two thereof are alkyl groups having 1 to 4 carbon atoms and one of the remaining two groups is a hydrocarbon group having 4 to 20 carbon atoms. In addition, the above-described two or three alkyl groups may be the same group or different groups.

As the above-described alkyl group having 1 to 4 carbon atoms, a methyl group or an ethyl group is preferable, and a methyl group is more preferable.

As the above-described hydrocarbon group having 4 to 20 carbon atoms, an alkyl group having 4 to 20 carbon atoms or a benzyl group is preferable.

In the above-described requirement 1, in a case where the side chain includes a plurality of quaternary ammonium cationic structures, the quaternary ammonium cationic structures may be bonded to each other through a linking group to form a ring structure. Examples of the ring structure formed include a ring structure represented by the following formulae. In the following formulae, * represents a bonding site with a linking group which includes a bonding site with the ethylenically unsaturated bond-containing group.

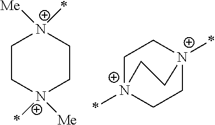 [SMILES: *[N+]1(C)CC[N+](*)(C)CC1 ; *[N+]12CC[N+](*)(CC1)CC2]

[Requirement 2]

In the side chain in the above-described requirement 2, the quaternary ammonium cationic structure and the ethylenically unsaturated bond-containing group are linked to each other. That is, one side chain has both at least one quaternary ammonium cationic structure and at least one ethylenically unsaturated bond-containing group.

The side chain in the requirement 2 may have at least one quaternary ammonium cationic structure and ethylenically unsaturated bond-containing group, respectively, or may have a plurality of at least one selected from the group consisting of a quaternary ammonium cationic structure and an ethylenically unsaturated bond-containing group in one side chain.

In addition, the distance (number of atoms) between the main chain and the quaternary ammonium cationic structure is preferably 4 to 20 elements, more preferably 4 to 15 elements, and most preferably 4 to 10 elements.

The distance between the quaternary ammonium cationic structure and the polymerizable group is preferably 2 to 30 elements, more preferably 3 to 20 elements, and still more preferably 4 to 15 elements.

The distance between the polymerizable group and the main chain is preferably 6 to 50 elements, more preferably 6 to 30 elements, and still more preferably 6 to 20 elements.

—Quaternary Ammonium Cationic Structure (Requirement 2)—

As the quaternary ammonium cationic structure in the above-described requirement 2, a structure in which at least two of four groups including four carbon atoms bonded to the nitrogen atom are hydrocarbon groups is preferable, and it is more preferable that at least two thereof are alkyl groups.

As the above-described hydrocarbon group, an alkyl group or an aryl group is preferable, and an alkyl group or a phenyl group is more preferable.

As the above-described alkyl group, an alkyl group having 1 to 4 carbon atoms is preferable, a methyl group or an ethyl group is more preferable, and a methyl group is still more preferable. In addition, the above-described two alkyl groups may be the same group or different groups.

Among the above-described four groups including four carbon atoms bonded to the nitrogen atom, at least one thereof is a linking group including a bonding site with the ethylenically unsaturated bond-containing group, and at least one thereof is a linking group including a bonding site with the main chain in the specific resin 1.

The linking group with the ethylenically unsaturated bond-containing group is preferably a divalent to hexavalent linking group, more preferably a divalent to tetravalent linking group, and still more preferably a divalent or trivalent linking group. Examples of the above-described linking group include a group represented L^{B2} in Formula (B1) described later.

The linking group including a bonding site with the main chain in the specific resin 1 is preferably a divalent linking group. Examples of the above-described linking group include a group represented L^{B1} in Formula (B1) described later.

The counter anion of the quaternary ammonium cationic structure in the requirement 2 may be present in the specific resin 1, or in other components included in the curable composition, but it is preferable to be present in the specific resin 1.

[Constitutional Unit Represented by Formula (A1) and Constitutional Unit Represented by Formula (B1)]

It is preferable that the specific resin 1 includes at least one of a constitutional unit represented by Formula (A1) or a constitutional unit represented by Formula (B1).

A resin including the constitutional unit represented by Formula (A1) is a resin satisfying the requirement 1, and a resin including the constitutional unit represented by Formula (B1) is a resin satisfying the requirement 2.

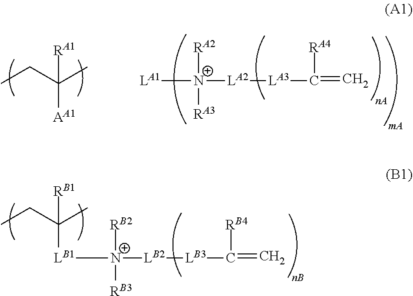

In Formula (A1), R^{A1} represents a hydrogen atom or an alkyl group,

A^{A1} represents a structure including a group in which a proton is dissociated from an acid group,

R^{A2} and R^{A3} each independently represent an alkyl group or an aralkyl group,

L^{A1} represents a monovalent substituent in a case where mA is 1, or represents an mA-valent linking group in a case where mA is 2 or more,

L^{A2} represents an (nA+1)-valent linking group,

L^{A3} represents a divalent linking group,

R^{A4} represents a hydrogen atom or an alkyl group,

nA represents an integer of 1 or more, and

mA represents an integer of 1 or more,

where in a case where mA is 2 or more, two or more R^{A2}'s, two or more R^{A3}'s, and two or more L^{A2}'s may be the same or different from each other,

in a case where mA is 2 or more, at least one of mA pieces of structures including a quaternary ammonium cation, which is selected from the group consisting of R^{A2} and R^{A3} included in one structure, may form a ring structure with at least one selected from the group consisting of R^{A2} and R^{A3} included in another structure,

in a case where at least one selected from the group consisting of nA and mA is 2 or more, two or more L^{A3}'s and two or more R^{A4}'s may be the same or different from each other, and

at least two of R^{A2}, R^{A3}, or L^{A2} may be bonded to each other to form a ring;

in Formula (B1), R^{B1} represents a hydrogen atom or an alkyl group,

L^{B1} represents a divalent linking group,

R^{B2} and R^{B3} each independently represent an alkyl group,

L^{B2} represents an (nB+1)-valent linking group,

L^{B3} represents a divalent linking group,

R^{B4} represents a hydrogen atom or an alkyl group, and

nB represents an integer of 1 or more,

where in a case where nB is 2 or more, two or more L^{B3}'s and two or more R^{B4}'s may be the same or different from each other, and

at least two of R^{B2}, R^{B3}, L^{B1}, or L^{B2} may be bonded to each other to form a ring.

In Formula (A1), R^{A1} is preferably a hydrogen atom or an alkyl group having 1 to 4 carbon atoms and more preferably a hydrogen atom or a methyl group.

In Formula (A1), A^{A1} represents a structure including a group in which a proton is dissociated from an acid group, and examples of the acid group include a carboxyl group, a sulfo group, a phosphoric acid group, a phosphonic acid group, and a phenolic hydroxyl group, and a carboxyl group is preferable. The number of acid groups included in A^{A1} may be one or plural, and it is preferable to be one. In addition, the acid group in A^{A1} may be bonded to a carbon atom to which R^{A1} in Formula (A1) is bonded directly or through a linking group. As the above-described linking group, a hydrocarbon group, an ether bond (—O—), an ester bond (—COO—), an amide bond (—CONH—), or a group in which two or more of these are bonded is preferable. Examples of the above-described hydrocarbon group include a divalent hydrocarbon group, and an alkylene group or an arylene group is preferable, and an alkylene group having 1 to 20 carbon atoms or a phenylene group is more preferable. In addition, in the present specification, unless otherwise specified, a hydrogen atom in the amide bond may be replaced with a known substituent such as an alkyl group and an aryl group.

In Formula (A1), R^{A2} and R^{A3} are each independently preferably an alkyl group, more preferably an alkyl group having 1 to 10 carbon atoms, still more preferably an alkyl group having 1 to 4 carbon atoms, particularly preferably a methyl group or an ethyl group, and most preferably a methyl group.

In Formula (A1), in a case where R^{A2} or R^{A3} is an aralkyl group, an aralkyl group having 7 to 22 carbon atoms is preferable, an aralkyl group having 7 to 10 carbon atoms is more preferable, and a benzyl group is still more preferable.

In Formula (A1), in a case where mA is 2 or more, L^{A1} is preferably an mA-valent hydrocarbon group, and more preferably a saturated aliphatic hydrocarbon, an aromatic hydrocarbon, or a group that mA hydrogen atoms are removed from or a structure in which two or more of these are bonded. In a case where mA is 1, L^{A1} is preferably an alkyl group, an aryl group, or an aralkyl group, and more preferably an alkyl group having 4 to 20 carbon atoms or a benzyl group.

In Formula (A1), L^{A2} is preferably any one of groups represented by Formulae (C1-1) to (C4-1) described later.

In Formula (A1), L^{A3} is preferably an ether bond (—O—), an ester bond (—COO—), an amide bond (—NHCO—), an alkylene group, or an arylene group, and more preferably an ester bond or a phenylene group.

In Formula (A1), R^{A4} is preferably a hydrogen atom or an alkyl group having 1 to 4 carbon atoms and more preferably a hydrogen atom or a methyl group.

In Formula (A1), nA is preferably 1 to 10, more preferably 1 to 4, still more preferably 1 or 2, and particularly preferably 1.

In Formula (A1), mA is preferably 1 to 10, more preferably 1 to 4, and still more preferably 1 to 3.

In Formula (B1), R^{B1} is preferably a hydrogen atom or an alkyl group having 1 to 4 carbon atoms and more preferably a hydrogen atom or a methyl group.

In Formula (B1), L^{B1} represents a divalent linking group, and a hydrocarbon group, an ether bond (—O—), an ester bond (—COO—), an amide bond (—CONH—), or a group in which two or more of these are bonded is preferable. Examples of the above-described hydrocarbon group include a divalent hydrocarbon group, and an alkylene group or an arylene group is preferable, and an alkylene group having 1 to 20 carbon atoms or a phenylene group is more preferable.

In Formula (B1), R^{B2} and R^{B3} are each independently preferably an alkyl group having 1 to 10 carbon atoms, more preferably an alkyl group having 1 to 4 carbon atoms, still more preferably a methyl group or an ethyl group, and particularly preferably a methyl group.

In Formula (B1), L^{B2} is preferably any one of groups represented by Formulae (C1-1) to (C4-1) described later.

In Formula (B1), L^{B3} is preferably an ether bond (—O—), an ester bond (—COO—), an amide bond (—NHCO—), an alkylene group, or an arylene group, and more preferably an ester bond or a phenylene group.

In Formula (B1), nB is preferably 1 to 10, more preferably 1 to 4, still more preferably 1 or 2, and particularly preferably 1.

L^{A2} in Formula (A1) or L^{B2} in Formula (B1) is preferably any one of groups represented by Formulae (C1-1) to (C4-1).

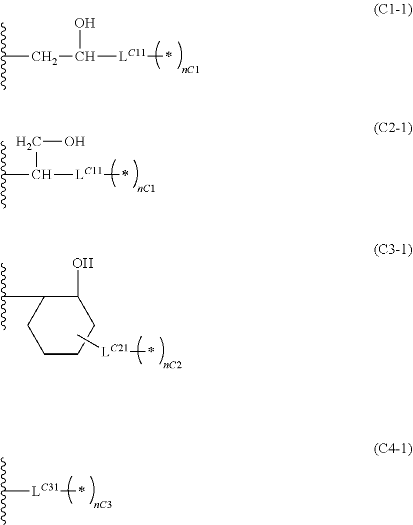

In Formulae (C1-1) to (C4-1), L^{C11} represents an (nC1+1)-valent linking group. L^{C21} represents an (nC2+1)-valent linking group, L^{C31} represents an (nC3+1)-valent hydrocarbon group, nC1 to nC3 each independently represent an integer of 1 or more, a wavy line part represents a bonding site with the nitrogen atom in Formula (A1) or Formula (B1), and * represents a bonding site with the carbon atom to which R^{A4} in Formula (A1) is bonded or the carbon atom to which R^{B4} in Formula (B1) is bonded.

In addition, in Formula (C3-1), it is sufficient that L^{C21} is bonded to any carbon atom of the cyclohexane ring in Formula (C3-1).

In Formula (C1-1) or Formula (C2-1), L^{C11} is preferably an (nC1+1)-valent hydrocarbon group, an ether bond, an ester bond, or a group in which two or more of these are bonded, and more preferably a saturated aliphatic hydrocarbon, an aromatic hydrocarbon, an ether bond, an ester bond, or a group that nC1+1 hydrogen atoms are removed from a structure in which two or more of these are bonded.

In Formula (C1-1) or Formula (C2-1), nC1 is preferably 1 to 10, more preferably 1 to 4, and still more preferably 1 or 2.

In Formula (C3-1), L^{C21} is preferably an (nC2+1)-valent hydrocarbon group, an ether bond, an ester bond, or a group in which two or more of these are bonded, and more preferably a saturated aliphatic hydrocarbon, an aromatic hydrocarbon, an ether bond, an ester bond, or a group that nC2+1 hydrogen atoms are removed from a structure in which two or more of these are bonded.

In Formula (C3-1), nC2 is preferably 1 to 10, more preferably 1 to 4, and still more preferably 1 or 2.

In Formula (C4-1), L^{C31} is preferably a saturated aliphatic hydrocarbon, an aromatic hydrocarbon, or a group that nC3+1 hydrogen atoms are removed from or a structure in which two or more of these are bonded.

In Formula (C4-1), nC3 is preferably 1 to 10, more preferably 1 to 4, and still more preferably 1 or 2.

In Formula (A1), in a case where L^{A2} is a group represented by Formula (C1-1). Formula (C2-1), or Formula (C3-1), L^{A2} is preferably an ester bond.

In Formula (A1), in a case where L^{A2} is a group represented by Formula (C4-1), L^{A3} is preferably a phenylene group.

In Formula (B1), in a case where L^{B2} is a group represented by Formula (C1-1). Formula (C2-1), or Formula (C3-1), L^{B2} is preferably an ester bond.

In Formula (B1), in a case where L^{B2} is a group represented by Formula (C4-1), L^{B3} is preferably a phenylene group.

In a case where the specific resin 1 includes at least one of the constitutional unit represented by Formula (A1) or the constitutional unit represented by Formula (B1), it is preferable that nA in Formula (A1) is 1 and a bond between L^{A2} and L^{A3} represents any one of groups represented by Formulae (C1) to (C4), or nB in Formula (B1) is 1 and L^{B2} and L^{B3} represent any one of groups represented by Formulae (C1) to (C4).

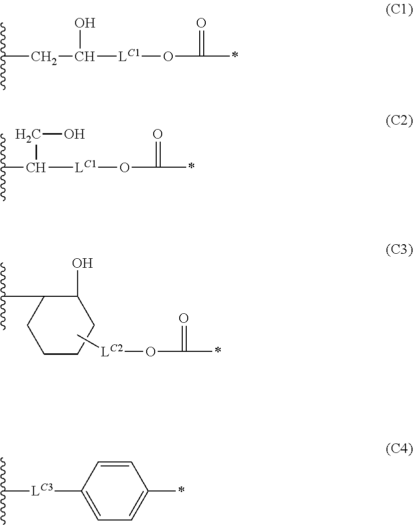

In Formulae (C1) to (C4), L^{C1}, L^{C2}, and L^{C3} each independently represent a single bond or a divalent linking group,

a wavy line part represents a bonding site with a nitrogen atom in Formula (A1) or Formula (B1), and

* represents a bonding site with a carbon atom to which R^{A4} in Formula (A1) is bonded or a carbon atom to which R^{B4} in Formula (B1) is bonded.

In addition, in Formula (C3), it is sufficient that L^{C2} is bonded to any carbon atom of the cyclohexane ring in Formula (C3).

In Formula (C1) or Formula (C2), L^{C1} is preferably a divalent hydrocarbon group, an ether bond, an ester bond, or a group in which two or more of these are bonded, more preferably an alkylene group, an arylene group, an ether bond, an ester bond, or a group in which two or more of these are bonded, and more preferably an alkylene group having 1 to 20 carbon atoms, a phenylene group, an ether bond, or a group in which two or more of these are bonded.

In Formula (C3), L^{C2} is preferably a divalent hydrocarbon group, an ether bond, an ester bond, or a group in which two or more of these are bonded, more preferably an alkylene group, an arylene group, an ether bond, an ester bond, or a group in which two or more of these are bonded, and more preferably an alkylene group having 1 to 20 carbon atoms, a phenylene group, an ether bond, or a group in which two or more of these are bonded.

In Formula (C4), L^{C3} is preferably a divalent hydrocarbon group, an ether bond, an ester bond, or a group in which two or more of these are bonded, more preferably an alkylene group, an arylene group, an ether bond, an ester bond, or a group in which two or more of these are bonded, and more preferably an alkylene group having 1 to 20 carbon atoms.

The specific resin 1 may have one kind of the constitutional unit represented by Formula (A1), or may have two or more kinds thereof. In addition, the specific resin 1 may have one kind of the constitutional unit represented by Formula (B1), or may have two or more kinds thereof. The content (in a case of including two or more kinds, total content) of the constitutional unit represented by Formula (A1) and the constitutional unit represented by Formula (B1) is preferably 1% by mass to 60% by mass, more preferably 5% by mass to 40% by mass, and still more preferably 5% to 20% by mass with respect to the total mass of the specific resin 1.

[Constitutional Unit D]

It is also preferable that the specific resin 1 has an ethylenically unsaturated bond-containing group and further includes a constitutional unit D which is different from the constitutional unit represented by Formula (A1) and the constitutional unit represented by Formula (B1).

[Constitutional Unit Represented by Formula (D1)]

The specific resin 1 preferably further includes a constitutional unit represented by Formula (D1) as the constitutional unit D.

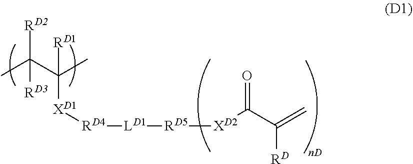

In Formula (D1), R^{D1} to R^{D3} each independently represent a hydrogen atom or an alkyl group,

X^{D1} represents —COO—, —CONR^{D6}—, or an arylene group, where R^{D6} represents a hydrogen atom, an alkyl group, or an aryl group,

R^{D4} represents a divalent linking group,

L^{D1} represents a group represented by Formula (D2), Formula (D3), or Formula (D3′),

R^{D5} represents an (nD+1)-valent linking group,

X^{D2} represents an oxygen atom or NR^{D7}—, where R^{D7} represents a hydrogen atom, an alkyl group, or an aryl group,

R^{D} represents a hydrogen atom or a methyl group, and

nD represents an integer of 1 or more,

where in a case where nD is 2 or more, two or more X^{D2}'s and two or more R^{D}'s may be the same or different from each other.

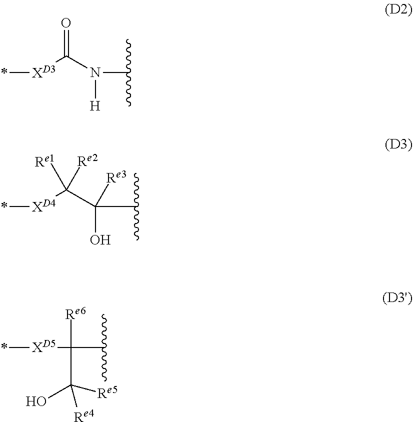

In Formulae (D2), (D3), and (D3′), X^{D3} represents an oxygen atom or —NH—,

X^{D4} represents an oxygen atom or COO—,

R^{e1} to R^{e3} each independently represent a hydrogen atom or an alkyl group, where at least two of R^{e1} to R^{e3} may be bonded to each other to form a ring structure,

X_{D5} represents an oxygen atom or —COO—,

R^{e4} to R^{e6} each independently represent a hydrogen atom or an alkyl group, where at least two of R^{e4} to R^{e6} may be bonded to each other to form a ring structure, and

* and a wavy line represent a bonding position with other structures.

The specific resin 1 may have one kind of the constitutional unit represented by Formula (D1), or may have two or more kinds thereof. The content of the constitutional unit represented by Formula (D1) is preferably 1% to 80% by mass, more preferably 1% to 70% by mass, and particularly preferably 1% to 60% by mass with respect to the total mass of the specific resin 1.

[Constitutional unit represented by Formula (D4)]

From the viewpoint of dispersion stability and developability, the specific resin 1 preferably further has a constitutional unit represented by Formula (D4).

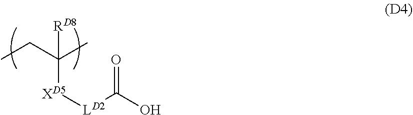

In Formula (D4), R^{D8} represents a hydrogen atom or an alkyl group, X^{D5} represents —COO—, —CONR^{B}—, or an arylene group, where R^{B} represents a hydrogen atom, an alkyl group, or an aryl group, and L^{D2} represents an aliphatic hydrocarbon group having 1 to 10 carbon atoms, an aromatic hydrocarbon group having 6 to 20 carbon atoms, or a group in which two or more groups selected from the group consisting of aliphatic hydrocarbon groups having 1 to 10 carbon atoms and aromatic hydrocarbon groups having 6 to 20 carbon atoms are bonded to one or more groups selected from the group consisting of ether bonds and ester bonds. Furthermore, in a case where X^{D5} is an arylene group, L^{D2} may be a single bond.

The specific resin 1 may have one kind of the constitutional unit represented by Formula (D4), or may have two or more kinds thereof. From the viewpoint of developability, formation of a pattern shape, and dispersion stability, the content of the constitutional unit represented by Formula (D4) is preferably 20% by mass to 80% by mass, more preferably 20% by mass to 70% by mass, and particularly preferably 20% by mass to 60% by mass with respect to the total mass of the specific resin 1.

[Constitutional Unit Represented by Formula (D5)]

From the viewpoint of dispersion stability, the specific resin 1 preferably has a constitutional unit represented by Formula (D5), and from the viewpoint of dispersion stability and developability, the specific resin more preferably further has the constitutional unit represented by Formula (D4) and a constitutional unit represented by Formula (D5).

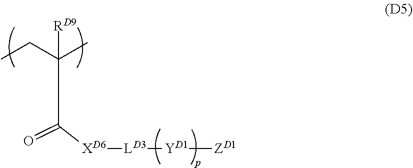

In Formula (D5), R^{D9} represents a hydrogen atom or an alkyl group,

X^{D6} represents an oxygen atom or NR^{C}—, where R^{C} represents a hydrogen atom, an alkyl group, or an aryl group.

L^{D3} represents a divalent linking group.

Y^{D1} represents an alkyleneoxy group or an alkylenecarbonyloxy group,

Z^{D1} represents an aliphatic hydrocarbon group having 1 to 20 carbon atoms or an aromatic hydrocarbon group having 6 to 20 carbon atoms, and

p represents an integer of 1 or more, where in a case where p is 2 or more, p pieces of Y^{D1}'s may be the same or different from each other.

The specific resin 1 may have one kind of the constitutional unit represented by Formula (D5), or may have two or more kinds thereof. From the viewpoint of developability and dispersion stability, the content of the constitutional unit represented by Formula (D5) is preferably 5% by mass to 80% by mass, more preferably 5% by mass to 70% by mass, and particularly preferably 5% by mass to 60% by mass with respect to the total mass of the specific resin 1.

[Other Constitutional Units]

The specific resin 1 may have a constitutional unit other than the above-described constitutional units represented by Formula (A1), Formula (B1), Formula (D1), Formula (D4), and Formula (D5). The other constitutional units are not particularly limited, and a known constitutional unit may be used.

(Specific Resin 2)

As the dispersant C1, it is also preferable to use a resin (hereinafter, also referred to as a specific resin 2) having a constitutional unit represented by Formula (1), a constitutional unit represented by Formula (2), and a constitutional unit represented by Formula (3).

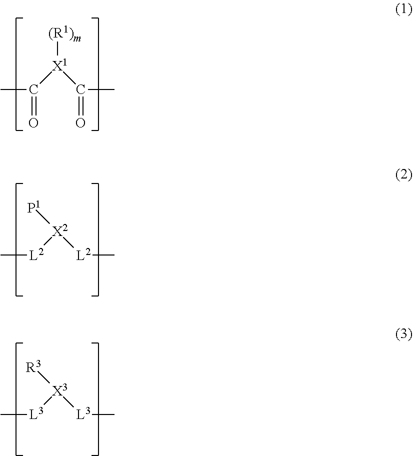

In Formulae (1) to (3), X¹ represents an (m+2)-valent organic group, X² and X³ represent a trivalent organic group, m represents an integer of 1 to 4, L²'s each independently represent O or NR, L³'s each independently represent a carbonyl group, O, or NR, R represents a hydrogen atom, an alkyl group, or an aryl group, P¹ represents a group having a polymer chain, R¹'s each independently represent a substituent, and R³ represents a group having an ethylenically unsaturated bond-containing group.

It is preferable that R's in Formula (1) are each independently an acid group or a salt of an acid group. As the above-described acid group, a carboxyl group, a sulfo group, or a phosphonic acid group is preferable, a carboxyl group or a sulfo group is more preferable, and a carboxyl group is particularly preferable.

A counter cation forming the salt in the above-described salt of the acid group is not particularly limited, but an alkali metal ion, an alkaline earth metal ion, or a primary to quaternary ammonium ion is preferable, an alkali metal ion or a quaternary ammonium ion is more preferable, and an alkali metal ion is particularly preferable. In addition, the counter cation may be a monovalent cation or a divalent or higher cation as long as the compound as a whole is electrically neutral, but a monovalent cation is preferable.

As R¹, from the viewpoint of developability, dispersion stability, pattern line width stability after leaving, and the like, a carboxyl group or a salt of a carboxyl group is particularly preferable.

m in Formula (1) is preferably an integer of 1 to 3, more preferably 1 or 2, and particularly preferably 2.

As X¹ in Formula (1), an (m+2)-valent organic group having an aliphatic ring or an aromatic ring is preferable, an (m+2)-valent organic group having an aromatic ring is more preferable, an (m+2)-valent hydrocarbon group having a cyclohexane ring structure or a benzene ring structure is still more preferable, and an (m+2)-valent hydrocarbon group having a benzene ring structure is particularly preferable. Preferred examples of the (m+2)-valent hydrocarbon group having an aliphatic ring structure or an aromatic ring structure include groups shown below. A wavy line portion represents a bonding position with the carbonyl group or R¹ in Formula (1).

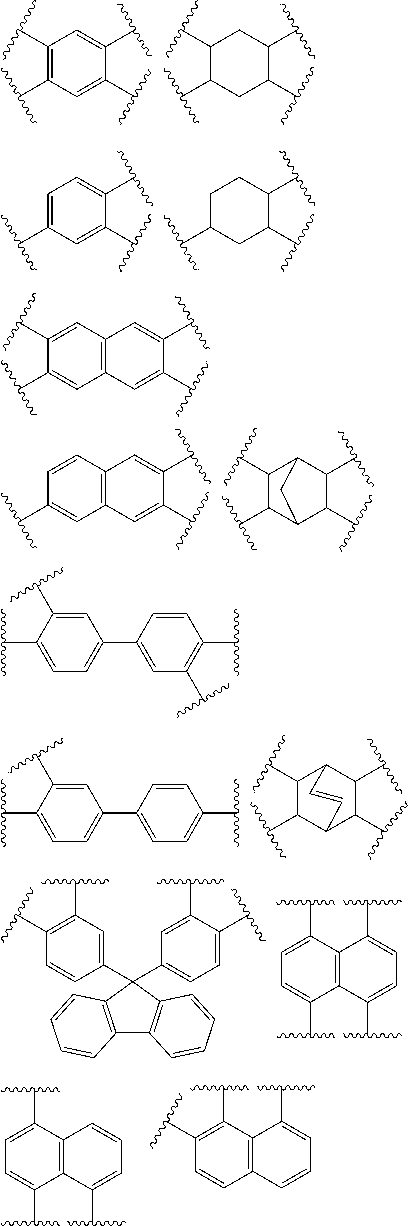

In addition, preferred examples of the constitutional unit represented by Formula (1) include a constitutional unit formed from an aromatic tricarboxylic acid anhydride and a constitutional unit formed from an aromatic tetracarboxylic acid anhydride. Specific examples of the aromatic tricarboxylic acid anhydride include a benzenetricarboxylic acid anhydride (1,2,3-benzenetricarboxylic acid anhydride, trimellitic acid anhydride [1,2,4-benzenetricarboxylic acid anhydride], and the like), a naphthalenetricarboxylic acid anhydride (1,2,4-naphthalenetricarboxylic acid anhydride, 1,4,5-naphthalenetricarboxylic acid anhydride, 2,3,6-naphthalenetricarboxylic acid anhydride, 1,2,8-naphthalenetricarboxylic acid anhydride, and the like), 3,4,4′-benzophenonetricarboxylic acid anhydride, 3,4,4′-biphenylethertricarboxylic acid anhydride, 3,4,4′-biphenyltricarboxylic acid anhydride, 2,3,2′-biphenyltricarboxylic acid anhydride, 3,4,4′-biphenylmethanetricarboxylic acid anhydride, and 3,4,4′-biphenylsulfonetricarboxylic acid anhydride. Specific examples of the aromatic tetracarboxylic acid anhydride include pyromellitic acid dianhydride, ethylene glycol dianhydrous trimellitic acid ester, propylene glycol dianhydrous trimellitic acid ester, butylene glycol dianhydrous trimellitic acid ester, 3,3′,4,4′-benzophenonetetracarboxylic acid dianhydride, 3,3′,4,4′-biphenylsulfonetetracarboxylic acid dianhydride. 1,4,5,8-naphthalenetetracarboxylic acid dianhydride, 2,3,6,7-naphthalenetetracarboxylic acid dianhydride, 3,3′,4,4′-biphenylethertetracarboxylic acid dianhydride, 3,3′,4,4′-dimethyldiphenylsilanetetracarboxylic acid dianhydride, 3,3′,4,4′-tetraphenylsilanetetracarboxylic acid dianhydride, 1,2,3,4-frantetracarboxylic acid dianhydride, 4,4′-bis(3,4-dicarboxyphenoxy)diphenylsulfide dianhydride, 4,4′-bis(3,4-dicarboxyphenoxy)diphenylsulfone dianhydride, 4,4′-bis(3,4-dicarboxyphenoxy)diphenylpropane dianhydride, 3,3′,4,4′-perfluoroisopropyridendiphthalic acid dianhydride, 3,3′,4,4′-biphenyltetracarboxylic acid dianhydride, bis(phthalic acid) phenylphosphineoxide dianhydride, p-phenylene-bis(triphenylphthalic acid) dianhydride, m-phenylene-bis(triphenylphthalic acid) dianhydride, bis(triphenylphthalic acid)-4,4′-diphenylether dianhydride, bis(triphenylphthalic acid)-4,4′-diphenylmethane dianhydride, 9,9-bis(3,4-dicarboxyphenyl)fluorene dianhydride, 9,9-bis[4-(3,4-dicarboxyphenoxy)phenyl]fluorene dianhydride, 3,4-dicarboxy-1,2,3,4-tetrahydro-1-naphthalenesuccinic acid dianhydride, and 3,4-dicarboxy-1,2,3,4-tetrahydro-6-methyl-1-naphthalenesuccinic acid dianhydride.

The specific resin 2 may have one kind of the above-described constitutional unit represented by Formula (1), or may have two or more kinds thereof. The content of the above-described constitutional unit represented by Formula (1) is preferably 0.1% by mass to 50% by mass, more preferably 1% by mass to 30% by mass, still more preferably 2% by mass to 20% by mass, and particularly preferably 6% by mass to 15% by mass with respect to the total mass of the specific resin 2.

L²'s in Formula (2) are each independently preferably O or NH and more preferably O. In addition, it is preferable that two L²'s in Formula (2) have the same group. X² in Formula (2) may be an aliphatic group, an aromatic group, or a group of a combination of these groups, but from the viewpoint of moisture resistance and adhesiveness of the cured product to be obtained, an aliphatic group is preferable. In addition, X² in Formula (2) is preferably a trivalent group having a sulfur atom, more preferably a trivalent group having a thioether bond, and particularly preferably a trivalent aliphatic group having a thioether bond. Further, the number of carbon atoms in X² in Formula (2) is preferably 1 to 30, more preferably 2 to 15, still more preferably 3 to 8, particularly preferably 3 to 6, and most preferably 3. Among these, from the viewpoint of pattern line width stability after leaving and the like, X² in Formula (2) is preferably a group represented by Formula (X-1) and more preferably a group represented by Formula (X-2).

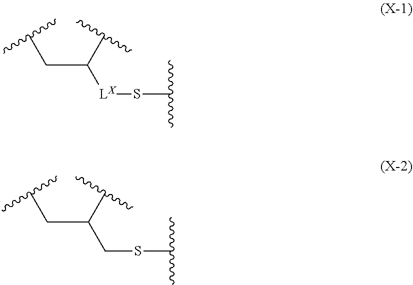

In Formulae (X-1) and (X-2), L^{X} represents an alkylene group having 1 to 8 carbon atoms, and a wavy line portion represents a bonding position with L² or P¹. In Formulae (X-1) and (X-2), it is preferable that P¹ in Formula (2) is bonded to the sulfur atom.

As P¹ in Formula (2), from the viewpoint of developability, dispersion stability, pattern line width stability after leaving, and the like, a group having an acrylic resin chain, a polyester chain, a polyether chain, or a polymer chain of a combination of two or more of these chains is preferable, a group having an acrylic resin chain, a polyester chain, or a polyether chain is more preferable, and a group having an acrylic resin chain is particularly preferable.

In addition, as the above-described acrylic resin chain, from the viewpoint of developability, dispersion stability, pattern line width stability after leaving, and the like, an acrylic resin chain obtained by copolymerizing two or more kinds of alkyl (meth)acrylate compounds is preferable, an acrylic resin chain obtained by copolymerizing n-butyl (meth)acrylate and another alkyl (meth)acrylate compound is more preferable, and an acrylic resin chain obtained by copolymerizing n-butyl (meth)acrylate and methyl (meth)acrylate or ethyl (meth)acrylate is particularly preferable.

From the viewpoint of developability, dispersion stability, pattern line width stability after leaving, and the like, the weight-average molecular weight of the polymer chain in P¹ in Formula (2) is preferably 500 to 20,000. The lower limit is more preferably 600 or more and still more preferably 1000 or more. The upper limit is more preferably 10000 or less, still more preferably 5000 or less, and particularly preferably 3000 or less.

The polymer chain in P¹ in Formula (2) and X² in Formula (2) may be bonded through a linking group or may be directly bonded, but it is preferable to be directly bonded. In addition, the number of atoms in the above-described linking group is preferably 1 to 30 and more preferably 2 to 20.

The polymer chain in P¹ in Formula (2) is preferably a polymer chain having a constitutional unit represented by Formulae (P-1) to (P-5), and more preferably a polymer chain having a constitutional unit represented by Formula (P-5).

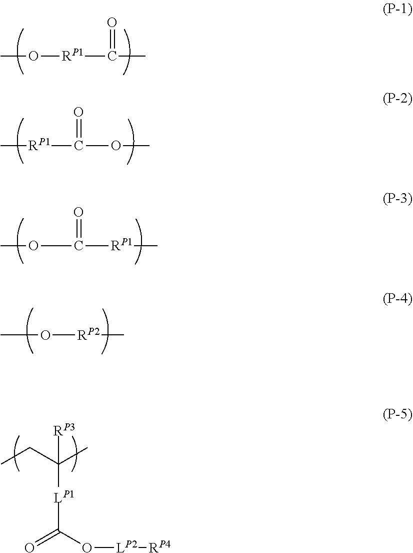

In the formulae, R^{P1} and R^{P2} each represent an alkylene group. As the alkylene group represented by R^{P1} and R^{P2}, a linear or branched alkylene group having 1 to 20 carbon atoms is preferable, a linear or branched alkylene group having 2 to 16 carbon atoms is more preferable, and a linear or branched alkylene group having 3 to 12 carbon atoms is still more preferable.

In the formulae, R^{P3} represents a hydrogen atom or a methyl group.

In the formulae, L^{P1} represents a single bond or an arylene group and L^{P2} represents a single bond or a divalent linking group. L^{P1} is preferably a single bond. Examples of the divalent linking group represented by L^{P2} include an alkylene group (preferably an alkylene group having 1 to 12 carbon atoms), an arylene group (preferably an arylene group having 6 to 20 carbon atoms). —NH—, —SO—, —SO₂—, —CO—, —O—, —COO—, —OCO—, —S—, —NHCO—, —CONH—, and a group formed by a combination of two or more these groups.

R^{P4} represents a hydrogen atom or a substituent. Examples of the substituent include a hydroxyl group, a carboxyl group, an alkyl group, an aryl group, a heteroaryl group, an alkoxy group, an aryloxy group, a heteroaryloxy group, an alkylthioether group, an arylthioether group, a heteroarylthioether group, and an ethylenically unsaturated bond-containing group.

In addition, the polymer chain in P¹ in Formula (2) also preferably has a constitutional unit having an acid group. Examples of the acid group include a carboxyl group, a phosphoric acid group, a sulfo group, and a phenolic hydroxyl group. According to this aspect, the dispersibility of the pigment in the composition can be further improved. Furthermore, developability can also be further improved. The content of the constitutional unit having an acid group is preferably 1% by mass to 30% by mass, more preferably 2% by mass to 20% by mass, and still more preferably 3% by mass to 10% by mass with respect to the total mass of the polymer chain.

The specific resin 2 may have one kind of the above-described constitutional unit represented by Formula (2), or may have two or more kinds thereof.

The content of the above-described constitutional unit represented by Formula (2) is preferably 50% e by mass to 98% by mass, more preferably 60% by mass to 95% by mass, and particularly preferably 70% by mass to 90% by mass with respect to the total mass of the specific resin 2.

In Formula (3), it is preferable that L³ is O or NR and X³ is a trivalent aliphatic group, it is more preferable that L³ is O or NH and X³ is a trivalent aliphatic group having a thioether bond, and it is particularly preferable that L³ is O.

In Formula (3), in a case where L³ is O or NR, a preferred aspect of X³ is the same as the preferred aspect of X² in Formula (2).

In addition, from the viewpoint of producing suitability, L³ in Formula (3) is preferably O or NR, more preferably O or NH, and particularly preferably O.

Further, the specific resin 2 preferably has both the constitutional unit represented by Formula (3), in which L³ is O or NR, and the constitutional unit represented by Formula (3), in which L³ is a carbonyl group.

In addition, in Formula (3), from the viewpoint of developability and dispersion stability, it is preferable that L³ is a carbonyl group and X³ is a trivalent organic group having an aromatic ring.

In Formula (3), in a case where L³ is a carbonyl group, a preferred aspect of X³ is the same as the preferred aspect of X¹ in a case where m in Formula (1) is 1 and X¹ is an (m+2)-valent hydrocarbon group having an aromatic ring structure.

Examples of the ethylenically unsaturated bond-containing group in the group having an ethylenically unsaturated bond-containing group, which is represented by R³ in Formula (3), include a vinyl group, a (meth)allyl group, a (meth)acryloyl group, and a vinylphenyl group. Among these, from the viewpoint of reactivity, a (meth)acryloyl group or a vinylphenyl group is preferable, and a (meth)acryloyl group is more preferable.

The number of ethylenically unsaturated bond-containing groups in R³ in Formula (3) is not particularly limited, but from the viewpoint of developability and curing properties, is preferably 1 to 10, more preferably 1 to 6, still more preferably 1 or 2, and particularly preferably 1.

In R³ of Formula (3), the ethylenically unsaturated bond-containing group may be directly bonded to X³ in Formula (3), or may be bonded thereto through a linking group. The number of carbon atoms in the above-described linking group is not particularly limited, but from the viewpoint of developability, dispersion stability, and pattern line width stability after leaving, it is preferable to be 1 to 40, more preferable to be 1 to 20, still more preferable to be 2 to 9, and particularly preferable to be 3 to 5. In addition, the above-described linking group is preferably an aliphatic group, and a divalent aliphatic hydrocarbon group or a group in which one or more divalent aliphatic hydrocarbon groups are bonded to one or more structures selected from the group consisting of an ether bond, an ester bond, an amide bond, a urethane bond, and a urea bond is preferable. Further, the above-described linking group may have a substituent such as a hydroxyl group and an amino group. Among these, preferred examples of the substituent include a hydroxyl group.

The molecular weight of the above-described constitutional unit represented by Formula (3) is preferably 100 to 1000, more preferably 100 to 7M), and still more preferably 100 to 500.

The specific resin 2 may have one kind of the above-described constitutional unit represented by Formula (3), or may have two or more kinds thereof. The content of the above-described constitutional unit represented by Formula (3) is preferably 0.1% by mass to 50% by mass, more preferably 0.5% by mass to 20% by mass, still more preferably 1% by mass to 15% by mass, and particularly preferably 2% by mass to 10% by mass with respect to the total mass of the specific resin 2.

The specific resin 2 may have a constitutional unit other than the constitutional units represented by Formulae (1) to (3). The other constitutional units are not particularly limited, and examples thereof include a constitutional unit formed from a polyvalent carboxylic acid compound, a polyhydric alcohol compound, a polyvalent amine compound, a hydroxycarboxylic acid compound, a polyvalent isocyanate compound, or the like. The specific resin 2 may have one kind of the above-described other constitutional unit, may have two or more kinds of the above-described other constitutional units, or may not have the above-described other constitutional units. The total content of the constitutional unit represented by Formulae (1) to (3) is preferably 50% by mass or more, more preferably 80% by mass or more, still more preferably 90% by mass or more, and particularly preferably 95% by mass to 100% by mass with respect to the total mass of the specific resin 2.

[Dispersant not Including Ethylenically Unsaturated Bond-Containing Group (Dispersant C2)]

The dispersant C contained in the photosensitive composition according to the embodiment of the present invention may contain a dispersant (hereinafter, also referred to as a dispersant C2) not including an ethylenically unsaturated bond-containing group.

Examples of the dispersant C2 include an acidic dispersant (acidic resin) and a basic dispersant (basic resin). Here, the acidic dispersant (acidic resin) represents a resin in which the amount of the acid group is larger than the amount of the basic group. The acidic dispersant (acidic resin) is preferably a resin in which the amount of the acid group occupies 70 mol % or more in a case where the total amount of the acid group and the basic group is 100 mol %, and more preferably a resin substantially consisting of only an acid group. The acid group included in the acidic dispersant (acidic resin) is preferably a carboxyl group. The acid value of the acidic dispersant (acidic resin) is preferably 40 to 105 mgKOH/g, more preferably 50 to 105 mgKOH/g, and still more preferably 60 to 105 mgKOH/g. In addition, the basic dispersant (basic resin) represents a resin in which the amount of the basic group is larger than the amount of the acid group. The basic dispersant (basic resin) is preferably a resin in which the amount of the basic group is more than 50 mol % in a case where the total amount of the acid group and the basic group is 100 mol %. The basic group included in the basic dispersant is preferably an amino group.

The dispersant C2 is also preferably a graft resin. With regard to details of the graft resin, reference can be made to the description in paragraph Nos. 0025 to 0094 of JP2012-255128A, the contents of which are incorporated herein by reference.

The dispersant C2 is also preferably a polyimine-based dispersant including a nitrogen atom in at least one of the main chain or the side chain. As the polyimine-based dispersant, a resin having a main chain which has a partial structure having a functional group of pKa 14 or less, and a side chain which has 40 to 10000 atoms, in which at least one of the main chain or the side chain has a basic nitrogen atom, is preferable. The basic nitrogen atom is not particularly limited as long as it is a nitrogen atom exhibiting basicity. With regard to the polyimine-based dispersant, reference can be made to the description in paragraph Nos. 0102 to 0166 of JP2012-255128A, the contents of which are incorporated herein by reference.

The dispersant C2 is also preferably a resin having a structure in which a plurality of polymer chains are bonded to a core portion. Examples of such a resin include dendrimers (including star polymers). In addition, specific examples of the dendrimer include polymer compounds C-1 to C-31 described in paragraph Nos. 0196 to 0209 of JP2013-043962A.

The dispersant C2 is also preferably a resin (hereinafter, also referred to as a resin Ac) having an aromatic carboxyl group. The resin Ac may include the aromatic carboxyl group in the main chain of the repeating unit, or in the side chain of the repeating unit, but it is preferable that the aromatic carboxyl group is included in the main chain of the repeating unit. In the aromatic carboxyl group, the number of carboxyl groups bonded to an aromatic ring is preferably 1 to 4 and more preferably 1 or 2.

The resin Ac is preferably a resin including at least one repeating unit selected from a repeating unit represented by Formula (b-101) or a repeating unit represented by Formula (b-110).

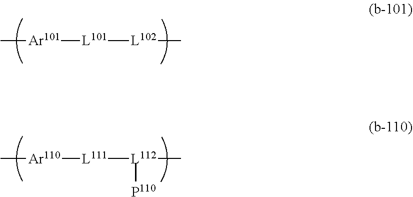

In Formula (b-101), Ar¹⁰¹ represents a group including an aromatic carboxyl group, L¹⁰¹ represents —COO— or —CONH—, and L¹⁰² represents a divalent linking group.

In Formula (b-110), Ar¹¹⁰ represents a group including an aromatic carboxyl group, L¹¹¹ represents —COO— or —CONH—, L¹¹² represents a trivalent linking group, and P¹¹⁰ represents a polymer chain.

Specific examples of the resin Ac include compounds described in JP2017-156652A, the contents of which are incorporated herein by reference.

A commercially available product is also available as the dispersant C2, and specific examples thereof include DISPERBYK series (for example, DISPERBYK-11, 161, and the like) manufactured by BYK Chemie, and Solsperse series (for example, Solsperse 76500) manufactured by Lubrizol Corporation. The dispersing agents described in paragraph Nos. 0041 to 0130 of JP2014-130338A can also be used, the contents of which are incorporated herein by reference. In addition, as the dispersant C2, dispersants described in JP2018-150498A, JP2017-100116A, JP2017-100115A, JP2016-108520A, JP2016-108519A, and JP2015-232105A may be used.

A content of the dispersant C in the total solid content of the photosensitive composition is preferably 5% to 30% by mass. The lower limit is preferably 10% by mass or more and more preferably 12.5% by mass or more. The upper limit is preferably 25% by mass or less and more preferably 20% by mass or less.

In addition, the content of the dispersant C is preferably 5 to 50 parts by mass with respect to 100 parts by mass of the coloring material A. The lower limit is preferably 10 parts by mass or more and more preferably 15 parts by mass or more. The upper limit is preferably 40 parts by mass or less and more preferably 30 parts by mass or less.

In addition, the content of the dispersant C is preferably 50 to 1500 parts by mass with respect to 1M parts by mass of the pigment derivative D. The lower limit is preferably 100 parts by mass or more. The upper limit is preferably 1000 parts by mass or less and more preferably 500 parts by mass or less.

In addition, a content of the dispersant C1 in the dispersant C is preferably 30% to 100% by mass, more preferably 40% to 100% by mass, and still more preferably 50% to 100% by mass.

<<Polymerizable Monomer>>

The photosensitive composition according to the embodiment of the present invention preferably contains a polymerizable monomer. As the polymerizable monomer, a known compound which is cross-linkable by a radical, an acid, or heat can be used. The polymerizable monomer is preferably, for example, a compound having an ethylenically unsaturated bond-containing group. Examples of the ethylenically unsaturated bond-containing group include a vinyl group, a (meth)allyl group, and a (meth)acryloyl group. The polymerizable monomer used in the present invention is preferably a radically polymerizable monomer.

A molecular weight of the polymerizable monomer is preferably 100 to 3000. The upper limit is more preferably 2000 or less and still more preferably 1500 or less. The lower limit is more preferably 150 or more and still more preferably 250 or more.

The polymerizable monomer is preferably a compound including 3 or more ethylenically unsaturated bond-containing groups, more preferably a compound including 3 to 15 ethylenically unsaturated bond-containing groups, and still more preferably a compound including 3 to 6 ethylenically unsaturated bond-containing groups. In addition, the polymerizable monomer is preferably a 3- to 15-functional (meth)acrylate compound and more preferably a 3- to 6-functional (meth)acrylate compound. Specific examples of the polymerizable monomer include the compounds described in paragraph Nos. 0095 to 0108 of JP2009-288705A, paragraph No. 0227 of JP2013-029760A, paragraph Nos. 0254 to 0257 of JP2008-292970A, paragraph Nos. 0034 to 0038 of JP2013-253224A, paragraph No. 0477 of JP2012-20844A JP2017-48367A, JP6057891B, JP6031807B, and JP2017-194662A, the contents of which are incorporated herein by reference.

As the polymerizable monomer, dipentaerythritol tri(meth)acrylate (as a commercially available product, KAYARAD D-330 manufactured by Nippon Kayaku Co., Ltd.), dipentaerythritol tetra(meth)acrylate (as a commercially available product, KAYARAD D-320 manufactured by Nippon Kayaku Co., Ltd.), dipentaerythritol penta(meth)acrylate (as a commercially available product, KAYARAD D-310 manufactured by Nippon Kayaku Co., Ltd.), dipentaerythritol hexa(meth)acrylate (as a commercially available product, KAYARAD DPHA manufactured by Nippon Kayaku Co., Ltd., NK ESTER A-DPH-12E manufactured by Shin-Nakamura Chemical Co., Ltd.), or a compound having a structure in which these (meth)acryloyl groups are bonded through an ethylene glycol and/or a propylene glycol residue (for example, SR454 and SR499 which are commercially available products from Sartomer) is preferable. In addition, as the polymerizable monomer, diglycerin ethylene oxide (EO)-modified (meth)acrylate (as a commercially available product, M-460 manufactured by TOAGOSEI CO., LTD.), pentaerythritol tetmacrylate (NK ESTER A-TMMT manufactured by Shin-Nakamura Chemical Co., Ltd.), 1,6-hexanediol diacrylate (KAYARAD HDDA manufactured by Nippon Kayaku Co., Ltd.). RP-1040 (manufactured by Nippon Kayaku Co., Ltd.). ARONIX TO-2349 (manufactured by TOAGOSEI CO., LTD.), NK OLIGO UA-7200 (manufactured by Shin-Nakamura Chemical Co., Ltd.), 8UH-1006 and 8UH-1012 (manufactured by Taisei Fine Chemical Co., Ltd.), Light Acrylate POB-A0 (manufactured by KYOEISHA CHEMICAL Co., Ltd.), and the like can also be used.

As the polymerizable monomer, it is also preferable to use a trifunctional (meth)acrylate compound such as trimethylolpropane tri(meth)acrylate, trimethylolpropane propyleneoxide-modified tri(meth)acrylate, trimethylolpropane ethyleneoxide-modified tri(meth)acrylate, isocyanuric acid ethyleneoxide-modified tri(meth)acrylate, and pentaerythritol tri(meth)acrylate. Examples of a commercially available product of the trifunctional (meth)acrylate compound include ARONIX M-309, M-310, M-321, M-350, M-360, M-313, M-315, M-306, M-305, M-303, M-452, and M-450 (manufactured by TOAGOSEI CO., LTD.), NK ESTER A9300, A-GLY-9E, A-GLY-20E, A-TMM-3, A-TMM-3L, A-TMM-3LM-N, A-TMPT, and TMPT (manufactured by Shin-Nakamura Chemical Co., Ltd.), and KAYARAD GPO-303, TMPTA, THE-330, TPA-330, and PET-30 (manufactured by Nippon Kayaku Co., Ltd.).

As the polymerizable monomer, a polymerizable monomer having an acid group can also be used. By using a polymerizable monomer having an acid group, a photosensitive composition in a non-exposed portion is easily removed during development and the generation of the development residue can be suppressed. Examples of the acid group include a carboxyl group, a sulfo group, and a phosphoric acid group, and a carboxyl group is preferable. Examples of a commercially available product of the polymerizable monomer having an acid group include ARONIX M305, M-510, M-520, and ARONIX TO-2349 (manufactured by TOAGOSEI CO., LTD). An acid value of the polymerizable monomer having an acid group is preferably 0.1 to 40 mgKOH/g and more preferably 5 to 30 mgKOH/g.

As the polymerizable monomer, a polymerizable monomer having a caprolactone structure can also be used. Examples of the polymerizable monomer having a caprolactone structure include DPCA-20, DPCA-30, DPCA-60, and DPCA-120, each of which is commercially available as KAYARAD DPCA series from Nippon Kayaku Co., Ltd.

As the polymerizable monomer, a polymerizable monomer having an alkyleneoxy group can also be used. The polymerizable monomer having an alkyleneoxy group is preferably a polymerizable monomer having an ethyleneoxy group and/or a propyleneoxy group, more preferably a polymerizable monomer having an ethyleneoxy group, and still more preferably a trifunctional to hexafunctional (meth)acrylate compound having 4 to 20 ethyleneoxy groups. Examples of a commercially available product of the polymerizable monomer having an alkyleneoxy group include SR-494 (manufactured by Sartomer), which is a tetrafunctional (meth)acrylate having 4 ethyleneoxy groups, and KAYARAD TPA-330 (manufactured by Nippon Kayaku Co., Ltd.), which is a trifunctional (meth)acrylate having 3 isobutyleneoxy groups.

As the polymerizable monomer, a polymerizable monomer having a fluorene skeleton can also be used. Examples of a commercially available product of the polymerizable monomer having a fluorene skeleton include OGSOL EA-0200 and EA-0300 (manufactured by Osaka Gas Chemicals Co., Ltd., a (meth)acrylate monomer having a fluorene skeleton).

As the polymerizable monomer, it is also preferable to use a compound which does not substantially include environmentally regulated substances such as toluene. Examples of a commercially available product of such a compound include KAYARAD DPHA LT and KAYARAD DPEA-12 LT (manufactured by Nippon Kayaku Co., Ltd.).

The urethane acrylates described in JP1973-041708B (JP-S48-041708B), JP1976-037193A (JP-S51-037193A), JP1990-032293B (JP-H02-032293B), or JP1990-016765B (JP-H02-016765B), or the urethane compounds having an ethylene oxide skeleton described in JP1983-049860B (JP-S58-049860B), JP1981-017654B (JP-S56-017654B), JP1987-039417B (JP-S62-039417B), or JP1987-039418B (JP-S62-039418B) are also suitable as the polymerizable monomer. In addition, the polymerizable monomers having an amino structure or a sulfide structure in the molecule, described in JP1988-277653A (JP-S63-277653A), JP1988-260909A (JP-S63-260909A), or JP1989-105238A (JP-1101-105238A), are also preferably used. In addition, as the polymerizable monomer, commercially available products such as UA-7200 (manufactured by Shin-Nakamura Chemical Co., Ltd.), DPHA-40H (manufactured by Nippon Kayaku Co., Ltd.), and UA-306H, UA-306T, UA-3061, AH-600, T-600, AI-600, and LINC-202UA (manufactured by KYOEISHA CHEMICAL Co., Ltd.) can also be used.

A content of the polymerizable monomer in the total solid content of the photosensitive composition is preferably 0.1% to 20% by mass. The lower limit is preferably 0.5% by mass or more and more preferably 1% by mass or more. The upper limit is preferably 10% by mass or less, more preferably 7.5% by mass or less, and still more preferably 5% by mass or less. The polymerizable monomer may be used singly or in combination of two or more kinds thereof. In a case where two or more kinds thereof are used, the total amount thereof is preferably within the above-described range.

<<Photopolymerization Initiator>>

The photosensitive composition according to the embodiment of the present invention preferably contains a photopolymerization initiator. The photopolymerization initiator is not particularly limited, and can be appropriately selected from known photopolymerization initiators. For example, a compound having photosensitivity to light in a range from an ultraviolet range to a visible range is preferable. The photopolymerization initiator is preferably a photoradical polymerization initiator.

Examples of the photopolymerization initiator include a halogenated hydrocarbon derivative (for example, a compound having a triazine skeleton or a compound having an oxadiazole skeleton), an acylphosphine compound, a hexaarylbiimidazole, an oxime compound, an organic peroxide, a thio compound, a ketone compound, an aromatic onium salt, an α-hydroxyketone compound, and an α-aminoketone compound. From the viewpoint of exposure sensitivity, as the photopolymerization initiator, a trihalomethyltriazine compound, a benzyldimethylketal compound, an α-hydroxyketone compound, an α-aminoketone compound, an acylphosphine compound, a phosphine oxide compound, a metallocene compound, an oxime compound, a triarylimidazole dimer, an onium compound, a benzothiazole compound, a benzophenone compound, an acetophenone compound, a cyclopentadiene-benzene-iron complex, a halomethyl oxadiazole compound, or a 3-aryl-substituted coumarin compound is preferable, a compound selected from an oxime compound, an α-hydroxyketone compound, an α-aminoketone compound, or an acylphosphine compound is more preferable, and an oxime compound is still more preferable. In addition, as the photopolymerization initiator, compounds described in paragraphs 0065 to 0111 of JP2014-130173A, compounds described in JP6301489B, peroxide-based photopolymerization initiators described in MATERIAL STAGE, p. 37 to 60, vol. 19, No. 3, 2019, photopolymerization initiators described in WO2018/221177A, photopolymerization initiators described in WO2018/110179A, photopolymerization initiators described in JP2019-043864A, and photopolymerization initiators described in JP2019-044030A, the contents of which are incorporated herein by reference.

Examples of a commercially available product of the α-hydroxyketone compound include Omnirad 184, Omnirad 1173, Omnirad 2959, and Omnirad 127 (all of which are manufactured by IGM Resins B.V.), Irgacure 184, Irgacure 1173, Irgacure 2959, and Irgacure 127 (all of which are manufactured by BASF). Examples of a commercially available product of the α-aminoketone compound include Omnirad 907, Omnirad 369, Omnirad 369E, and Omnirad 379EG (all of which are manufactured by IGM Resins B.V.), Irgacure 907, Irgacure 369, Irgacure 369E, and Irgacure 379EG (all of which are manufactured by BASF). Examples of a commercially available product of the acylphosphine compound include Omnirad 819 and Omnirad TPO (both of which are manufactured by IGM Resins B.V.), Irgacure 819 and Irgacure TPO (both of which are manufactured by BASF).

Examples of the oxime compound include the compounds described in JP2001-233842A, the compounds described in JP2000-080068A, the compounds described in JP2006-342166A, the compounds described in J. C. S. Perkin 11 (1979, pp. 1653 to 1660), the compounds described in J. C. S. Perkin 11(1979, pp. 156 to 162), the compounds described in Journal of Photopolymer Science and Technology (1995, pp. 202 to 232), the compounds described in JP2000-066385A, the compounds described in JP2004-534797A, the compounds described in JP2006-342166A, the compounds described in JP2017-019766A, the compounds described in JP6065596B, the compounds described in WO2015/152153A, the compounds described in WO2017/051680A, the compounds described in JP2017-198865A, the compounds described in paragraph Nos. 0025 to 0038 of WO2017/164127A, and compounds described in WO2013/167515A. Specific examples of the oxime compound include 3-benzoyloxyiminobutane-2-one, 3-acetoxyiminobutane-2-one, 3-propionyloxyiminobutane-2-one, 2-acetoxyiminopentane-3-one, 2-acetoxyimino-1-phenylpropane-1-one, 2-benzoyloxyimino-1-phenylpropane-1-one, 3-(4-toluene sulfonyloxy)iminobutane-2-one, and 2-ethoxycarbonyloxyimino-1-phenylpropane-1-one. Examples of a commercially available product thereof include Irgacure OXE01, Irgacure OXE02, Irgacure OXE03, and Irgacure OXE04 (all of which are manufactured by BASF), TR-PBG-304 (manufactured by TRONLY), and ADEKA OPTOMER N-1919 (manufactured by ADEKA Corporation; photopolymerization initiator 2 described in JP2012-014052A). In addition, as the oxime compound, it is also preferable to use a compound having no colorability or a compound having high transparency and being resistant to discoloration. Examples of a commercially available product include ADEKA ARKLS NCI-730, NCI-831, and NCI-930 (all of which are manufactured by ADEKA Corporation).

An oxime compound having a fluorene ring can also be used as the photopolymerization initiator. Specific examples of the oxime compound having a fluorene ring include the compounds described in JP2014-137466A.

As the photopolymerization initiator, an oxime compound having a skeleton in which at least one benzene ring of a carbazole ring is a naphthalene ring can also be used. Specific examples of such an oxime compound include the compounds described in WO2013/083505A.

An oxime compound having a fluorine atom can also be used as the photopolymerization initiator. Specific examples of the oxime compound having a fluorine atom include the compounds described in JP2010-262028A, the compounds 24, and 36 to 40 described in JP2014-500852A, and the compound (C-3) described in JP2013-164471A.

An oxime compound having a nitro group can be used as the photopolymerization initiator. The oxime compound having a nitro group is also preferably used in the form of a dimer. Specific examples of the oxime compound having a nitro group include the compounds described in paragraph Nos. 0031 to 0047 of JP2013-114249A and paragraph Nos. 0008 to 0012 and 0070 to 0079 of JP2014-137466A, the compounds described in paragraph Nos. 0007 to 0025 of JP4223071B, and ADEKA ARKLS NCI-831 (manufactured by ADEKA Corporation).

An oxime compound having a benzofuran skeleton can also be used as the photopolymerization initiator. Specific examples thereof include OE-01 to OE-75 described in WO2015/036910A.

In the present invention, as the photopolymerization initiator, an oxime compound in which a substituent having a hydroxyl group is bonded to a carbazole skeleton can also be used. Examples of such a photopolymerization initiator include compounds described in WO2019/089055A.

Specific examples or the oxime compound which are preferably used in the present invention are shown below, but the present invention is not limited thereto.

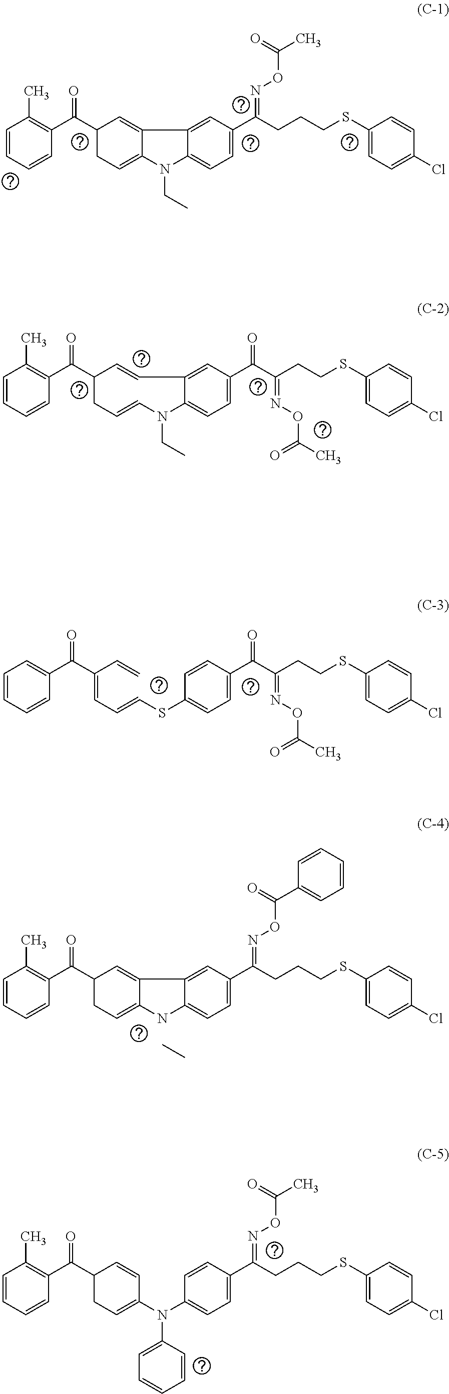

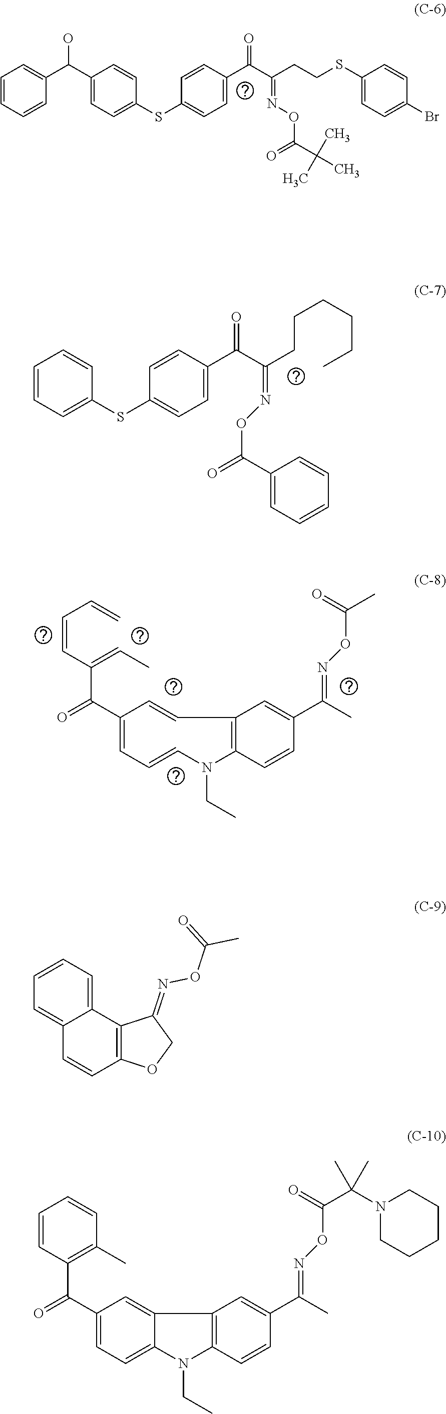 [SMILES: CC(C)(C)C(=O)O/N=C(\CCSc1ccc(Br)cc1)C(=O)c1ccc(Sc2ccc(C(O)c3ccccc3)cc2)cc1 ; CCCCCC/C(=N/OC(=O)c1ccccc1)C(=O)c1ccc(Sc2ccccc2)cc1 ; C=C/C=C\C(=C\C)C(=O)c1cccn(CC)c2ccc(/C(C)=N/OC(C)=O)cc2cc1 ; CC(=O)O/N=C1\COc2ccc3ccccc3c21 ; CCn1c2ccc(C(=O)c3ccccc3C)cc2c2cc(/C(C)=N/OC(=O)C(C)(C)N3CCCCC3)ccc21]

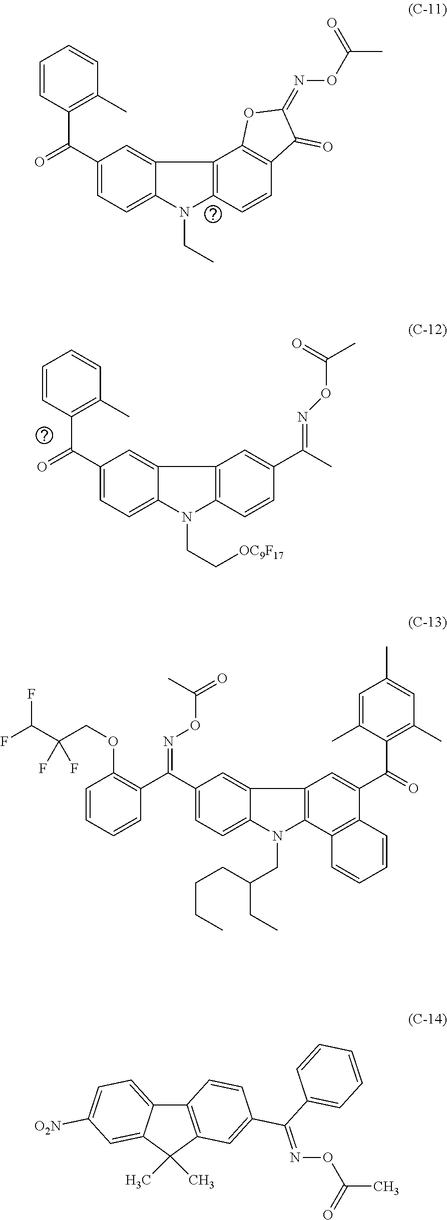

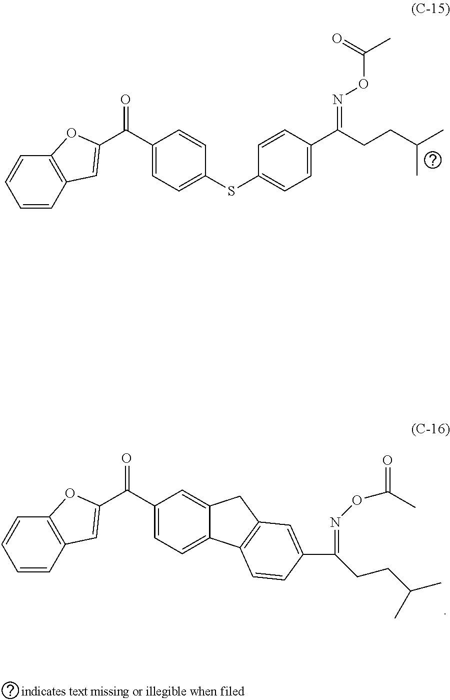 [SMILES: CC(=O)O/N=C(\CCC(C)C)c1ccc(Sc2ccc(C(=O)c3cc4ccccc4o3)cc2)cc1 ; CC(=O)O/N=C(\CCC(C)C)c1ccc2c(c1)Cc1cc(C(=O)c3cc4ccccc4o3)ccc1-2]

The oxime compound is preferably a compound having a maximal absorption wavelength in a wavelength range of 350 to 500 nm and more preferably a compound having a maximal absorption wavelength in a wavelength range of 360 to 480 nm. In addition, from the viewpoint of sensitivity, a molar absorption coefficient of the oxime compound at a wavelength of 365 nm or 405 nm is preferably high, more preferably 1000 to 300000, still more preferably 2000 to 300000, and particularly preferably 5000 to 200000. The molar absorption coefficient of a compound can be measured using a known method. For example, the molar absorption coefficient is preferably measured by a spectrophotometer (Cary-5 spectrophotometer, manufactured by Varian) using an ethyl acetate solvent at a concentration of 0.01 g/L.

As the photopolymerization initiator, a bifunctional or tri- or higher functional photoradical polymerization initiator may be used. By using such a photoradical polymerization initiator, two or more radicals are generated from one molecule of the photoradical polymerization initiator, and as a result, good sensitivity is obtained. In addition, in a case of using a compound having an asymmetric structure, crystallinity is reduced so that solubility in a solvent or the like is improved, precipitation is to be difficult over time, and temporal stability of the photosensitive composition can be improved. Specific examples of the bifunctional or tri- or higher functional photoradical polymerization initiator include dimers of the oxime compounds described in JP2010-527339A, JP2011-524436A, WO2015/004565A, paragraph Nos. 0407 to 0412 of JP2016-532675A, and paragraph Nos. 0039 to 0055 of WO2017/033680A; the compound (E) and compound (G) described in JP2013-522445A; Cmpd 1 to 7 described in WO2016/034963A; the oxime ester photoinitiators described in paragraph No. 0007 of JP2017-523465A; the photoinitiators described in paragraph Nos. 0020 to 0033 of JP2017-167399A; the photopolymerization initiator (A) described in paragraph Nos. 0017 to 0026 of JP2017-151342A; and the oxime ester photoinitiators described in JP6469669B.

A content of the photopolymerization initiator in the total solid content of the photosensitive composition is preferably 0.1% to 20% by mass. The lower limit is preferably 0.5% by mass or more and more preferably 1% by mass or more. The upper limit is preferably 10% by mass or less, more preferably 7.5% by mass or less, and still more preferably 5% by mass or less. The photopolymerization initiator may be used singly or in combination of two or more kinds thereof. In a case where two or more kinds thereof are used in combination, the total thereof is preferably within the above-described range.

<<Binder Resin>>

The photosensitive composition according to the embodiment of the present invention can further contain a binder resin. A weight-average molecular weight (Mw) of the binder resin is preferably 2000 to 2000000. The upper limit is preferably 1000000 or less and more preferably 500000 or less. The lower limit is preferably 3000 or more, more preferably 4000 or more, and still more preferably 5000 or more.

Examples of the binder resin include a (meth)acrylic resin, a (meth)acrylamide resin, an epoxy resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamideimide resin, a polyolefin resin, a cyclic olefin resin, a polyester resin, a styrene resin, and a siloxane resin.

The binder resin is also preferably a resin having an acid group. Examples of the acid group include a carboxyl group, a phosphoric acid group, a sulfo group, and a phenolic hydroxyl group. The resin having an acid group can also be used as an alkali-soluble resin. An acid value of the resin having an acid group is preferably 30 to 500 mgKOH/g. The lower limit is more preferably 50 mgKOH/g or more and still more preferably 70 mgKOH/g or more. The upper limit is more preferably 400 mgKOH/g or less, still more preferably 200 mgKOH/g or less, even still more preferably ISO mgKOH/g or less, and most preferably 120 mgKOH/g or less.

The resin having an acid group may have a repeating unit derived from a maleimide compound. Examples of the maleimide compound include N-alkylmaleimide and N-arylmaleimide. Examples of the repeating unit derived from a maleimide compound include a repeating unit represented by Formula (C-mi).

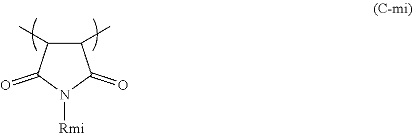

In Formula (C-mi), Rmi represents an alkyl group or an aryl group. The alkyl group preferably has 1 to 20 carbon atoms. The alkyl group may be linear, branched, or cyclic. The aryl group preferably has 6 to 20 carbon atoms, more preferably has 6 to 15 carbon atoms, and still more preferably has 6 to 10 carbon atoms. Rmi is preferably an aryl group.

As the binder resin, a resin including a repeating unit derived from a compound represented by Formula (ED1) and/or a compound represented by Formula (ED2) (hereinafter, these compounds will also be referred to as an “ether dimer”) is also preferable.

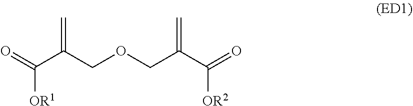

In Formula (ED1), R¹ and R² each independently represent a hydrogen atom or a hydrocarbon group having 1 to 25 carbon atoms, which may have a substituent.

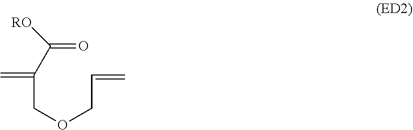

In Formula (ED2), R represents a hydrogen atom or an organic group having 1 to 30 carbon atoms. Specific examples of Formula (ED2) can be found in the description of JP2010-168539A.

Specific examples of the ether dimer can be found in paragraph No. 0317 of JP2013-029760A, the contents of which are incorporated herein by reference.

The binder resin is also preferably a resin including a repeating unit having a polymerizable group. Examples of the polymerizable group include ethylenically unsaturated bond-containing groups such as a vinyl group, a (meth)allyl group, and a (meth)acryloyl group.

The binder resin also preferably includes a resin including a repeating unit derived from a compound represented by Formula (III).

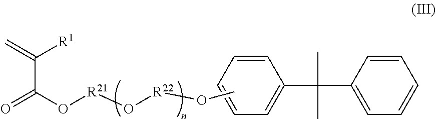

In the formula, R¹ represents a hydrogen atom or a methyl group. R²¹ and R²² each independently represent an alkylene group, and n represents an integer of 0 to 15. The number of carbon atoms in the alkylene group represented by R²¹ and R²² is preferably 1 to 10, more preferably 1 to 5, still more preferably 1 to 3, and particularly preferably 2 or 3. n is preferably an integer of 0 to 5, more preferably an integer of 0 to 4, and still more preferably an integer of 0 to 3.

Examples of the compound represented by Formula (III) include ethylene oxide- or propylene oxide-modified (meth)acrylate of para-cumylphenol. Examples of a commercially available product thereof include ARONIX M-110 (manufactured by TOAGOSEI CO., LTD.).

A content of the binder resin in the total solid content of the photosensitive composition is preferably 0.01% to 40% by mass. The upper limit is preferably 35% by mass or less and more preferably 30% by mass or less. The lower limit is preferably 0.5% by mass or more and more preferably 1% by mass or more.

In addition, the total content of the dispersant and the binder resin in the total solid content of the photosensitive composition is preferably 5% to 45% by mass. The upper limit is preferably 40% by mass or less and more preferably 35% by mass or less. The lower limit is preferably 7.5% by mass or more and more preferably 10% by mass or more.

In addition, in a case where the photosensitive composition according to the embodiment of the present invention contains the binder resin and the polymerizable monomer, the total content of the dispersant, the binder resin, and the polymerizable monomer in the total solid content of the photosensitive composition is preferably 5% to 45% by mass. The upper limit is preferably 40% by mass or less and more preferably 35% by mass or less. The lower limit is preferably 7.5% by mass or more and more preferably 10% by mass or more.

In addition, in a case where the photosensitive composition according to the embodiment of the present invention contains the binder resin and the polymerizable monomer, it is preferable to contain 0.5 to 100 parts by mass of the polymerizable monomer with respect to 100 parts by mass of the binder resin. The upper limit is preferably 90 parts by mass or less and more preferably 80 parts by mass or less. The lower limit is preferably 1 part by mass or more and more preferably 1.5 parts by mass or more.

<<Compound Having Cyclic Ether Group>>

The photosensitive composition can contain a compound having a cyclic ether group. Examples of the cyclic ether group include an epoxy group and an oxetanyl group. It is preferable that the compound having a cyclic ether group is a compound having an epoxy group (hereinafter, also referred to as an “epoxy compound”). Examples of the epoxy compound include a compound having one or more epoxy groups in one molecule, and a compound having two or more epoxy groups in one molecule is preferable. The epoxy compound is preferably a compound having 1 to 100 epoxy groups in one molecule. The upper limit of the number of epoxy groups included in the epoxy compound may be, for example, 10 or less or 5 or less. The lower limit of the epoxy group included in the epoxy compound is preferably 2 or more. As the epoxy compound, the compounds described in paragraph Nos. 0034 to 0036 of JP2013-011869A, paragraph Nos. 0147 to 0156 of JP2014-043556A, and paragraph Nos. 0085 to 0092 of JP2014-089408A, and the compounds described in JP2017-179172A can also be used. The contents of the publications are incorporated herein by reference.

The epoxy compound may be a low-molecular-weight compound (for example, having a molecular weight of less than 2000, and further, a molecular weight of less than 1000) or a high-molecular-weight compound (macromolecule) (for example, having a molecular weight of 1000 or more, and in a case of a polymer, having a weight-average molecular weight of 1000 or more). The weight-average molecular weight of the compound having an epoxy group is preferably 200 to 100000 and more preferably 500 to 5000). The upper limit of the weight-average molecular weight is still more preferably 10000 or less, particularly preferably 5000 or less, and even more preferably 3000 or less.

Examples of a commercially available product of the compound having a cyclic ether group include EHPE 3150 (manufactured by Daicel Corporation), EPICLON N-695 (manufactured by DIC Corporation), and MARPROOF G-0150M, G-0105SA, G-0130SP, G-0250SP, G-1005S, G-1005SA, G-1010S, G-2050M, G-01100, and G-01758 (all of which are manufactured by NOF Corporation, an epoxy group-containing polymer).

In a case where the photosensitive composition according to the embodiment of the present invention contains a compound having a cyclic ether group, a content of the compound having a cyclic ether group in the total solid content of the photosensitive composition is preferably 0.1% to 20% by mass. The lower limit is, for example, more preferably 0.5% by mass or more and still more preferably 1% by mass or more. The upper limit is, for example, more preferably 15% by mass or less and still more preferably 10% by mass or less. The compound having a cyclic ether group may be used singly or in combination of two or more kinds thereof. In a case where two or more kinds thereof are used, the total amount thereof is preferably within the above-described range.

<<Silane Coupling Agent>>

The photosensitive composition according to the embodiment of the present invention can contain a silane coupling agent. According to this aspect, adhesiveness of a film to be obtained with a support can be further improved. In the present specification, the silane coupling agent means a silane compound having a hydrolyzable group and other functional groups. In addition, the hydrolyzable group refers to a substituent directly linked to a silicon atom and capable of forming a siloxane bond due to at least one of a hydrolysis reaction or a condensation reaction. Examples of the hydrolyzable group include a halogen atom, an alkoxy group, and an acyloxy group, and an alkoxy group is preferable. That is, it is preferable that the silane coupling agent is a compound having an alkoxysilyl group. Examples of the functional group other than the hydrolyzable group include a vinyl group, a (meth)allyl group, a (meth)acryloyl group, a mercapto group, an epoxy group, an oxetanyl group, an amino group, a ureido group, a sulfide group, an isocyanate group, and a phenyl group, and an amino group, a (meth)acryloyl group, or an epoxy group is preferable. Specific examples of the silane coupling agent include N-β-aminoethyl-γ-aminopropyl methyldimethoxysilane (trade name: KBM-602, manufactured by Shin-Etsu Chemical Co., Ltd.), N-β-aminoethyl-γ-aminopropyl trimethoxysilane (trade name: KBM-603, manufactured by Shin-Etsu Chemical Co., Ltd.), N-β-aminoethyl-γ-aminopropyl triethoxysilane (trade name: KBE-602, manufactured by Shin-Etsu Chemical Co., Ltd.). γ-aminopropyl trimethoxysilane (trade name: KDM-903, manufactured by Shin-Etsu Chemical Co., Ltd.), γ-aminopropyl triethoxysilane (trade name: KBE-903, manufactured by Shin-Etsu Chemical Co., Ltd.), 3-methacryloxypropylmethyl dimethoxysilane (trade name: KBM-502, manufactured by Shin-Etsu Chemical Co., Ltd.), and 3-methacryloxypropyl trimethoxysilane (trade name: KBM-503, manufactured by Shin-Etsu Chemical Co., Ltd.). In addition, specific examples of the silane coupling agent include the compounds described in paragraph Nos. 0018 to 0036 of JP2009-288703A and the compounds described in paragraph Nos. 0056 to 0066 of JP2009-242604A, the contents of which are incorporated herein by reference.

A content of the silane coupling agent in the total solid content of the photosensitive composition is preferably 0.1% to 5% by mass. The upper limit is more preferably 3% by mass or less and still more preferably 2% by mass or less. The lower limit is more preferably 0.5% by mass or more and still more preferably 1% by mass or more. The silane coupling agent may be used singly or in combination of two or more kinds thereof. In a case where two or more kinds thereof are used, the total amount thereof is preferably within the above-described range.

<<Surfactant>>

The photosensitive composition according to the embodiment of the present invention can contain a surfactant. As the surfactant, various surfactants such as a fluorine-based surfactant, a nonionic surfactant, a cationic surfactant, an anionic surfactant, and a silicon-based surfactant can be used. Examples of the surfactant include surfactants described in paragraph Nos. 0238 to 0245 of WO2015/166779A, the contents of which are incorporated herein by reference.

It is preferable that the surfactant is a fluorine-based surfactant. By containing a fluorine-based surfactant in the photosensitive composition, liquid characteristics (particularly, fluidity) are further improved, and liquid saving properties can be further improved. In addition, it is possible to form a film with a small thickness unevenness.

The fluorine content in the fluorine-based surfactant is suitably 3% to 40% by mass, and more preferably 5% to 30% by mass and particularly preferably 7% to 25% by mass. The fluorine-based surfactant in which the fluorine content is within the above-described range is effective in terms of the evenness of the thickness of the coating film or liquid saving properties and the solubility of the surfactant in the photosensitive composition is also good.

Examples of the fluorine-based surfactant include surfactants described in paragraph Nos. 0060 to 0064 of JP2014-041318A (paragraph Nos. 0060 to 0064 of the corresponding WO2014/017669A) and the like, and surfactants described in paragraph Nos. 0117 to 0132 of JP2011-132503A, the contents of which are incorporated herein by reference. Examples of a commercially available product of the fluorine-based surfactant include: MEGAFACE F171, F172, F173, F176, F177, F141, F142, F143, F144, R30, F437, F475, F479, F482, F554, F780, EXP, and MFS-330 (all of which are manufactured by DIC Corporation); FLUORAD FC430, FC431, and FC171 (all of which are manufactured by Sumitomo 3M Ltd.); SURFLON S-382, SC-101, SC-103, SC-104, SC-105, SC-1068, SC-381, SC-383, S-393, and KH-40 (all of which are manufactured by Asahi Glass Co., Ltd.); and POLYFOX PF636, PF656, PF6320, PF6520, and PF7002 (all of which are manufactured by OMNOVA Solutions Inc.).

In addition, as the fluorine-based surfactant, an acrylic compound, which has a molecular structure having a functional group containing a fluorine atom and in which, by applying heat to the molecular structure, the functional group containing a fluorine atom is broken to volatilize a fluorine atom, can also be suitably used. Examples of such a fluorine-based surfactant include MEGAFACE DS series manufactured by DIC Corporation (The Chemical Daily, Feb. 22, 2016; Nikkei Business Daily, Feb. 23, 2016) such as MEGAFACE DS-21.

In addition, it is also preferable that a polymer of a fluorine atom-containing vinyl ether compound having a fluorinated alkyl group or a fluorinated alkylene ether group, and a hydrophilic vinyl ether compound is used as the fluorine-based surfactant. Examples of such a fluorine-based surfactant include fluorine-based surfactants described in JP2016-216602A, the contents of which are incorporated herein by reference.

A block polymer can also be used as the fluorine-based surfactant. As the fluorine-based surfactant, a fluorine-containing polymer compound including a repeating unit derived from a (meth)acrylate compound having a fluorine atom and a repeating unit derived from a (meth)acrylate compound having 2 or more (preferably 5 or more) alkyleneoxy groups (preferably ethyleneoxy groups or propyleneoxy groups) can also be preferably used. In addition, fluorine-containing surfactants described in paragraph Nos. 0016 to 0037 of JP2010-032698A, or the following compounds are also exemplified as the fluorine-based surfactant used in the present invention.

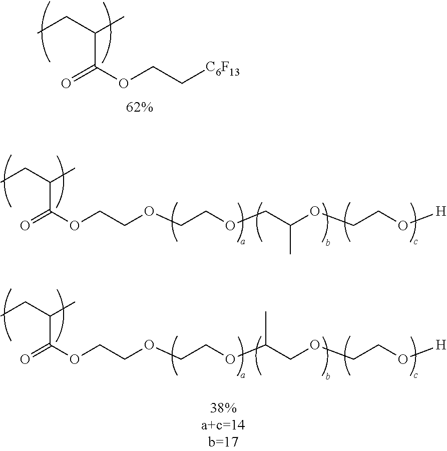

A weight-average molecular weight of the compound is preferably 3000 to 50000 and, for example, 14000. In the compound, “%” representing the proportion of a repeating unit is mol %.

In addition, as the fluorine-based surfactant, a fluorine-containing polymer having an ethylenically unsaturated bond-containing group in the side chain can be used. Specific examples thereof include compounds described in paragraph Nos. 0050 to 0090 and paragraph Nos. 0289 to 0295 of JP2010-164965A, and MEGAFACE RS-101, RS-102, RS-718K, and RS-72-K manufactured by DIC Corporation. In addition, as the fluorine-based surfactant, compounds described in paragraph Nos. 0015 to 0158 of JP2015-117327A can also be used.

A content of the surfactant in the total solid content of the photosensitive composition is preferably 0.001% by mass to 5.0% by mass and more preferably 0.005% to 3.0% by mass. The surfactant may be used singly or in combination of two or more kinds thereof. In a case where two or more kinds thereof are used, the total amount thereof is preferably within the above-described range.

<<Ultraviolet Absorber>>

The photosensitive composition according to the embodiment of the present invention can contain an ultraviolet absorber. As the ultraviolet absorber, a conjugated diene compound, an aminodiene compound, a salicylate compound, a benzophenone compound, a benzotriazole compound, an acrylonitrile compound, a hydroxyphenyltriazine compound, an indole compound, a triazine compound, or the like can be used. Examples of such a compound include compounds described in paragraph Nos. 0038 to 0052 of JP2009-217221A, paragraph Nos. 0052 to 0072 of JP2012-208374A, paragraph Nos. 0317 to 0334 of JP2013-068814A, and paragraph Nos. 0061 to 0080 of JP2016-162946A, the contents of which are incorporated herein by reference. Examples of a commercially available product of the ultraviolet absorber include UV-503 (manufactured by Daito Chemical Co., Ltd). In addition, examples of the benzotriazole compound include MYUA series manufactured by Miyoshi Oil & Fat Co., Ltd. (The Chemical Daily. Feb. 1, 2016). In addition, as the ultraviolet absorber, compounds described in paragraph Nos. 0049 to 0059 of JP6268967B can also be used. A content of the ultraviolet absorber in the total solid content of the photosensitive composition is preferably 0.01% to 10% by mass and more preferably 0.01% to 5% by mass. The ultraviolet absorber may be used singly or in combination of two or more kinds thereof. In a case where two or more kinds thereof are used, the total amount thereof is preferably within the above-described range.

<<Polymerization Inhibitor>>

The photosensitive composition according to the embodiment of the present invention can contain a polymerization inhibitor. Examples of the polymerization inhibitor include hydroquinone, p-methoxyphenol, di-tert-butyl-p-cresol, pyrogallol, tert-butyl catechol, benzoquinone, 4,4′-thiobis(3-methyl-6-tert-butylphenol), 2,2′-methylenebis(4-methyl-6-t-butylphenol), and an N-nitrosophenylhydroxylamine salt (an ammonium salt, a cerous salt, or the like). Among these, p-methoxyphenol is preferable. A content of the polymerization inhibitor in the total solid content of the photosensitive composition is preferably 0.0001% to 5% by mass. The polymerization inhibitor may be used singly or in combination of two or more kinds thereof. In a case of two or more kinds thereof, the total amount thereof is preferably within the above-described range.

<<Solvent>>

The photosensitive composition according to the embodiment of the present invention can contain a solvent. Basically, the organic solvent is not particularly limited as long as it satisfies the solubility of the respective components and the application properties of the photosensitive composition. Examples of the organic solvent include an ester-based solvent, a ketone-based solvent, an alcohol-based solvent, an amide-based solvent, an ether-based solvent, and a hydrocarbon-based solvent. The details of the organic solvent can be found in paragraph No. 0223 of WO2015/166779A, the content of which is incorporated herein by reference. In addition, an ester-based solvent in which a cyclic alkyl group is substituted or a ketone solvent in which a cyclic alkyl group is substituted can also be preferably used. Specific examples of the organic solvent include polyethylene glycol monomethyl ether, dichloromethane, methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, cyclohexyl acetate, cyclopentanone, ethyl carbitol acetate, butyl carbitol acetate, propylene glycol monomethyl ether, propylene glycol monomethyl ether acetate, 3-methoxy-N,N-dimethylpropanamide, and 3-butoxy-N,N-dimethylpropanamide. In this case, it may be preferable that the content of aromatic hydrocarbons (such as benzene, toluene, xylene, and ethylbenzene) as the organic solvent is low (for example, 50 parts per million (ppm) by mass or less, 10 ppm by mass or less, or 1 ppm by mass or less with respect to the total amount of the organic solvent) in consideration of environmental aspects and the like.

A content of the solvent in the photosensitive composition is preferably 10% to 95% by mass. The upper limit is preferably 92.5% by mass or less and more preferably 90% by mass or less. From the viewpoint of application properties, the lower limit is preferably 20% by mass or more, more preferably 50% by mass or more, still more preferably 75% by mass or more, even more preferably 80% by mass or more, and particularly preferably 85% by mass or more.

<<Other Components>>

In the present invention, optionally, the photosensitive composition may further contain a sensitizer, a curing accelerator, a filler, a thermal curing accelerator, a plasticizer, and other auxiliary agents (for example, conductive particles, a filler, an antifoaming agent, a flame retardant, a leveling agent, a peeling accelerator, an aromatic chemical, a surface tension adjuster, or a chain transfer agent). By appropriately containing these components, properties such as film properties can be adjusted. The details of the components can be found in, for example, paragraph No. 0183 of JP2012-003225A (corresponding to paragraph No. 0237 of US2013/0034812A) and paragraph Nos. 0101 to 0104 and 0107 to 0109 of JP2008-250074A, the contents of which are incorporated herein by reference. In addition, as desired, the photosensitive composition according to the embodiment of the present invention may contain a potential antioxidant. Examples of the potential antioxidant include a compound in which a site functioning as an antioxidant is protected by a protective group, and the protective group is eliminated by heating the compound at 100° C. to 250° C. or heating the compound at 80° C. to 200° C. in the presence of an acid or base catalyst so that the compound functions as an antioxidant. Examples of the potential antioxidant include compounds described in WO2014/021023A, WO2017/030005A, and JP2017-008219A. Examples of a commercially available product of the potential antioxidant include ADEKA ARKLS GPA-5001 (manufactured by ADEKA Corporation).

<<Storage Container>>

A storage container for the photosensitive composition is not particularly limited, and a known storage container can be used. In addition, as the storage container, it is also preferable to use a multilayer bottle having an interior wall constituted with six layers from six kinds of resins or a bottle having a 7-layer structure from 6 kinds of resins for the purpose of suppressing infiltration of impurities into raw materials or photosensitive compositions. Examples of such a container include the containers described in JP2015-123351A. In addition, for the purpose of preventing metal elution from the container interior wall, improving storage stability of the photosensitive composition, and suppressing the alteration of components, it is also preferable that the container interior wall is formed of glass, stainless steel, or the like.

<Method for Preparing Photosensitive Composition>

The photosensitive composition according to the embodiment of the present invention can be prepared by mixing the above-described components with each other. In the preparation of the photosensitive composition, all the components may be dissolved and/or dispersed at the same time in a solvent to prepare the photosensitive composition, or the respective components may be appropriately left in two or more solutions or dispersion liquids and mixed to prepare the photosensitive composition upon use (during coating), as desired.

In addition, in the preparation of the photosensitive composition, a process for dispersing the pigment is preferably included. In the process for dispersing the pigment, examples of a mechanical force which is used for dispersing the pigment include compression, pressing, impact, shear, and cavitation. Specific examples of these processes include a beads mill, a sand mill, a roll mill, a ball mill, a paint shaker, a microfluidizer, a high-speed impeller, a sand grinder, a flow jet mixer, high-pressure wet atomization, and ultrasonic dispersion. In addition, in the pulverization of the pigment in a sand mill (beads mill), it is preferable to perform a treatment under the condition for increasing a pulverization efficiency by using beads having small diameters; increasing the filling rate of the beads; or the like. Incidentally, it is preferable to remove coarse particles by filtration, centrifugation, or the like after the pulverization treatment. In addition, as the process and the dispersing machine for dispersing the pigment, the process and the dispersing machine described in “Dispersion Technology Comprehension, published by Johokiko Co., Ltd., Jul. 15, 2005”, “Actual comprehensive data collection on dispersion technology and industrial application centered on suspension (solid/liquid dispersion system), published by Publication Department, Management Development Center, Oct. 10, 1978”, and paragraph No. 0022 of JP2015-157893A can be suitably used. In addition, in the process for dispersing the pigment, a refining treatment of particles in a salt milling step may be performed. With regard to the materials, equipment, treatment conditions, and the like used in the salt milling step, reference can be made to, for example, the description in JP2015-194521A and JP2012-046629A.

It is preferable that, in the preparation of the photosensitive composition, the photosensitive composition is filtered through a filter for the purpose of removing foreign matters, reducing defects, or the like. As the filter, any filters that have been used in the related art for filtration use and the like may be used without particular limitation. Examples of a material of the filter include: a fluororesin such as polytetrafluoroethylene (PTFE); a polyamide resin such as nylon (for example, nylon-6 or nylon-6,6); and a polyolefin resin (including a polyolefin resin having a high density and an ultrahigh molecular weight) such as polyethylene or polypropylene (PP). Among these materials, polypropylene (including a high-density polypropylene) and nylon are preferable.

The pore size of the filter is preferably 0.01 to 7.0 μm, more preferably 0.01 to 3.0 μm, and still more preferably 0.05 to 0.5 μm. In a case where the pore size of the filter is within the above-described range, fine foreign matters can be reliably removed. With regard to the pore size value of the filter, reference can be made to a nominal value of filter manufacturers. As the filter, various filters provided by Nihon Pall Corporation (DFA4201NIEY and the like), Toyo Roshi Kaisha, Ltd., Nihon Entegris K.K. (formerly Nippon Microlith Co., Ltd.), Kitz Micro Filter Corporation, and the like can be used.

In addition, it is preferable that a fibrous filter material is used as the filter. Examples of the fibrous filter material include a polypropylene fiber, a nylon fiber, and a glass fiber. Examples of a commercially available product include SBP type series (SBP008 and the like), TPR type series (TPR002, TPR005, and the like), or SHPX type series (SHPX003 and the like), all manufactured by Roki Techno Co., Ltd.

In a case of using a filter, different filters (for example, a first filter, a second filter, and the like) may be combined. In this case, the filtration with each of the filters may be performed once or may be performed twice or more times. In addition, filters having different pore sizes within the above-described range may be combined. In addition, the filtration through the first filter may be performed with only a dispersion liquid, the other components may be mixed therewith, and then the filtration through the second filter may be performed.

<Film>

A film according to an embodiment of the present invention is a film obtained from the above-described photosensitive composition according to the embodiment of the present invention. A thickness of the film according to the embodiment of the present invention can be adjusted according to the purpose. For example, the film thickness is preferably 20 μm or less, more preferably 10 μm or less, and still more preferably 5 μm or less. The lower limit of the film thickness is preferably 0.1 μm or more, more preferably 0.2 μm or more, and still more preferably 0.3 μm or more.

The film according to the embodiment of the present invention can be used for a color filter, a near infrared transmitting filter, a near infrared cut filter, a black matrix, a light-shielding film, and the like. The film according to the embodiment of the present invention can be preferably used as a colored pixel of a color filter. Examples of the colored pixel include a red pixel, a green pixel, a blue pixel, a magenta pixel, a cyan pixel, and a yellow pixel, and a magenta pixel or a cyan pixel is preferable.

<Method for Producing Film>

The film according to the embodiment of the present invention can be manufactured through a step of forming a photosensitive composition layer on a support with the photosensitive composition according to the embodiment of the present invention, a step of exposing the photosensitive composition layer in a patterned manner, and a step of removing a non-exposed portion of the photosensitive composition layer by development to form a pattern (pixel). A step (pre-baking step) of baking the photosensitive composition layer and a step (post-baking step) of baking the developed pattern (pixel) may be provided, as desired.

In the step of forming a photosensitive composition layer, the photosensitive composition layer is formed on a support using the photosensitive composition. The support is not particularly limited, and can be appropriately selected depending on applications. Examples thereof include a glass substrate and a silicon substrate, and a silicon substrate is preferable. In addition, a charge coupled device (CCD), a complementary metal-oxide semiconductor (CMOS), a transparent conductive film, or the like may be formed on the silicon substrate. In some cases, a black matrix for isolating each pixel is formed on the silicon substrate. In addition, a base layer may be provided on the silicon substrate so as to improve adhesiveness to an upper layer, prevent the diffusion of materials, or planarize the surface of the substrate. A surface contact angle of the base layer is preferably 20° to 70° in a case of being measured with diiodomethane. In addition, the surface contact angle of the base layer is preferably 30° to 80° in a case of being measured with water. In a case where the surface contact angle of the base layer is within the above-described range, coating property of the resin composition is good. The surface contact angle of the base layer can be adjusted by, for example, adding a surfactant.

As a method of applying the photosensitive composition, a known method can be used. Examples thereof include a dropping method (drop casting); a slit coating method; a spray method; a roll coating method; a spin coating method (spin coating); a cast coating method; a slit and spin method; a pre-wet method (for example, a method described in JP2009-145395A), various printing methods such as an ink jet (for example, on-demand type, piezo type, thermal type), a discharge printing such as nozzle jet, a flexo printing, a screen printing, a gravure printing, a reverse offset printing, and a metal mask printing; a transfer method using molds and the like; and a nanoimprinting method. A method for applying the ink jet is not particularly limited, and examples thereof include a method described in “Extension of Use of Ink Jet—Infinite Possibilities in Patent—” (February, 2005, S. B. Research Co., Ltd.) (particularly pp. 115 to 133) and methods described in JP2003-262716A, JP2003-185831A, JP2003-261827A, JP2012-126830A, and JP2006-169325A. In addition, with regard to the method for applying the photosensitive composition, reference can be made to the description in WO2017/030174A and WO2017/018419A, the contents of which are incorporated herein by reference.

The photosensitive composition layer formed on the support may be dried (pre-baked). In a case of producing a film by a low-temperature process, pre-baking may not be performed. In a case of performing the pre-baking, the pre-baking temperature is preferably 150° C. or lower, more preferably 120° C. or lower, and still more preferably 110° C. or lower. The lower limit may be set to, for example, 50° C. or higher, or to 80° C. or higher. The pre-baking time is preferably 10 to 300 seconds, more preferably 40 to 250 seconds, and still more preferably 80 to 220 seconds. The pre-baking can be performed using a hot plate, an oven, or the like.

Next, the photosensitive composition layer is exposed in a patterned manner (exposing step). For example, the photosensitive composition layer can be exposed in a patterned manner using a stepper exposure device or a scanner exposure device through a mask having a predetermined mask pattern. Thus, the exposed portion can be cured.

Examples of the radiation (light) which can be used during the exposure include g-rays and i-rays. In addition, light (preferably light having a wavelength of 180 to 300 nm) having a wavelength of 300 nm or less can be used. Examples of the light having a wavelength of 300 nm or less include KrF-rays (wavelength: 248 nm) and ArF-rays (wavelength: 193 nm), and KrF-rays (wavelength: 248 nm) are preferable. In addition, a long-wave light source of 300 nm or more can be used.

In addition, in a case of exposure, the photosensitive composition layer may be irradiated with light continuously to expose the photosensitive composition layer, or the photosensitive composition layer may be irradiated with light in a pulse to expose the photosensitive composition layer (pulse exposure). The pulse exposure refers to an exposing method in which light irradiation and resting are repeatedly performed in a short cycle (for example, millisecond-level or less).

The irradiation amount (exposure amount) is, for example, preferably 0.03 to 2.5 J/cm² and more preferably 0.05 to 1.0 J/cm². The oxygen concentration during the exposure can be appropriately selected, and the exposure may also be performed, for example, in a low-oxygen atmosphere having an oxygen concentration of 19% by volume or less (for example, 15% by volume, 5% by volume, and substantially oxygen-free) or in a high-oxygen atmosphere having an oxygen concentration of more than 21% by volume (for example, 22% by volume, 30% by volume, and 50% by volume), in addition to an atmospheric air. In addition, the exposure illuminance can be appropriately set, and can be usually selected from a range of 1000 W/m² to 100000 W/m² (for example, 5000 W/m², 15000 W/m², or 35000 W/m²). Appropriate conditions of each of the oxygen concentration and the exposure illuminance may be combined, and for example, a combination of the oxygen concentration of 10% by volume and the illuminance of 1000) W/m², a combination of the oxygen concentration of 35% by volume and the illuminance of 20000 W/m², or the like is available.

Next, a non-exposed portion of the photosensitive composition layer is removed by development to form a pattern (pixel). The removal of the non-exposed portion of the photosensitive composition layer by development can be carried out using a developer. Thus, the photosensitive composition layer of the non-exposed portion in the exposing step is eluted into the developer, and as a result, only a photocured portion remains. The temperature of the developer is preferably, for example, 20° C. to 30° C. The development time is preferably 20 to 180 seconds. In addition, in order to improve residue removing properties, a step of removing the developer by shaking off per 60 seconds and supplying a fresh developer may be repeated multiple times.

Examples of the developer include an organic solvent and an alkali developer, and an alkali developer is preferably used. As the alkali developer, an alkaline aqueous solution (alkali developer) in which an alkaline agent is diluted with pure water is preferable. Examples of the alkali agent include organic alkaline compounds such as ammonia, ethylamine, diethylamine, dimethylethanolamine, diglycol amine, diethanolamine, hydroxyamine, ethylenediamine, tetramethylammonium hydroxide, tetraethylammonium hydroxide, tetrapropylammonium hydroxide, tetrabutylammonium hydroxide, ethyltrimethylammonium hydroxide, benzyltrimethylammonium hydroxide, dimethylbis(2-hydroxyethyl)ammonium hydroxide, choline, pyrrole, piperidine, and 1,8-diazabicyclo-[5.4.0]-7-undecene, and inorganic alkaline compounds such as sodium hydroxide, potassium hydroxide, sodium carbonate, sodium hydrogen carbonate, sodium silicate, and sodium metasilicate. In consideration of environmental aspects and safety aspects, the alkali agent is preferably a compound having a high molecular weight. The concentration of the alkali agent in the alkaline aqueous solution is preferably 0.001% to 10% by mass and more preferably 0.01% to 1% by mass. In addition, the developer may further contain a surfactant. From the viewpoint of transportation, storage, and the like, the developer may be first produced as a concentrated solution and then diluted to a concentration required upon the use. The dilution ratio is not particularly limited, and can be set to, for example, a range of 1.5 to 100 times. In addition, it is also preferable to wash (rinse) with pure water after development. In addition, it is preferable that the rinsing is performed by supplying a rinsing liquid to the photosensitive composition layer after development while rotating the support on which the photosensitive composition layer after development is formed. In addition, it is preferable that the rinsing is performed by moving a nozzle discharging the rinsing liquid from a center of the support to a peripheral edge of the support. In this case, in the movement of the nozzle from the center of the support to the peripheral edge of the support, the nozzle may be moved while gradually decreasing the moving speed of the nozzle. By performing rinsing in this manner, in-plane variation of rinsing can be suppressed. In addition, the same effect can be obtained by gradually decreasing the rotating speed of the support while moving the nozzle from the center of the support to the peripheral edge of the support.

After the development, it is preferable to carry out an additional exposure treatment or a heating treatment (post-baking) after carrying out drying. The additional exposure treatment or the post-baking is a curing treatment after development in order to complete curing. The heating temperature in the post-baking is preferably, for example, 100° C. to 240° C. and more preferably 200° C. to 240° C. The film after development is post-baked continuously or batchwise using a heating unit such as a hot plate, a convection oven (hot air circulation dryer), and a high-frequency heater under the above-described conditions. In a case of performing the additional exposure treatment, light used for the exposure is preferably light having a wavelength of 400 nm or less. In addition, the additional exposure treatment may be carried out by the method described in KR 10-2017-0122130A.

<Optical Filter>

An optical filter according to an embodiment of the present invention has the above-described film according to the embodiment of the present invention. Examples of the type of the optical filter include a color filter, a near infrared cut filter, and a near infrared transmitting filter, and a color filter is preferable. The color filter preferably has the film according to the embodiment of the present invention as a colored pixel thereof.

In the optical filter, a protective layer may be provided on the surface of the film according to the embodiment of the present invention. By providing the protective layer, various functions such as oxygen shielding, low reflection, hydrophilicity/hydrophobicity, and shielding of light (ultraviolet rays, near infrared rays, and the like) having a specific wavelength can be imparted. The thickness of the protective layer is preferably 0.01 to 10 μm and more preferably 0.1 to 5 μm. Examples of a method for forming the protective layer include a method of forming the protective layer by applying a resin composition dissolved in an organic solvent, a chemical vapor deposition method, and a method of attaching a molded resin with an adhesive material. Examples of components constituting the protective layer include a (meth)acrylic resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamidoimide resin, a polyolefin resin, a cyclic olefin resin, a polyester resin, a styrene resin, a polyol resin, a polyvinylidene chloride resin, a melamine resin, a urethane resin, an aramid resin, a polyamide resin, an alkyd resin, an epoxy resin, a modified silicone resin, a fluororesin, a polyacrylonitrile resin, a cellulose resin, Si, C, W, Al₂O₃, Mo, SiO₂, and Si₂N₄, and two or more kinds of these components may be contained. For example, in a case of a protective layer for oxygen shielding, it is preferable that the protective layer contains a polyol resin, SiO₂, and Si₂N₄. In addition, in a case of a protective layer for low reflection, it is preferable that the protective layer contains a (meth)acrylic resin and a fluororesin.

In a case of forming the protective layer by applying a resin composition, as a method for applying the resin composition, a known method such as a spin coating method, a casting method, a screen printing method, and an ink jet method can be used. As the organic solvent included in the resin composition, a known organic solvent (for example, propylene glycol I-monomethyl ether 2-acetate, cyclopentanone, ethyl lactate, and the like) can be used. In a case of forming the protective layer by a chemical vapor deposition method, as the chemical vapor deposition method, a known chemical vapor deposition method (thermochemical vapor deposition method, plasma chemical vapor deposition method, and photochemical vapor deposition method) can be used.

The protective layer may contain, as desired, an additive such as organic or inorganic fine particles, an absorber of light (for example, ultraviolet rays, near infrared rays, and the like) having a specific wavelength, a refractive index adjusting agent, an antioxidant, an adhesive agent, and a surfactant. Examples of the organic or inorganic fine particles include polymer fine particles (for example, silicone resin fine particles, polystyrene fine particles, and melamine resin fine particles), titanium oxide, zinc oxide, zirconium oxide, indium oxide, aluminum oxide, titanium nitride, titanium oxynitride, magnesium fluoride, hollow silica, silica, calcium carbonate, and barium sulfate. As the absorber of light having a specific wavelength, a known absorber can be used. The content of these additives can be appropriately adjusted, but is preferably 0.1% to 70% by mass and still more preferably 1% to 60% by mass with respect to the total mass of the protective layer.

In addition, as the protective layer, the protective layers described in paragraph Nos. 0073 to 0092 of JP2017-151176A can also be used.

The optical filter may have a structure in which each pixel is embedded in a space partitioned in, for example, a lattice form by a partition wall.

<Solid-State Imaging Element>

A solid-state imaging element according to an embodiment of the present invention has the film according to the embodiment of the present invention. The configuration of the solid-state imaging element according to the embodiment of the present invention is not particularly limited as long as the solid-state imaging element is configured to include the film according to the embodiment of the present invention and functions as a solid-state imaging element. Examples of the configuration include the following configurations.

The solid-state imaging element is configured to have a plurality of photodiodes constituting a light receiving area of the solid-state imaging element (a charge coupled device (CCD) image sensor, a complementary metal-oxide semiconductor (CMOS) image sensor, or the like), and a transfer electrode formed of polysilicon or the like on a substrate; have a light-shielding film having openings only over the light receiving section of the photodiodes on the photodiodes and the transfer electrodes: have a device-protective film formed of silicon nitride or the like, which is formed to coat the entire surface of the light-shielding film and the light receiving section of the photodiodes, on the light-shielding film; and have a color filter on the device-protective film. Further, the solid-state imaging element may also be configured, for example, such that it has a light collecting unit (for example, a microlens, which is the same hereinafter) on a device-protective film under a color filter (a side closer to the substrate), or has a light collecting unit on a color filter. In addition, the color filter may have a structure in which each colored pixel is embedded in a space partitioned in, for example, a lattice form by a partition wall. The partition wall in this case preferably has a low refractive index for each colored pixel. Examples of an imaging device having such a structure include the devices described in JP2012-227478A. JP2014-179577A, and WO2018/043654A. In addition, as in JP2019-211559A, an ultraviolet absorbing layer may be provided in the structure of the solid-state imaging element to improve light resistance. An imaging device including the solid-state imaging element according to the embodiment of the present invention can also be used as a vehicle camera or a surveillance camera, in addition to a digital camera or electronic apparatus (mobile phones or the like) having an imaging function.

<Image Display Device>

An image display device according to an embodiment of the present invention has the film according to the embodiment of the present invention. Examples of the image display device include a liquid crystal display device or an organic electroluminescent display device. The definitions of image display devices or the details of the respective image display devices are described in, for example, “Electronic Display Device (Akio Sasaki, Kogyo Chosakai Publishing Co., Ltd., published in 1990)”, “Display Device (Suminki Ibuki, Sangyo Tosho Co., Ltd.)”, and the like. In addition, the liquid crystal display device is described in, for example, “Liquid Crystal Display Technology for Next Generation (edited by Tatsuo Uchida, Kogyo Chosakai Publishing Co., Ltd., published in 1994)”. The liquid crystal display device to which the present invention can be applied is not particularly limited, and can be applied to, for example, liquid crystal display devices employing various systems described in the “Liquid Crystal Display Technology for Next Generation”.

### EXAMPLES

Hereinafter, the present invention will be described in more detail with reference to the examples. The materials, the amounts of materials to be used, the proportions, the treatment details, the treatment procedure, or the like shown in the examples below may be modified appropriately as long as the modifications do not depart from the spirit of the present invention. Therefore, the scope of the present invention is not limited to the specific examples shown below.

<Measuring Method of Weight-Average Molecular Weight>

A weight-average molecular weight (Mw) of a resin was calculated by Gel permeation chromatography (GPC) measurement under the following measurement conditions.

Types of columns: columns formed by connection of TOSOH TSKgel Super HZM-H, TOSOH TSKgel Super HZ4000, and TOSOH TSKgel Super HZ2000

Developing solvent: tetrahydrofuran

Column temperature: 40° C.

Flow rate (amount of a sample to be injected): 1.0 μL (sample concentration: 0.1% by mass)

Device name: HLC-8220GPC manufactured by Tosoh Corporation

Detector: differential refractometer (RI detector)

Calibration curve base resin: polystyrene resin

<Measuring Method of C—C Value (Ethylenically Unsaturated Bond-Containing Group Value)>

A C═C value of each resin was measured by the following method.

The C═C value was obtained by extracting a low-molecular-weight component (a) of an ethylenically unsaturated bond-containing group site from the resin by an alkali treatment, measuring the content thereof by a high performance liquid chromatography (HPLC), and calculating the ethylenically unsaturated bond-containing group value (C═C value) from the following expression based on the measured value.

Specifically, 0.1 g of the resin was dissolved in a tetrahydrofuran and methanol-mixed solution (50 mL/15 mL), 10 mL of a 4 mol/L sodium hydroxide aqueous solution was added thereto, and the mixture was reacted at 40° C. for 2 hours. The reaction solution was neutralized with 10.2 mL of a 4 mol/L methanesulfonic acid aqueous solution, the mixed solution to which 5 mL of ion exchange water and 2 mL of methanol were added was transferred to a 100 mL volumetric flask, and then the mixed solution was diluted in the volumetric flask by methanol to prepare a measurement sample solution. Thereafter, the ethylenically unsaturated bond-containing group value was measured under the following conditions. The content of the low-molecular-weight component (a) was calculated from a calibration curve of the low-molecular-weight component (a) prepared separately, and the ethylenically unsaturated bond-containing group value was calculated from the following expression.

(Expression for Calculating Ethylenically Unsaturated Bond-Containing Group Value)

Ethylenically unsaturated bond-containing group value (mmol/g)=(Content (ppm) of low-molecular-weight component (a)/Molecular weight (g/mol) of low-molecular-weight component (a)/(Weighed value (g) of measurement sample solution)×(Concentration of solid contents (%) of measurement sample solution/100)×10)

### —HPLC Measurement Conditions—

Measuring equipment: Agilent-1200 (manufactured by Agilent Technologies, Inc.)

Column: Synergi 4u Polar-RP 80A manufactured by Phenomenex; 250 mm×4.60 mm (inner diameter)+guard column

Column temperature: 40° C.

Analysis time: 15 minutes

Flow rate: 1.0 mL/min (maximum liquid feeding pressure: 182 bar (18.2 MPa))

Injection amount: 5 μl

Detection wavelength: 210 nm

Eluent: tetrahydrofuran (for stabilizer-free HPLC)/buffer solution (ion exchange aqueous solution containing 0.2 volume % of phosphoric acid and 0.2 volume % of triethylamine)=55/45 (volume %)

In the present specification, % by volume is a value at 25° C.

<Measuring Method of Acid Value>

An acid value of each sample was determined by a neutralization titration using a sodium hydroxide aqueous solution. Specifically, each sample was dissolved in a solvent, the solution was titrated with a sodium hydroxide aqueous solution using a potential difference measurement method to calculate the number of millimoles of the acid included in 1 g of the solid sample, and then the acid value was determined by multiplying the calculated value by 56.1 as a molecular weight of potassium hydroxide (KOH).

<Measuring Method of Amine Value>

Approximately 0.5 g of the sample is precisely weighed and dissolved in 50 mL of acetic acid, and the mixture is titrated with a 0.1 mol/L acetic acid perchlorate solution by an electric titration method (potential difference titration) using an automatic potentiometric titrator (AT-710M: manufactured by KYOTO ELECTRONICS MANUFACTURING CO., LTD.). In addition, a blank test was performed in the same manner as described above to make corrections.

Amine value=a×5.611/c

a: consumption amount (mL) of0.1 mol/L perchloric acid

c: amount (g) of sample

<Production of Dispersion Liquid>

(Dispersion Liquids 1-1 to 1-47)

A mixed solution of a total of 12 parts by mass of a coloring material and a pigment derivative, 3 parts by mass of a dispersant, and 85 parts by mass of propylene glycol monomethyl ether acetate (PGMEA) was mixed and dispersed for 3 hours using a beads mill (zirconia beads, 0.1 mm diameter) to prepare a dispersion liquid. Next, using a high-pressure disperser NANO-3000-10 (manufactured by Nippon BEE Chemical Co., Ltd.) equipped with a pressure reducing mechanism, the dispersion liquid was dispersed under a pressure of 2000 kg/cm³ at a flow rate of 500 g/min. This dispersion treatment was repeated up to 10 times, thereby obtaining dispersion liquids 1-1 to 1-47. Materials shown in the tables below were used as the coloring material, the pigment derivative, and the dispersant.

(Dispersion Liquid 2-1)

A mixed solution of a total of 12 parts by mass of a coloring material and a pigment derivative, 1.8 parts by mass of a dispersant, and 86.2 parts by mass of propylene glycol monomethyl ether acetate (PGMEA) was mixed and dispersed for 3 hours using a beads mill (zirconia beads, 0.1 mm diameter) to prepare a dispersion liquid. Next, using a high-pressure disperser NANO-3000-10 (manufactured by Nippon BEE Chemical Co., Ltd.) equipped with a pressure reducing mechanism, the dispersion liquid was dispersed under a pressure of 2000 kg/cm³ at a flow rate of 500 g/min. This dispersion treatment was repeated up to 10 times, thereby obtaining a dispersion liquid 2-1. Materials shown in the tables below were used as the coloring material, the pigment derivative, and the dispersant.

TABLE 2

| | | | | | Pigment derivative | | |
|---|---|---|---|---|---|---|---|
| | Coloring material | | | | | Blending amount | |
| | Type of | | | | | (part by mass with | |
| | coloring material | | Mixing ratio | | | respect to 100 parts by | |
| | Coloring | Coloring | Coloring | Coloring | | mass of coloring | Dispersant |
| | material 1 | material 2 | material 1 | material 2 | Type | material) | Type |
| Dispersion liquid 1-1 | PG58 | PY185 | 80% | 20% | Pigment derivative 1 | 10 | Dispersant 1 |
| Dispersion liquid 1-2 | PG58 | PY185 | 80% | 20% | Pigment derivative 2 | 10 | Dispersant 1 |
| Dispersion liquid 1-3 | PG58 | PY185 | 80% | 20% | Pigment derivative 3 | 10 | Dispersant 1 |
| Dispersion liquid 1-4 | PG58 | PY185 | 80% | 20% | Pigment derivative 4 | 10 | Dispersant 1 |
| Dispersion liquid 1-5 | PG58 | PY185 | 80% | 20% | Pigment derivative 5 | 10 | Dispersant 1 |
| Dispersion liquid 1-6 | PG58 | PY185 | 80% | 20% | Pigment derivative 6 | 10 | Dispersant 1 |
| Dispersion liquid 1-7 | PG58 | PY185 | 80% | 20% | Pigment derivative 7 | 10 | Dispersant 1 |
| Dispersion liquid 1-8 | PG58 | PY185 | 80% | 20% | Pigment derivative 8 | 10 | Dispersant 1 |
| Dispersion liquid 1-9 | PG58 | PY185 | 80% | 20% | Pigment derivative 9 | 10 | Dispersant 1 |
| Dispersion liquid 1-10 | PG58 | PY185 | 80% | 20% | Pigment derivative 10 | 10 | Dispersant 1 |
| Dispersion liquid 1-11 | PG58 | PYI 85 | 80% | 20% | Pigment derivative 11 | 10 | Dispersant 1 |
| Dispersion liquid 1-12 | PG58 | PY185 | 80% | 20% | Pigment derivative 12 | 10 | Dispersant 1 |
| Dispersion liquid 1-11 | PG58 | PY185 | 80% | 20% | Pigment derivative 13 | 10 | Dispersant 1 |
| Dispersion liquid 1-14 | PG58 | PYI 85 | 80% | 20% | Pigment derivative 13 | 3 | Dispersant 1 |
| Dispersion liquid 1-15 | PG58 | PY185 | 80% | 20% | Pigment derivative 13 | 5 | Dispersant 1 |
| Dispersion liquid 1-16 | PG58 | PY185 | 80% | 20% | Pigment derivative 13 | 20 | Dispersant 1 |
| Dispersion liquid 1-17 | PG58 | PY185 | 80% | 20% | Pigment derivative 13 | 10 | Dispersant 1 |
| Dispersion liquid 1-18 | PG58 | PY185 | 80% | 20% | Pigment derivative 11 | 25 | Dispersant 1 |
| | | | | | Pigment derivative 6 | 5 | |
| Dispersion liquid 1-19 | PG58 | PY185 | 80% | 20% | Pigment derivative 11 | 15 | Dispersant 1 |
| | | | | | Pigment derivative 6 | 15 | |
| Dispersion liquid 1-20 | PG58 | PY185 | 80% | 20% | Pigment derivative 11 | 5 | Dispersant 1 |
| | | | | | Pigment derivative 6 | 25 | |
| Dispersion liquid 1-21 | PG58 | PY185 | 80% | 20% | Pigment derivative 13 | 25 | Dispersant 1 |
| | | | | | Pigment derivative 6 | 5 | |
| Dispersion liquid 1-22 | PG58 | PY185 | 80% | 20% | Pigment derivative 13 | 15 | Dispersant 1 |
| | | | | | Pigment derivative 6 | 15 | |
| Dispersion liquid 1-23 | PG58 | PY185 | 80% | 20% | Pigment derivative 13 | 5 | Dispersant 1 |
| | | | | | Pigment derivative 6 | 25 | |
| Dispersion liquid 1-24 | PG58 | PY185 | 80% | 20% | Pigment derivative 11 | 25 | Dispersant 1 |
| | | | | | Pigment derivative 106 | 5 | |
| Dispersion liquid 1-25 | PG58 | PY185 | 80% | 20% | Pigment derivative 11 | 15 | Dispersant 1 |
| | | | | | Pigment derivative 106 | 15 | |
| Dispersion liquid 1-26 | PG58 | PY185 | 80% | 20% | Pigment derivative 13 | 25 | Dispersant 1 |
| | | | | | Pigment derivative 106 | 5 | |
| Dispersion liquid 1-27 | PG58 | PY185 | 80% | 20% | Pigment derivative 13 | 15 | Dispersant 1 |
| | | | | | Pigment derivative 106 | 15 | |
| Dispersion liquid 1-28 | PG58 | PY185 | 80% | 20.,, | Pigment derivative 12 | 25 | Dispersant 1 |
| | | | | | Pigment derivative 13 | 5 | |
| Dispersion liquid 1-29 | PG58 | PY185 | 80% | 20% | Pigment derisative 12 | 15 | Dispersant 1 |
| | | | | | Pigment derivative 13 | 15 | |
| Dispersion liquid 1-30 | PG58 | PY185 | 80% | 20% | Pigment derivative 12 | 5 | Dispersant 1 |
| | | | | | Pigment derivative 13 | 25 | |

TABLE 3

| | | | | | Pigment derivative | | |
|---|---|---|---|---|---|---|---|
| | | | | | | Blending | |
| | | | | | | amount | |
| | | | | | | (part by | |
| | Coloring material | | | | | mass with | |
| | Type of coloring | | | | | respect to | |
| | material | | Mixing ratio | | | 100 parts by | |
| | Coloring | Coloring | Coloring | Coloring | | mass of | |
| | material | material | material | material | | coloring | Dispersant |
| | 1 | 2 | 1 | 2 | Type | material) | Type |
| Dispersion | PG58 | PY185 | 80% | 20% | Pigment | 10 | Dispersant 2 |
| liquid 1-31 | | | | | derivative 1 | | |
| Dispersion | PG58 | PY185 | 80% | 20% | Pigment | 10 | Dispersant 3 |
| liquid 1-32 | | | | | derivative 1 | | |
| Dispersion | PG58 | PY185 | 80% | 20% | Pigment | It) | Dispersant 4 |
| liquid 1-33 | | | | | derivative 1 | | |
| Dispersion | PG58 | PY185 | 80% | 20% | Pigment | 10 | Dispersant 5 |
| liquid 1-34 | | | | | derivative 1 | | |
| Dispersion | PG58 | PY185 | 80% | 20% | Pigment | 10 | Dispersant 6 |
| liquid 1-35 | | | | | derivative 1 | | |
| Dispersion | PG58 | PY185 | 80% | 20% | Pigment | 10 | Dispersant 7 |
| liquid 1-36 | | | | | derivative I | | |
| Dispersion | PG58 | PY185 | 80% | 20% | Pigment | 10 | Dispersant 8 |
| liquid 1-37 | | | | | derivative 1 | | |
| Dispersion | PG58 | PY185 | 80% | 20% | Pigment | 10 | Dispersant 9 |
| liquid 1-38 | | | | | derivative 1 | | |
| Dispersion | PG58 | PY185 | 80% | 20% | Pigment | 10 | Dispersant 10 |
| liquid 1-39 | | | | | derivative 1 | | |
| Dispersion | PG58 | PY185 | 80% | 20% | Pigment | 10 | Dispersant 11 |
| liquid 1-40 | | | | | derivative 1 | | |
| Dispersion | PG58 | PY185 | 80% | 20% | Pigment | 10 | Dispersant 12 |
| liquid 1-41 | | | | | derivative 1 | | |
| Dispersion | PG58 | PY185 | 80% | 20% | Pigment | 10 | Dispersant 13 |
| liquid 1-42 | | | | | derivative 1 | | |
| Dispersion | PG58 | PY185 | 80% | 20% | Pigment | 10 | Dispersant 14 |
| liquid 1-43 | | | | | derivative 1 | | |
| Dispersion | PG58 | PY185 | 80% | 20% | Pigment | 10 | Dispersant |
| liquid 1-44 | | | | | derivative 1 | | 1/dispersant |
| | | | | | | | 2-7/3 |
| | | | | | | | (mass ratio) |
| Dispersion | PG58 | PY185 | 80% | 20% | Pigment | 10 | Dispersant |
| liquid 1-45 | | | | | derivative 1 | | 1/dispersant |
| | | | | | | | 2-3/7 |
| | | | | | | | (mass ratio) |
| Dispersion | PG58 | PY185 | 80% | 20% | Pigment | 10 | Dispersant 1 |
| liquid 1-46 | | | | | derivative 14 | | |
| Dispersion | PG58 | PY185 | 80% | 20% | Pigment | 10 | Dispersant 1 |
| liquid 1-47 | | | | | derivative 15 | | |
| Dispersion | PG58 | PY185 | 80% | 20% | Pigment | 10 | Dispersant 1 |
| liquid 2-1 | | | | | derivative 1 | | |

(Dispersion Liquids 3-1 to 3-116)

Dispersion liquids 3-1 to 3-116 were produced in the same manner as in the dispersion liquid 1-1, except that the type of the pigment was changed to the type shown in the tables below.

TABLE 4

| | Type of coloring material | | | | Mixing ratio | | | |
|---|---|---|---|---|---|---|---|---|
| | Coloring | Coloring | Coloring | Coloring | Coloring | Coloring | Coloring | Coloring |
| | material | material | material | material | material | material | material | material |
| | 1 | 2 | 3 | 4 | 1 | 2 | 3 | 4 |
| Dispersion liquid 3-1 | PR254 | PY139 | — | — | 69.0% | 31.0% | — | — |
| Dispersion liquid 3-2 | PR272 | PY139 | — | — | 62.3% | 37.7% | — | — |
| Dispersion liquid 3-3 | PR272 | PY254 | PY139 | — | 43.8% | 37.5% | 18.8% | — |
| Dispersion liquid 3-4 | PR272 | PY254 | PY139 | — | 34.7% | 30.4% | 35.0% | — |
| Dispersion liquid 3-5 | PR254 | PO71 | PY139 | — | 39.2% | 49.1% | 11.8% | — |
| Dispersion liquid 3-6 | PR254 | PV19 | PY139 | — | 62.0% | 10.0% | 28.0% | — |
| Dispersion liquid 3-7 | PR264 | PV19 | PY139 | — | 62.0% | 10.0% | 28.0% | — |
| Dispersion liquid 3-8 | PR264 | PY139 | — | — | 80.0% | 20.0% | — | — |
| Dispersion liquid 3-9 | PR269 | PY139 | — | — | 80.0% | 20.0% | — | — |
| Dispersion liquid 3-10 | PR291 | PY139 | — | — | 80.0% | 20.0% | — | — |
| Dispersion liquid 3-11 | PR296 | PY139 | — | — | 80.0% | 20.0% | — | — |
| Dispersion liquid 3-12 | PR297 | PY139 | — | — | 80.0% | 20.0% | — | — |
| Dispersion liquid 3-13 | PR272 | PR264 | PY139 | — | 34.7% | 30.4% | 35.0% | — |
| Dispersion liquid 3-14 | PR272 | PR269 | PY139 | — | 34.7% | 30.4% | 35.0% | — |
| Dispersion liquid 3-15 | PR272 | PR291 | PY139 | — | 34.7% | 30.4% | 35.0% | — |
| Dispersion liquid 3-16 | PR272 | PR296 | PY139 | — | 34.7% | 30.4% | 35.0% | — |
| Dispersion liquid 3-17 | PR272 | PR297 | PY139 | — | 34.7% | 30.4% | 35.0% | — |
| Dispersion liquid 3-18 | PR264 | PR254 | PY139 | — | 34.7% | 304% | 35.0% | — |
| Dispersion liquid 3-19 | PR269 | PR254 | PY139 | — | 34.7% | 30.4% | 35.0% | — |
| Dispersion liquid 3-20 | PR291 | PR254 | PY139 | — | 34.7% | 30.4% | 35.0% | — |
| Dispersion liquid 3-21 | PR296 | PR254 | PY139 | — | 34.7% | 30.4% | 35.0% | — |
| Dispersion liquid 3-22 | PR297 | PR251 | PY139 | — | 31.7% | 30.4% | 35.0% | — |
| Dispersion liquid 3-23 | PG36 | PY185 | — | — | 80.0% | 20.0% | — | — |
| Dispersion liquid 3-24 | PG36 | PY150 | — | — | 55.0% | 45.0% | — | — |
| Dispersion liquid 3-25 | PG36 | PY150 | PY185 | — | 73.8% | 19.4% | 6.7% | — |
| Dispersion liquid 3-26 | PG36 | PY150 | PY129 | — | 60.0% | 20.0% | 20.0% | — |
| Dispersion liquid 3-27 | PG36 | PY215 | — | — | 55.0% | 45.0% | — | — |
| Dispersion liquid 3-28 | PG36 | PY215 | PY185 | — | 73.8% | 19.4% | 6.7% | — |
| Dispersion liquid 3-29 | PG36 | PY215 | PY129 | — | 60.0% | 20.0% | 20.0% | — |
| Dispersion liquid 3-30 | PG36 | PY215 | PY129 | PY185 | 65.0% | 15.0% | 15.0% | 5.0% |
| Dispersion liquid 3-31 | PG36 | PY231 | — | — | 80.0% | 20.0% | — | — |
| Dispersion liquid 3-32 | PG36 | PY150 | PY231 | — | 73.8% | 19.4% | 6.7% | — |
| Dispersion liquid 3-33 | PG36 | PY215 | PY231 | — | 73.8% | 19.4% | 6.7% | — |
| Dispersion liquid 3-34 | PG36 | PY233 | — | — | 80.0% | 20.0% | — | — |
| Dispersion liquid 3-35 | PG36 | PY150 | PY233 | — | 73.8% | 19.4% | 6.7% | — |
| Dispersion liquid 3-36 | PG36 | PY215 | PY233 | — | 73.8% | 19.4% | 6.7% | — |
| Dispersion liquid 3-37 | PG58 | PY185 | — | — | 80.0% | 20.0% | — | — |
| Dispersion liquid 3-38 | PG58 | PY150 | — | — | 55.0% | 45.0% | — | — |
| Dispersion liquid 3-39 | PG58 | PY150 | PY185 | — | 73.8% | 19.4% | 6.7% | — |
| Dispersion liquid 3-40 | PG58 | PY150 | PY129 | — | 60.0% | 20.0% | 20.0% | — |

TABLE 5

| | Type of coloring material | | | | Mixing ratio | | | |
|---|---|---|---|---|---|---|---|---|
| | Coloring | Coloring | Coloring | Coloring | Coloring | Coloring | Coloring | Coloring |
| | material | material | material | material | material | material | material | material |
| | 1 | 2 | 3 | 4 | 1 | 2 | 3 | 4 |
| Dispersion liquid | PG58 | PY215 | — | — | 55.0% | 45.0% | — | — |
| 3-41 | | | | | | | | |
| Dispersion liquid | PG58 | PY215 | PY185 | — | 73.8% | 19.4% | 6.7% | — |
| 3-42 | | | | | | | | |
| Dispersion liquid | PG58 | PY215 | PY129 | — | 60.0% | 20.0% | 20.0% | — |
| 343 | | | | | | | | |
| Dispersion liquid | PG58 | PY215 | PY129 | PY185 | 65.0% | 15.0% | 15.0% | 5.0% |
| 344 | | | | | | | | |
| Dispersion liquid | PG58 | PY231 | — | — | 80.0% | 20.0% | — | — |
| 3-45 | | | | | | | | |
| Dispersion liquid | PG58 | PY150 | PY231 | — | 73.8% | 19.4% | 6.7% | — |
| 346 | | | | | | | | |
| E)isperstisn liquid | PG58 | PY215 | PY231 | — | 73.8% | 19.4% | 6.7% | — |
| 3-47 | | | | | | | | |
| Ilispersion liquid | PG58 | PY233 | — | — | 80.0% | 20.0% | — | — |
| 3-48 | | | | | | | | |
| Dispersion liquid | PG58 | PY150 | PY233 | — | 73.8% | 19.4% | 6.7% | — |
| 3-49 | | | | | | | | |
| Dispersion liquid | PG58 | PY215 | PY233 | — | 73.8% | 19.4% | 6.7% | — |
| 3-50 | | | | | | | | |
| Dispersion liquid | PG63 | PY185 | — | — | 80.0% | 20.0% | — | — |
| 3-51 | | | | | | | | |
| Dispersion liquid | PG63 | PY150 | — | — | 55.0% | 45.0% | — | — |
| 3-52 | | | | | | | | |
| Dispersion liquid | PG63 | PY150 | PY185 | — | 73.8% | 19.4% | 6.7% | — |
| 3-53 | | | | | | | | |
| Dispersion liquid | PG63 | PY150 | PY129 | — | 60.0% | 20.0% | 20.0% | — |
| 3-54 | | | | | | | | |
| Dispersion liquid | PG63 | PY215 | — | — | 55.0% | 45.0% | — | — |
| 3-55 | | | | | | | | |
| Dispersion liquid | PG63 | PY215 | PY185 | — | 73.8% | 19.4% | 6.7% | — |
| 3-56 | | | | | | | | |
| Dispersion liquid | PG63 | PY215 | PY129 | — | 60.0% | 20.0% | 20.0% | — |
| 3-57 | | | | | | | | |
| Dispersion liquid | PG63 | PY215 | PY129 | PY185 | 65.0% | 15.01% | 15.0% | 5.0% |
| 3-58 | | | | | | | | |
| Dispersion liquid | PG63 | PR231 | — | — | 80.0% | 20.0% | — | — |
| 3-59 | | | | | | | | |
| Dispersion liquid | PG63 | PY150 | PY231 | — | 73.8% | 19.4% | 6.7% | — |
| 3-60 | | | | | | | | |
| Dispersion liquid | PG63 | PY215 | PY231 | — | 73.8% | 19.4% | 6.7% | — |
| 3-61 | | | | | | | | |
| Dispersion liquid | PG63 | PY233 | — | — | 80.0% | 20.0% | — | — |
| 3-62 | | | | | | | | |
| Dispersion liquid | PG63 | PY150 | PY233 | — | 73.8% | 19.4% | 6.7% | — |
| 3-63 | | | | | | | | |
| Dispersion liquid | PG63 | PY215 | PY233 | — | 73.8% | 19.4% | 6.7% | — |
| 3-64 | | | | | | | | |
| Dispersion liquid | PB15:6 | PV23 | — | — | 78.5% | 21.5% | — | — |
| 3-65 | | | | | | | | |
| Dispersion liquid | PB15:6 | PV37 | — | — | 78.5% | 21.5% | — | — |
| 3-66 | | | | | | | | |
| Dispersion liquid | PB15:6 | Xanthene | — | — | 63.5% | 36.5% | — | — |
| 3-67 | | pigment | | | | | | |
| Dispersion liquid | PB15:6 | PV2 | — | — | 63.5% | 36.5% | — | — |
| 3-68 | | | | | | | | |
| Dispersion liquid | PG7 | — | — | — | 100.0% | — | — | — |
| 3-69 | | | | | | | | |
| Dispersion liquid | PG36 | — | — | — | 100.0% | — | — | — |
| 3-70 | | | | | | | | |
| Dispersion liquid | PG58 | — | — | — | 100.0% | — | — | — |
| 3-71 | | | | | | | | |
| Dispersion liquid | PG63 | — | — | — | 100.0% | — | — | — |
| 3-72 | | | | | | | | |
| Dispersion liquid | PG7 | PG36 | — | — | 71.4% | 28.6% | — | — |
| 3-73 | | | | | | | | |
| Dispersion liquid | PG7 | PG36 | PB15:4 | — | 61.6% | 24.7% | 13.6% | — |
| 3-74 | | | | | | | | |
| Dispersion liquid | PG7 | PG36 | PB16 | — | 61.6% | 24.7% | 13.6% | — |
| 3-75 | | | | | | | | |
| Dispel-mon liquid | PB15:3 | — | — | — | 100.0% | — | — | — |
| 3-76 | | | | | | | | |
| Dispersion liquid | PB15:4 | — | — | — | 100.0% | — | — | — |
| 3-77 | | | | | | | | |
| Dispersion liquid | PB15:6 | — | — | — | 100.0% | — | — | — |
| 3-78 | | | | | | | | |
| Dispersion liquid | PB16 | — | — | — | 100.0% | — | — | — |
| 3-79 | | | | | | | | |
| Dispersion liquid | Al | — | — | — | 100.0% | — | — | — |
| 3-80 | phthalocyanine | | | | | | | |

TABLE 6

| | Type of coloring material | | | | Mixing ratio | | | |
|---|---|---|---|---|---|---|---|---|
| | Coloring | Coloring | Coloring | Coloring | Coloring | Coloring | Coloring | Coloring |
| | material | material | material | material | material | material | material | material |
| | 1 | 2 | 3 | 4 | 1 | 2 | 3 | 4 |
| Dispersion liquid | PR122 | — | — | — | 100.0% | — | — | — |
| 3-81 | | | | | | | | |
| Dispersion liquid | PR177 | — | — | — | 100.0% | — | — | — |
| 3-82 | | | | | | | | |
| Dispersion liquid | PR202 | — | — | — | 100.0% | — | — | — |
| 3-83 | | | | | | | | |
| Dispersion liquid | PR209 | — | — | — | 100.0% | — | — | — |
| 3-84 | | | | | | | | |
| Dispersion liquid | PV2 | — | — | — | 100.0% | — | — | — |
| 3-85 | | | | | | | | |
| Dispersion liquid | PV19 | — | — | — | 100.0% | — | — | — |
| 3-86 | | | | | | | | |
| Dispersion liquid | PV23 | — | — | — | 100.0% | — | — | — |
| 3-87 | | | | | | | | |
| Dispersion liquid | PR122 | Xanthene | — | — | 43.1% | 56.9% | — | — |
| 3-88 | | pigment | | | | | | |
| Dispersion liquid | PR177 | Xanthene | — | — | 43.1% | 56.9% | — | — |
| 3-89 | | pigment | | | | | | |
| Dispersion liquid | PR202 | Xanthene | — | — | 43.1% | 56.9% | — | — |
| 3-90 | | pigment | | | | | | |
| Dispersion liquid | PR209 | Xanthene | — | — | 43.1% | 56.9% | — | — |
| 3-91 | | pigment | | | | | | |
| Dispersion liquid | PV2 | Xanthene | — | — | 25.0% | 75.0% | — | — |
| 3-92 | | pigment | | | | | | |
| Dispersion liquid | PV19 | Xanthene | — | — | 25.0% | 75.0% | — | — |
| 3-93 | | pigment | | | | | | |
| Dispersion liquid | PV23 | Xanthene | — | — | 25.0% | 75.0% | — | — |
| 3-94 | | pigment | | | | | | |
| Dispersion liquid | PY150 | — | — | — | 100.0% | — | — | — |
| 3-95 | | | | | | | | |
| Dispersion liquid | PY185 | — | — | — | 100.0% | — | — | — |
| 3-96 | | | | | | | | |
| Dispersion liquid | PY129 | — | — | — | 100.0% | — | — | — |
| 3-97 | | | | | | | | |
| Dispersion liquid | PY139 | — | — | — | 100.0% | — | — | — |
| 3-98 | | | | | | | | |
| Dispersion liquid | PY215 | — | — | — | 100.0% | — | — | — |
| 3-99 | | | | | | | | |
| Dispersion liquid | PY228 | — | — | — | 100.0% | — | — | — |
| 3-100 | | | | | | | | |
| Dispersion liquid | PY211 | — | — | — | 100.0% | — | — | — |
| 3-101 | | | | | | | | |
| Dispersion liquid | PY233 | — | — | — | 100.0% | — | — | — |
| 3-102 | | | | | | | | |
| Dispersion liquid | PR254 | PY139 | PV23 | PB15:6 | 37.8% | 16.2% | 8.1% | 37.8% |
| 3-103 | | | | | | | | |
| Dispersion liquid | PR264 | PY139 | PV23 | PB15:6 | 37.8% | 16.2% | 8.1% | 37.8% |
| 3-104 | | | | | | | | |
| Dispersion liquid | PR269 | PY139 | PV23 | PB15:6 | 37.8% | 16.2% | 8.1% | 37.8% |
| 3-105 | | | | | | | | |
| Dispersion liquid | PR272 | PY130 | PV23 | PB15:6 | 37.8% | 16.2% | 8.1% | 37.8% |
| 3-106 | | | | | | | | |
| Dispersion liquid | PR291 | PY139 | PV23 | PB15:6 | 37.8% | 16.2% | 8.1% | 37.8% |
| 3-107 | | | | | | | | |
| Dispersion liquid | PR296 | PY139 | PV23 | PB15:6 | 37.8% | 16.2% | 8.1% | 37.8% |
| 3-108 | | | | | | | | |
| Dispersion liquid | PR297 | PY139 | PV23 | PB15:6 | 37.8% | 16.2% | 8.1% | 37.8% |
| 3-109 | | | | | | | | |
| Dispersion liquid | PR254 | PY215 | PB16 | — | 50.0% | 16.7% | 33.3% | — |
| 3-110 | | | | | | | | |
| Dispersion liquid | PR264 | PY215 | PB16 | — | 50.0% | 16.7% | 33.3% | — |
| 3-111 | | | | | | | | |
| Dispersion liquid | Perylene | PY139 | PV23 | PB15:6 | 37.8% | 16.2% | 8.1% | 37.8% |
| 3-112 | black | | | | | | | |
| Dispersion liquid | Bisbenzo- | PY139 | PV33 | PB15:6 | 37.8% | 16.2% | 8.1% | 37.8% |
| 3-113 | furanone | | | | | | | |
| Dispersion liquid | IR coloring | — | — | — | 100.0% | — | — | — |
| 3-114 | material 1 | | | | | | | |
| Dispersion liquid | IR coloring | — | — | — | 100.0% | — | — | — |
| 3-115 | material 2 | | | | | | | |
| Dispersion liquid | IR coloring | — | — | — | 100.0% | — | — | — |
| 3-116 | material 3 | | | | | | | |

(Dispersion Liquids r1-1 to r1-10)

A mixed solution of a total of 12 parts by mass of a pigment and a pigment derivative, 3 parts by mass of a dispersant, and 85 pans by mass of propylene glycol monomethyl ether acetate (PGMEA) was mixed and dispersed for 3 hours using a beads mill (zirconia beads, 0.1 mm diameter) to prepare a dispersion liquid. Next, using a high-pressure disperser NANO-3000-10 (manufactured by Nippon BEE Chemical Co., Ltd.) equipped with a pressure reducing mechanism, the dispersion liquid was dispersed under a pressure of 2000 kg/cm³ at a flow rate of 500 g/min. This dispersion treatment was repeated up to 10 times, thereby obtaining dispersion liquids r1-1 to r1-10. Materials shown in the tables below were used as the pigment, the pigment derivative, and the dispersant.

(Dispersion Liquid r2-1)

A mixed solution of a total of 12 parts by mass of a coloring material and a pigment derivative, 1.8 parts by mass of a dispersant, and 86.2 parts by mass of propylene glycol monomethyl ether acetate (PGMEA) was mixed and dispersed for 3 hours using a beads mill (zirconia beads, 0.1 mm diameter) to prepare a dispersion liquid. Next, using a high-pressure disperser NANO-3000-10 (manufactured by Nippon BEE Chemical Co., Ltd.) equipped with a pressure reducing mechanism, the dispersion liquid was dispersed under a pressure of 2000 kg/cm³ at a flow rate of 500 g/min. This dispersion treatment was repeated up to 10 times, thereby obtaining a dispersion liquid r2-1. Materials shown in the tables below were used as the coloring material, the pigment derivative, and the dispersant.

[Table 7]

The details of the materials indicated by the abbreviations in the tables showing the formulation of the dispersion liquids described above are as follows.

(Coloring Material)

PG7: C. I. Pigment Green 7 (green pigment)

PG36: C. I. Pigment Green 36 (green pigment)

PG58: C. I. Pigment Green 58 (green pigment)

PG63: C. I. Pigment Green 63 (green pigment)

PY129: C. I. Pigment Yellow 129 (yellow pigment)

PY139: C. I. Pigment Yellow 139 (yellow pigment)

PY150: C. I. Pigment Yellow 150 (yellow pigment)

PY185: C. I. Pigment Yellow 185 (yellow pigment)

PY215: C. I. Pigment Yellow 215 (yellow pigment)

PY228: C. I. Pigment Yellow 228 (yellow pigment)

PY231: C. I. Pigment Yellow 231 (yellow pigment)

PY233: C. I. Pigment Yellow 233 (yellow pigment)

PR122: C. I. Pigment Red 122 (red pigment)

PR177: C. I. Pigment Red 177 (red pigment)

PR202: C. I. Pigment Red 202 (red pigment)

PR209: C. I. Pigment Red 209 (red pigment)

PR254: C. I. Pigment Red 254 (red pigment)

PR272: C. I. Pigment Red 272 (red pigment)

PR264: C. I. Pigment Red 264 (red pigment)

PR269: C. I. Pigment Red 269 (red pigment)

PR291: C. I. Pigment Red 291 (red pigment)

PR296: C. I. Pigment Red 2% (red pigment)

PR297: C. I. Pigment Red 297 (red pigment)

PO71: C. I. Pigment Orange 71 (orange pigment)

PB15:3: C. I. Pigment Blue 15:3 (blue pigment)

PB15:4: C. I. Pigment Blue 15:4 (blue pigment)

PB15:6: C. I. Pigment Blue 15:6 (blue pigment)

PB16: C. I. Pigment Blue 16 (blue pigment)

PV2: C. I. Pigment Violet 2 (violet pigment)

PV19: C. I. Pigment Violet 19 (violet pigment)

PV23: C. I. Pigment Violet 23 (violet pigment)

PV37: C. I. Pigment Violet 37 (violet pigment)

A1 phthalocyanine: compound having the following structure (blue pigment)

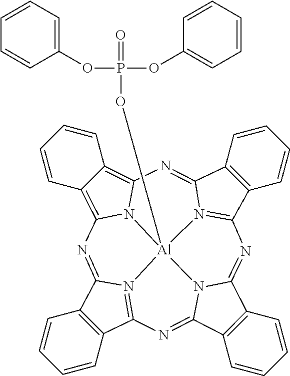

Xanthene dye: compound having the following structure (chromatic dye)

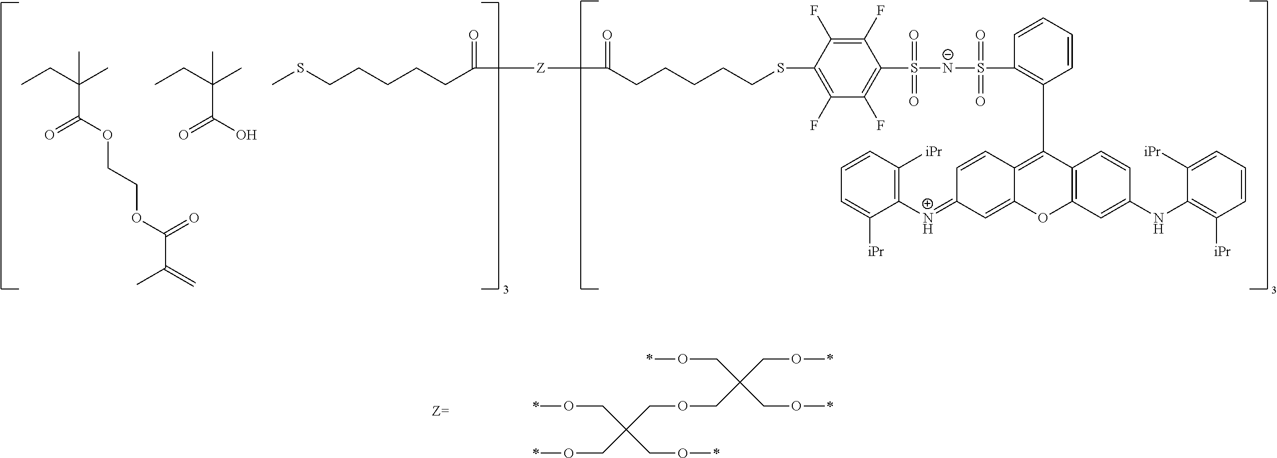

Perylene black: compound having the following structure (black pigment)

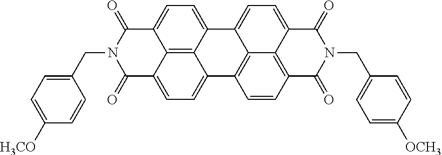 [SMILES: COc1ccc(CN2C(=O)c3ccc4c5ccc6c7c(ccc(c8ccc(c3c48)C2=O)c75)C(=O)N(Cc2ccc(OC)cc2)C6=O)cc1]

Bisbenzofuranone: compound having the following structure (black pigment)

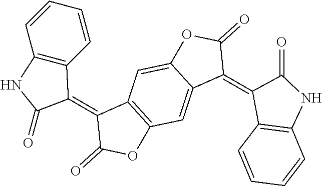 [SMILES: O=C1Nc2ccccc2/C1=C1/C(=O)Oc2cc3c(cc21)OC(=O)/C3=C1\C(=O)Nc2ccccc21]

IR coloring material 1: compound having the following structure (near infrared absorbing pigment)

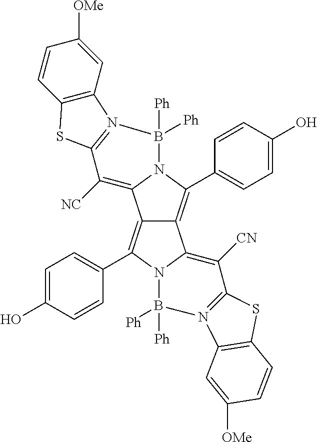

IR coloring material 2: compound having the following structure (near infrared absorbing pigment)

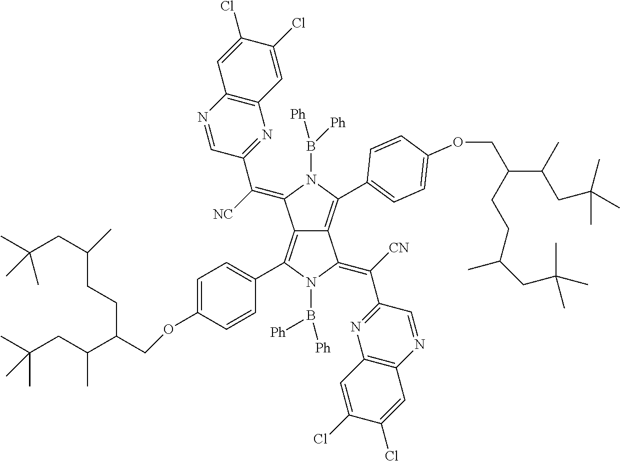 [SMILES: CC(CCC(COc1ccc(-c2c3/c(=C(\C#N)c4cnc5cc(Cl)c(Cl)cc5n4)n(B(c4ccccc4)c4ccccc4)c(-c4ccc(OCC(CCC(C)CC(C)(C)C)C(C)CC(C)(C)C)cc4)c3/c(=C(\C#N)c3cnc4cc(Cl)c(Cl)cc4n3)n2B(c2ccccc2)c2ccccc2)cc1)C(C)CC(C)(C)C)CC(C)(C)C]

IR coloring material 3: compound having the following structure (near infrared absorbing pigment)

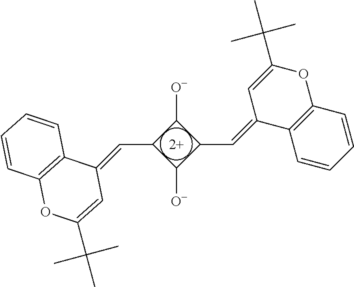

(Pigment Derivative)

With regard to each pigment derivative described below, the maximum value (εmax) of a molar absorption coefficient in a wavelength range of 400 to 7M) nm was measured as follows. 20 mg of each compound was dissolved in 200 mL of methanol, and methanol was added to 2 mL of this solution so as to be 50 mL. The absorbance of this solution was measured in a wavelength range of 200 to 800 nm using Cary 5000 UV-Vis-NIR spectrophotometer (manufactured by Agilent Technologies. Inc.), and the maximum value of this measured value was standardized by molar concentration to calculate εmax.

[Pigment Derivative Having εMax of 3000 L·mol⁻¹·cm⁻¹ or Less]

Pigment derivative 1: compound which was the compound No. C-1 shown in Table 1 as the specific example of the pigment derivative B1 (εmax: 1M L·mol⁻¹·cm⁻¹ or less, basic derivative)

Pigment derivative 2: compound which was the compound No. C-20 shown as the specific example of the pigment derivative B1 (εmax: more than 1000 L·mol⁻¹·cm⁻¹ and 3000 L·mol⁻¹·cm⁻¹ or less, basic derivative)

Pigment derivative 3: compound which was the compound No. C-36 shown as the specific example of the pigment derivative B1 (εmax: 100 L·mol⁻¹·cm⁻¹ or less, basic derivative)

Pigment derivative 4: compound which was the compound No. C-51 shown as a specific example of the pigment derivative B1 (εmax: more than 100 L·mol⁻¹·cm⁻¹ and 1000 L·mol⁻¹·cm⁻¹ or less, basic derivative)

Pigment derivative 5: compound which was the compound No. C-87 shown as the specific example of the pigment derivative B1 (εmax: 100 L·mol⁻¹·cm⁻¹ or less, basic derivative)

Pigment derivative 6: compound having the following structure (εmax: 100 L·mol⁻¹·cm⁻¹ or less, acidic derivative)

Pigment derivative 7: compound having the following structure (εmax: 100 L·mol⁻¹·cm⁻¹ or less, acidic derivative)

Pigment derivative 8: compound having the following structure (εmax: 100 L·mol⁻¹·cm⁻¹ or less, acidic derivative)

Pigment derivative 9: compound having the following structure (εmax: 100 L·mol⁻¹·cm⁻¹ or less, acidic derivative)

Pigment derivative 10: compound having the following structure (εmax: 100 L·mol⁻¹·cm⁻¹ or less, acidic derivative)

Pigment derivative 11: compound which was the compound No. C-60 shown as the specific example of the pigment derivative B1 (εmax: 100 L·mol⁻¹·cm⁻¹ or less, basic derivative)

Pigment derivative 12: coloring agent derivative 1 described in Table 2 of paragraph No. 0296 of JP2019-133154A (εmax: 100 L·mol⁻¹·cm⁻¹ or less, basic derivative)

Pigment derivative 13: coloring agent derivative 2 described in Table 2 of paragraph No. 0296 of JP2019-133154A (max: 100 L·mol⁻¹·cm⁻¹ or less, basic derivative)

Pigment derivative 14: additive 79 described in Table 1 of paragraph No. 0026 of JP2018-150498A (εmax: 100 L·mol⁻¹·cm⁻¹ or less, basic derivative)

Pigment derivative 15: additive 157 described in Table 1 of paragraph No. 0026 of JP2019-150498A (εmax: 100 L·mol⁻¹·cm⁻¹ or less, basic derivative)

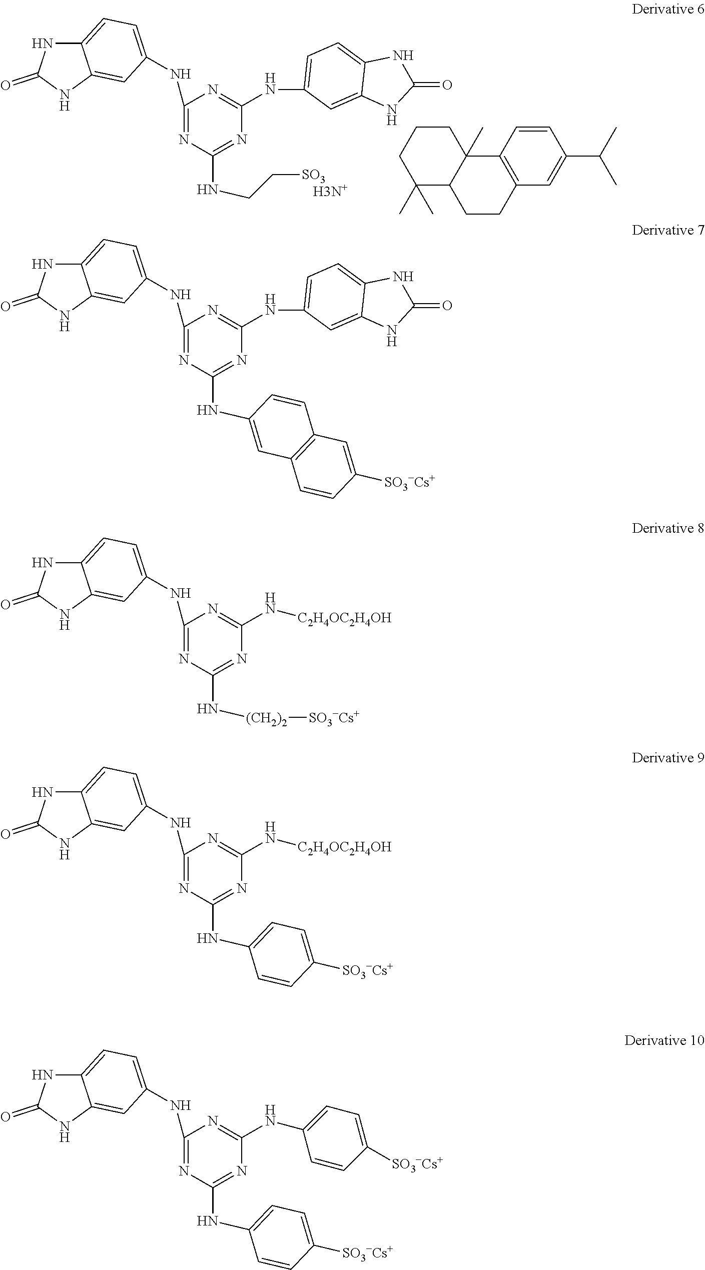

[Pigment Derivative Having εMax of More than 3000 L·mol⁻¹·cm⁻¹]

Derivative 101: compound having the following structure (yellow, basic derivative)

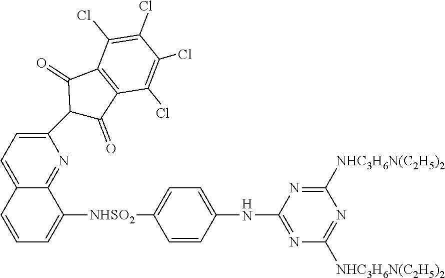

Derivative 102: compound having the following structure (red, basic derivative)

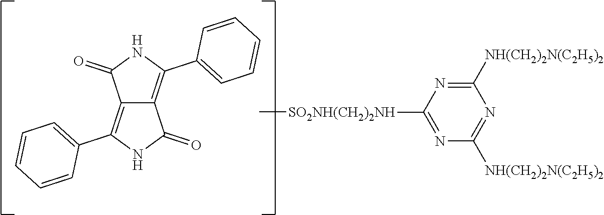

Derivative 103: compound having the following structure (yellow, basic derivative)

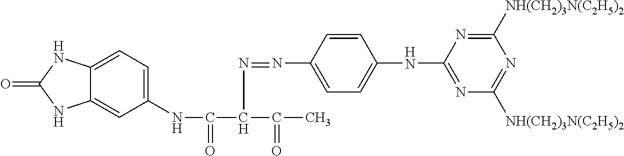

Derivative 104: compound having the following structure (blue, basic derivative)

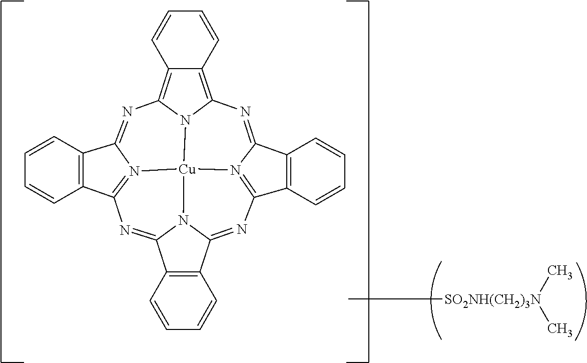

Derivative 105: compound having the following structure (violet, basic derivative)

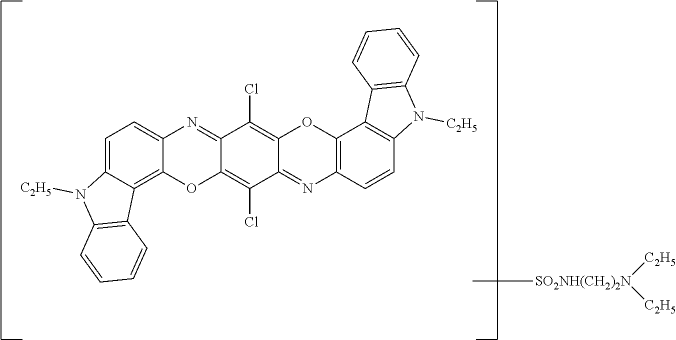

Derivative 106: compound having the following structure (yellow, basic derivative)

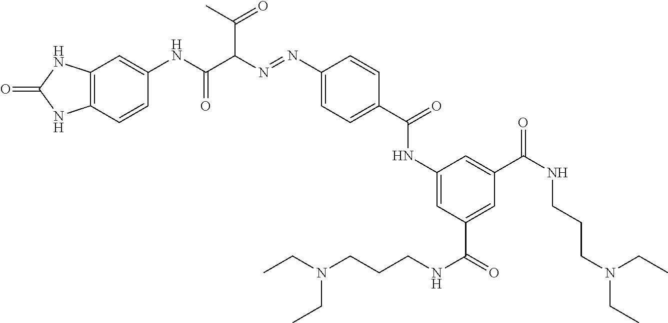 [SMILES: CCN(CC)CCCNC(=O)c1cc(NC(=O)c2ccc(/N=N/C(C(C)=O)C(=O)Nc3ccc4[nH]c(=O)[nH]c4c3)cc2)cc(C(=O)NCCCN(CC)CC)c1]

The derivatives 101 to 106 all exhibited a chromatic hue, and εmax was more than 3000 L·mol⁻¹·cm⁻¹.

(Dispersant)

[Dispersant Having Ethylenically Unsaturated Bond-Containing Group]

Dispersant 1: resin produced according to the method described in paragraph No. 0287 of JP2019-133154A (dispersant having an ethylenically unsaturated bond-containing group: weight-average molecular weight: 13000, C═C value: 0.63 mmol/g)

Dispersant 2: resin produced according to the method described in the resin type dispersant (C2-42) of paragraph Nos. 0287 to 0289 of JP2019-133154A (dispersant having an ethylenically unsaturated bond-containing group; weight-average molecular weight: 13000, C═C value: 1.14 mmol/g)

Dispersant 3: resin produced according to the method described in the resin type dispersant (C2-47) of paragraph Nos. 0287 to 0289 of JP2019-133154A (dispersant having an ethylenically unsaturated bond-containing group; weight-average molecular weight: 19000. C═C value: 0.32 mmol/g)

Dispersant 4 to dispersant 9: dispersants 4 to 9 described in the following table (dispersant having an ethylenically unsaturated bond-containing group)

TABLE 8

| | Type | Type | Type | Molar ratio of each | | | | | |
|---|---|---|---|---|---|---|---|---|---|
| | of | of | of | constitutional unit | | | Weight- | | |
| | con- | con- | con- | Con- | Con- | Con- | average | | |
| | stitu- | stitu- | stitu- | stitu- | stitu- | stitu- | molec- | Acid | C═C |
| | tional | tional | tional | tional | tional | tional | ular | value | value |
| | unit | unit | unit | unit | unit | unit | weight | mgKOH/ | (mmol/ |
| | 1 | 2 | 3 | 1 | 2 | 3 | (Mw) | g) | g) |
| Dispersant 4 | 1-A | 2-A | 3-A | 5.0 | 87.0 | 8.0 | 8000 | 37 | 0.22 |
| Dispersant 5 | 1-A | 2-A | 3-E | 9.5 | 87.4 | 3.1 | 8200 | 39 | 0.14 |
| Dispersant 6 | 1-A | 2-A | 3-I | 9.5 | 87.4 | 3.1 | 8000 | 49 | 0.19 |
| Dispersant 7 | 1-A | 2-A | 3-I | 9.5 | 82.3 | 8.2 | 8000 | 49 | 0.51 |
| Dispersant 8 | 1-A | 2-A | 3-I | 9.0 | 78.3 | 12.7 | 8000 | 46 | 0.8 |
| Dispersant 9 | 1-A | 2-A | 3-I | 15.5 | 65.8 | 18.7 | 7500 | 80 | 1.2 |

Each constitutional unit described in the above table is as follows.

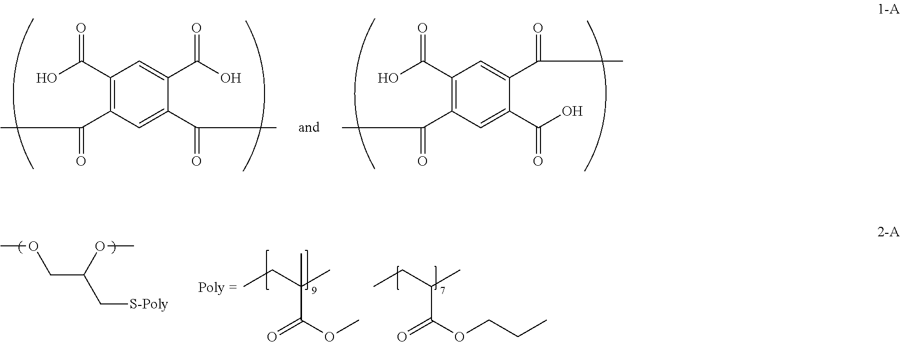

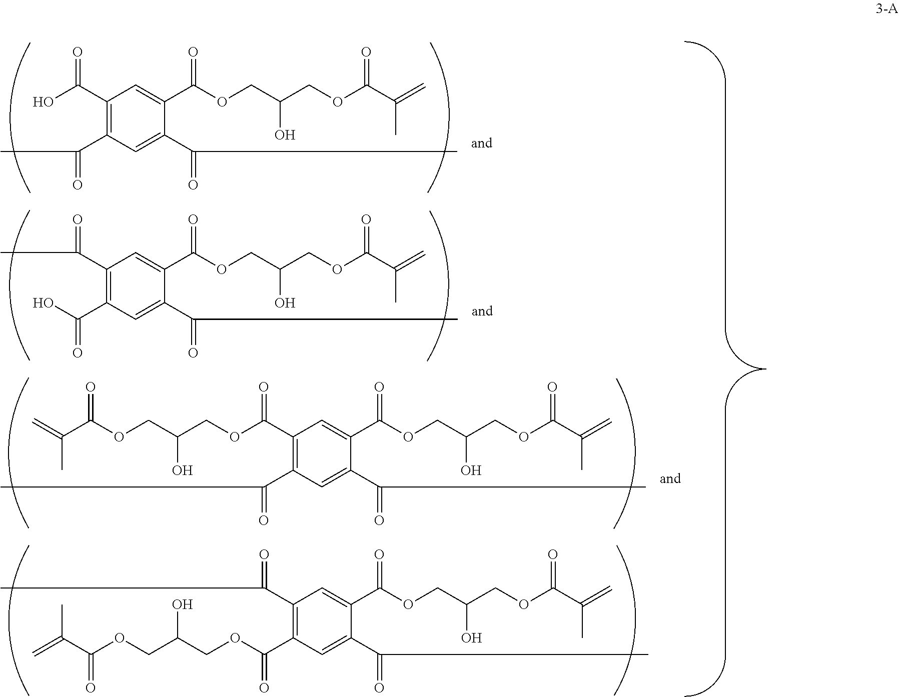

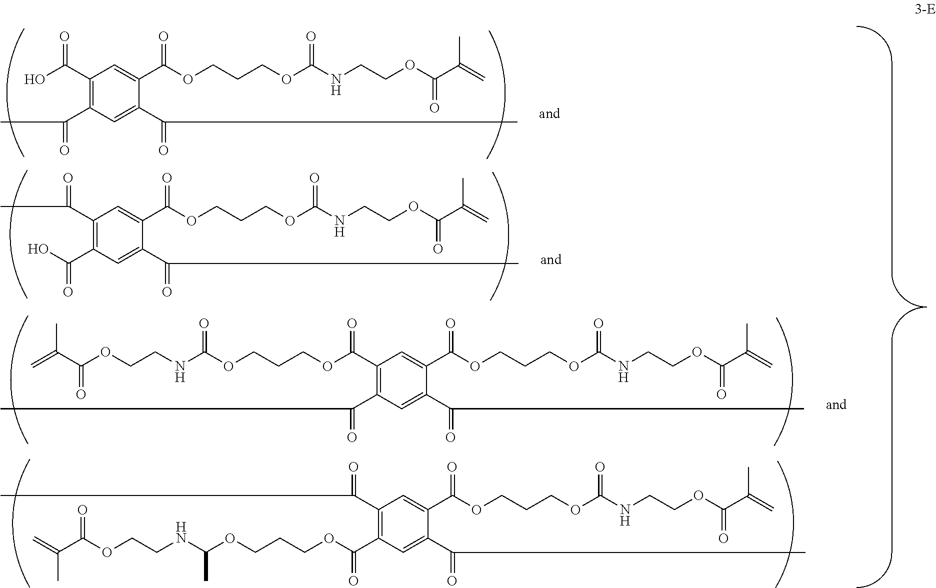

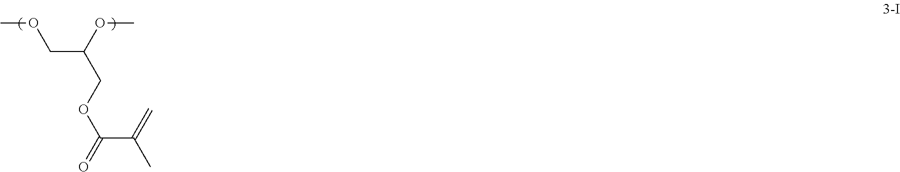

Dispersant 10: resin PA-1 synthesized by the following method (dispersant having an ethylenically unsaturated bond-containing group)

Dispersant 11: resin PA-19 synthesized by the following method (dispersant having an ethylenically unsaturated bond-containing group)

Dispersant 12: resin PB-1 synthesized by the following method (dispersant having an ethylenically unsaturated bond-containing group)

Dispersant 13: resin PB-17 synthesized by the following method (dispersant having an ethylenically unsaturated bond-containing group)

Dispersant 14: resin PB-18 synthesized by the following method (dispersant having an ethylenically unsaturated bond-containing group)

A macromonomer b-1 solution described later having a concentration (solid content) of 50% by mass as a monomer 2, a monomer a-1 as a monomer 1, and propylene glycol 1-monomethyl ether 2-acetate (PGMEA) were charged into a three-neck flask to obtain a mixture. The mixture was stirred while blowing nitrogen. Next, the mixture was warmed to 75° C. while allowing nitrogen flow into the flask. Next, dodecyl mercaptan (0.82 g), then 2,2′-azobis(methyl 2-methylpropionate) (0.43 g; hereinafter, also referred to as “V-601”) were added to the mixture to initiate a polymerization reaction. After heating the mixture at 75° C. for 2 hours, an additional V-601 (0.43 g) was added to the mixture. After 2 hours, an additional V-601 (0.43 g) was added to the mixture. After the reaction was further carried out for 2 hours, the mixture was heated to 90° C. and stirred for 3 hours. The polymerization reaction was terminated by the above operation. After terminating the reaction, dimethyldodecylamine as an amine compound (f-1) and 2,2,6,6-tetramethylpiperidine 1-oxyl (q-1, TEMPO) as a polymerization inhibitor were added thereto under air, and 4-hydroxybutyl acrylate glycidyl ether (monomer c-1) as a reactive compound was added dropwise thereto. After completion of the dropwise addition, the reaction was continued in air at 90° C. for 24 hours, and then the completion of the reaction was confirmed by acid value measurement. PGMEA was added to the obtained mixture so as to form a 30 mass % solution, thereby obtaining a resin PA-1.

The amounts of the monomer b-1 (solid content in the solution), the monomer a-1, the monomer c-1, the amine compound f-1, and the polymerization inhibitor q-1 were as described in the following tables.

In addition, a resin PA-19 was synthesized by the same method as the method for synthesizing the resin PA-1, except that the types and blending amounts of raw materials used were changed to those described in the following tables. The monomer 3 was added to the monomer 1.

In addition, a resin PB-1, a resin PB-17, and a resin PB-18 each were synthesized by the same method as the method for synthesizing PA-1, except that the types and blending amounts of raw materials used were changed to those described in the following tables. In a case where the monomer 3 was added, the monomer 3 was further added to the mixture of the monomer 1, the monomer 2, and the monomer 4.

In the following tables, the unit of the numerical value described in the column of “Content” is “% by mass”. In addition, the components described as “-” in the tables were not used.

TABLE 9

| | | | | | | | | | | | | | Weight- | | | |
|---|---|---|---|---|---|---|---|---|---|---|---|---|---|---|---|---|
| | | | | | | | | | | | Polymer- | | average | | | |
| | Monomer | | Monomer | | Monomer | | Reactive | | Amine | | ization | | molec- | Acid | C═C | Amine |
| | 1 | | 2 | | 3 | | compound | | compound | | inhibitor | | ular | value | value | value |
| | | Con- | | Con- | | Con- | | Con- | | Con- | | Con- | weight | (mgKOH/ | (mmol/ | (mgKOH/ |
| Resin | Type | tent | Type | tent | Type | tent | Type | tent | Type | tent | Type | tent | (Mw) | g) | g) | g) |
| PA-1 | a-1 | 50.99 | b-1 | 30.39 | — | — | c-1 | 9.01 | f-1 | 9.60 | q-1 | 0.3 | 17200 | 70 | 0.45 | 0.45 |
| PA-19 | a-9 | 18.76 | — | — | c-4 | 54.18 | c-1 | 24.02 | f-7 | 3.04 | q-1 | 1 | 8900 | 55 | 1.20 | 0.30 |

TABLE 10

| | | | | | | | | | | | | | Weight- | | | |
|---|---|---|---|---|---|---|---|---|---|---|---|---|---|---|---|---|
| | | | | | | | | | | | Polymer- | | average | | | |
| | Monomer | | Monomer | | Monomer | | Reactive | | Monomer | | ization | | molec- | Acid | C═C | Amine |
| | 1 | | 2 | | 4 | | compound | | 3 | | inhibitor | | ular | value | value | value |
| | | Con- | | Con- | | Con- | | Con- | | Con- | | Con- | weight | (mgKOH/ | (mmol/ | (mgKOH/ |
| Resin | Type | tent | Type | tent | Type | tent | Type | tent | Type | tent | Type | tent | (Mw) | g) | g) | g) |
| PB-1 | a-1 | 55.50 | b-1 | 23.90 | d-1 | 8.59 | c-1 | 12.01 | — | — | q-1 | 0.3 | 13800 | 70 | 0.60 | 0.60 |
| PB-17 | a-2 | 35.33 | — | — | d-1 | 4.30 | c-1 | 20.02 | c-3 | 40.35 | q-2 | 0.3 | 18100 | 30 | 1.00 | 0.30 |
| PB-18 | a-1 | 63.40 | — | — | d-1 | 5.73 | c-1 | 3.00 | c-5 | 27.87 | q-1 | 0.3 | 16400 | 110 | 0.15 | 0.40 |

Details of each component described in the above tables are shown below.

[Monomer 1]
a-1: ARONIX M-5300, ω-carboxy-polycaprolactone monoacrylate (manufactured by TOAGOSEI CO., LTD.)
a-2: LIGHT ESTER HO-MS, 2-methacryloyloxyethyl succinic acid (manufactured by KYOEISHA CHEMICAL Co., LTD.)
a-9: methacrylic acid

[Monomer 2]
b-1: macromonomer having the following structure (weight-average molecular weight: 3000)

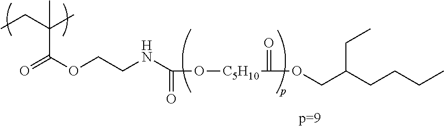

[Monomer 3]
e-3: 2-ethylhexyl methacrylate (manufactured by TOKYO CHEMICAL INDUSTRY CO., LTD.)
e-4: ARONIX M120 (manufactured by TOAGOSEI CO., LTD.), 2-(2-((2-ethylhexyl)oxy)ethoxy)ethyl acrylate
e-5: dicyclopentanyl methacrylate (manufactured by TOKYO CHEMICAL INDUSTRY CO., LTD.)

[Monomer 4]
d-1: 2-(dimethylamino)ethyl acrylate (manufactured by TOKYO CHEMICAL INDUSTRY CO., LTD.)

[Reactive Compound]
c-1: 4HBAGE, 4-hydroxybutyl acrylate glycidyl ether (manufactured by Nihon Kasei CO., LTD.)

[Amine Compound]
f-1: dimethyldodecylamine (manufactured by TOKYO CHEMICAL INDUSTRY CO., LTD.)
f-7: triethylamine (manufactured by TOKYO CHEMICAL INDUSTRY CO., LID.)

[Polymerization Inhibitor]
q-1: TEMPO free radical (2,2,6,6-tetramethylpiperidine 1-oxyl)
q-2: 4-hydroxy-TEMPO free radical (4-hydroxy-2,2,6,6-tetramethylpiperidine 2-oxyl)

[Dispersant not Including Ethylenically Unsaturated Bond-Containing Group]

Dispersant 101: resin compound having the following structure (acidic dispersant, weight-average molecular weight: 7000); a numerical value added to a main chain indicates a molar ratio of a repeating unit.

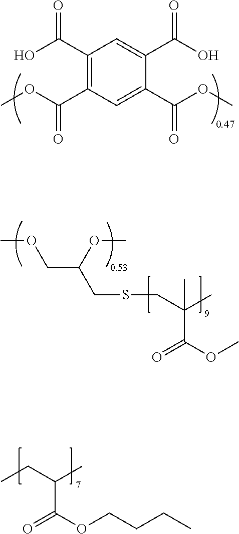

<Production of Photosensitive Composition>

Each material was mixed at a proportion of Formulations 1 to 5 shown below, and the obtained mixture was filtered through a nylon filter (manufactured by Pall Corporation) having a pore size of 0.45 μm to produce each photosensitive composition. Photosensitive compositions of Examples 1 to 59, and 82 to 123 are compositions for forming a green pixel, photosensitive compositions of Examples 60 to 81 are compositions for forming a red pixel, photosensitive compositions of Examples 124 to 127 are compositions for forming a blue pixel, photosensitive compositions of Examples 128 to 139 are compositions for forming a cyan pixel, photosensitive compositions of Examples 140 to 153 are compositions for forming a magenta pixel, photosensitive compositions of Examples 154 to 161 are compositions for forming a yellow pixel, photosensitive compositions of Examples 162 to 172 are compositions for forming a near infrared transmitting filter, and photosensitive compositions of Examples 173 to 175 are compositions for forming a near infrared cut filter.

(Formulation 1)

Dispersion liquid described in table—50.40 parts by mass
Binder resin described in table—3.84 parts by mass
Polymerizable monomer described in table—0.12 parts by mass
Photopolymerization initiator described in table—0.24 parts by mass
Epoxy compound 1—0.24 parts by mass
Surfactant 1—0.04 parts by mass
Polymerization inhibitor 1—0.0001 parts by mass
Solvent 1—45.12 parts by mass

(Formulation 2)

Dispersion liquid described in table—60.00 parts by mass
Binder resin described in table—2.40 parts by mass
Polymerizable monomer described in table—0.12 parts by mass
Photopolymerization initiator described in table—0.24 parts by mass
Epoxy compound 1—0.24 parts by mass
Surfactant 1—0.04 parts by mass
Polymerization inhibitor 1—0.0001 parts by mass
Solvent 1—36.96 parts by mass

(Formulation 3)

Dispersion liquid described in table 70.00 parts by mass
Binder resin described in table—0.90 parts by mass
Polymerizable monomer described in table—0.12 parts by mass
Photopolymerization initiator described in table—0.24 parts by mass
Epoxy compound 1—0.24 parts by mass
Surfactant 1—0.04 parts by mass
Polymerization inhibitor 1—0.0001 parts by mass
Solvent 1—28.46 parts by mass

(Formulation 4)

Dispersion liquid described in table—75.00 parts by mass
Binder resin described in table—0.15 parts by mass
Polymerizable monomer described in table—0.12 parts by mass
Photopolymerization initiator described in table—0.24 parts by mass
Epoxy compound 1—0.24 parts by mass
Surfactant 1—0.04 parts by mass
Polymerization inhibitor 1—0.0001 parts by mass
Solvent 1—24.21 parts by mass

(Formulation 5)

Dispersion liquid described in table—80.00 parts by mass
Binder resin described in table—0.36 parts by mass
Polymerizable monomer described in table—0.12 parts by mass
Photopolymerization initiator described in table—0.24 parts by mass
Epoxy compound 1—0.24 parts by mass
Surfactant 1—0.04 parts by mass
Polymerization inhibitor 1—0.0001 parts by mass
Solvent 1—19.00 parts by mass

Surfactant 1: mixture shown below (weight-average molecular weight: 14000); in the following formula, % representing the proportion of a repeating unit is % by mass.

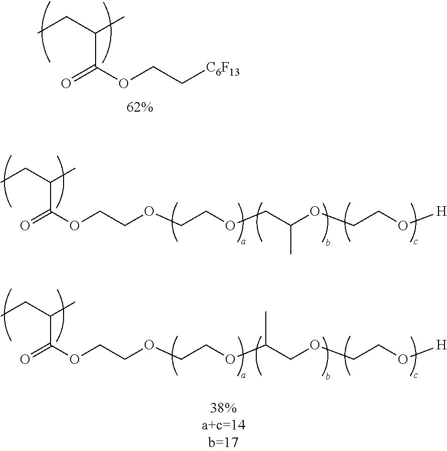

Epoxy compound 1: EHPE 3150 (manufactured by DAICEL-ALLNEX LTD.; 1,2-epoxy-4-(2-oxiranyl)cyclohexane adduct of 2,2′-bis(hydroxymethyl)-1-butanol)

Solvent 1: PGMEA

TABLE 11

| | | | | | | Total |
|---|---|---|---|---|---|---|
| | | | | | | content of |
| | | | | | | coloring |
| | | | | | Type of | material and |
| | | | Type | Type of | photoo | pigment |
| | | Type of | of | polymero | polymero | derivative |
| | Type of | dispersion | binder | izable | ization | (% by |
| | formulation | liquid | resin | monomer | initiator | mass) |
| Example 1 | Formulation 1 | Dispersion | Resin 1 | Monomer M1 | Initiator 1 | 50 |
| | | liquid 1-1 | | | | |
| Example 2 | Formulation 7 | Dispersion | Resin 1 | Monomer M1 | Initiator 1 | 60 |
| | | liquid 1-1 | | | | |
| Example 3 | Formulation 3 | Dispersion | Resin 1 | Monomer M1 | Initiator 1 | 70 |
| | | liquid 1-1 | | | | |
| Example 4 | Formulation 4 | Dispersion | Resin 1 | Monomer M1 | Initiator 1 | 75 |
| | | liquid 1-1 | | | | |
| Example 5 | Formulation 3 | Dispersion | Resin 1 | Monomer M1 | Initiator 1 | 70 |
| | | liquid 1-2 | | | | |
| Example 6 | Formulanon 1 | Dispersion | Resin 1 | Monomer M1 | Initiator 1 | 70 |
| | | liquid 1-3 | | | | |
| Example 7 | Formulation 3 | Dispersion | Resin 1 | Monomer M1 | Initiator 1 | 70 |
| | | liquid 1-4 | | | | |
| Example 8 | Formulation 3 | Dispersion | Resin 1 | Monomer M1 | Initiator 1 | 70 |
| | | liquid 1-5 | | | | |
| Example 9 | Formulation 3 | Dispersion | Resin 1 | Monomer M1 | Initiator 1 | 70 |
| | | liquid 1-6 | | | | |
| Example 10 | Formulation 3 | Dispersion | Resin 1 | Monomer M1 | Initiator 1 | 70 |
| | | liquid 1-7 | | | | |
| Example 11 | Formulation 3 | Dispersion | Resin 1 | Monomer M1 | Initiator 1 | 70 |
| | | liquid 1-8 | | | | |
| Example 12 | Formulation 3 | Dispersion | Resin 1 | Monomer M1 | Initiator 1 | 70 |
| | | liquid 1-9 | | | | |
| Example 13 | Formulation 3 | Dispersion | Resin 1 | Monomer M1 | Initiator 1 | 70 |
| | | liquid 1-10 | | | | |
| Example 14 | Formulation 3 | Dispersion | Resin 1 | Monomer M1 | Initiator 1 | 70 |
| | | liquid 1-11 | | | | |
| Example 15 | Formulation 3 | Dispersion | Resin 1 | Monomer M1 | Initiator 1 | 70 |
| | | liquid 1-12 | | | | |
| Example 16 | Formulation 3 | Dispersion | Resin 1 | Monomer M1 | Initiator 1 | 70 |
| | | liquid 1-13 | | | | |
| Example 17 | Formulation 3 | Dispersion | Resin 1 | Monomer M1 | Initiator 1 | 70 |
| | | liquid 1-14 | | | | |
| Example 18 | Formulation 3 | Dispersion | Resin 1 | Monomer M1 | Initiator 1 | 70 |
| | | liquid 1-15 | | | | |
| Example 19 | Formulation 3 | Dispersion | Resin 1 | Monomer M1 | Initiator 1 | 70 |
| | | liquid 1-16 | | | | |
| Example 20 | Formulation 3 | Dispersion | Resin 1 | Monomer M1 | Initiator 1 | 70 |
| | | liquid 1-17 | | | | |
| Example 21 | Formulation 3 | Dispersion | Resin 1 | Monomer M1 | Initiator 1 | 70 |
| | | liquid 1-18 | | | | |
| Example 22 | Formulation 3 | Dispersion | Resin 1 | Monomer M1 | Initiator 1 | 70 |
| | | liquid 1-19 | | | | |
| Example 23 | Formulation 3 | Dispersion | Resin 1 | Monomer M1 | Initiator 1 | 70 |
| | | liquid 1-20 | | | | |
| Example 24 | Formulation 3 | Dispersion | Resin 1 | Monomer M1 | Initiator 1 | 70 |
| | | liquid 1-21 | | | | |
| Example 25 | Formulation 3 | Dispersion | Resin 1 | Monomer M1 | Initiator 1 | 70 |
| | | liquid 1-22 | | | | |
| Example 26 | Formulation 3 | Dispersion | Resin 1 | Monomer M1 | Initiator 1 | 70 |
| | | liquid 1-23 | | | | |
| Example 27 | Formulation 3 | Dispersion | Resin 1 | Monomer M1 | Initiator 1 | 70 |
| | | liquid 1-24 | | | | |
| Example 28 | Formulation 3 | Dispersion | Resin 1 | Monomer M1 | Initiator 1 | 70 |
| | | liquid 1-25 | | | | |
| Example 29 | Formulation 3 | Dispersion | Resin 1 | Monomer M1 | Initiator 1 | 70 |
| | | liquid 1-26 | | | | |
| Example 30 | Formulation 3 | Dispersion | Resin 1 | Monomer M1 | Initiator 1 | 70 |
| | | liquid 1-27 | | | | |

TABLE 12

| | | | | | | Total |
|---|---|---|---|---|---|---|
| | | | | | | content of |
| | | | | | | coloring |
| | | | | | | material |
| | | | | | Type of | and |
| | Type | | | Type of | photo- | pigment |
| | of | Type of | Type of | polymer- | polymer- | derivative |
| | formu- | dispersion | binder | izable | ization | (% |
| | lation | liquid | resin | monomer | initiator | by mass) |
| Example 31 | Formu- | Dispersion | Resin 1 | Monomer M1 | Initiator 1 | 70 |
| | lation 3 | liquid 1-18 | | | | |
| Example 32 | Formu- | Dispersion | Resin 1 | Monomer M1 | Initiator 1 | 70 |
| | lation 3 | liquid 1-29 | | | | |
| Example 33 | Formu- | Dispersion | Resin 1 | Monomer M1 | Initiator 1 | 70 |
| | lation 3 | liquid 1-30 | | | | |
| Example 34 | Formu- | Dispersion | Resin 1 | Monomer M1 | Initiator 1 | 70 |
| | lation 3 | liquid 1-31 | | | | |
| Example 35 | Formu- | Dispersion | Resin 1 | Monomer M1 | Initiator 1 | 70 |
| | lation 3 | liquid 1-32 | | | | |
| Example 36 | Formu- | Dispersion | Resin 1 | Monomer M1 | Initiator 1 | 70 |
| | lation 3 | liquid 1-33 | | | | |
| Example 37 | Formu- | Dispersion | Resin 1 | Monomer M1 | Initiator 1 | 70 |
| | lation 3 | liquid 1-34 | | | | |
| Example 38 | Formu- | Dispersion | Resin 1 | Monomer M1 | Initiator 1 | 70 |
| | lation 3 | liquid 1-35 | | | | |
| Example 39 | Formu- | Dispersion | Resin 1 | Monomer M1 | Initiator 1 | 70 |
| | lation 3 | liquid 1-36 | | | | |
| Example 40 | Formu- | Dispersion | Resin 1 | Monomer M1 | Initiator 1 | 70 |
| | lation 3 | liquid 1-37 | | | | |
| Example 41 | Formu- | Dispersion | Resin 1 | Monomer M1 | Initiator 1 | 70 |
| | lation 3 | liquid 1-38 | | | | |
| Example 41 | Formu- | Dispersion | Resin 1 | Monomer M1 | Initiator 1 | 70 |
| | lation 3 | liquid 1-39 | | | | |
| Example 43 | Formu- | Dispersion | Resin 1 | Monomer M1 | Initiator 1 | 70 |
| | lation 3 | liquid 1-40 | | | | |
| Example 44 | Formu- | Dispersion | Resin 1 | Monomer M1 | Initiator 1 | 70 |
| | lation 3 | liquid 1-41 | | | | |
| Example 45 | Formu- | Dispersion | Resin 1 | Monomer M1 | Initiator 1 | 70 |
| | lation 3 | liquid 1-41 | | | | |
| Example 46 | Formu- | Dispersion | Resin 1 | Monomer M1 | Initiator 1 | 70 |
| | lation 3 | liquid 1-43 | | | | |
| Example 47 | Formu- | Dispersion | Resin 2 | Monomer M1 | Initiator 1 | 70 |
| | lation 3 | liquid 1-1 | | | | |
| Example 48 | Formu- | Dispersion | Resin 1 | Monomer M1 | Initiator 1 | 70 |
| | lation 3 | liquid 1-I | | | | |
| Example 49 | Formu- | Dispersion | Resin 1 | Monomer M1 | Initiator 2 | 70 |
| | lation 3 | liquid 1-1 | | | | |
| Example 50 | Formu- | Dispersion | Resin 1 | Monomer M1 | Initiator | |
| | lation 3 | liquid 1-1 | | | 1/initiator | 70 |
| | | | | | 2 [symbol] 5/5 | |
| | | | | | (mass ratio) | |
| Example 51 | Formu- | Dispersion | Resin 1 | Monomer M1 | Initiator 1 | 70 |
| | lation 3 | liquid 1-44 | | | | |
| Example 52 | Formu- | Dispersion | Resin 1 | Monomer M1 | Initiator 1 | 70 |
| | lation 3 | liquid 1-45 | | | | |
| Example 53 | Formu- | Dispersion | Resin 1/ | Monomer M1 | Initiator 1 | 70 |
| | lation 3 | liquid 1-1 | resin 2 [symbol] | | | |
| | | | 5/5 (mass | | | |
| | | | ratio) | | | |
| Example 54 | Formu- | Dispersion | Resin 1 | Monomer | Initiator 2 | 70 |
| | lation 3 | liquid 1-1 | | M1/Monomer | | |
| | | | | M2 3/7 | | |
| | | | | (mass ratio) | | |
| Example 55 | Formu- | Dispersion | Resin 1 | Monomer M1 | Initiator 1 | 70 |
| | lation 3 | liquid 1-40 | | | | |
| Example 56 | Formu- | Dispersion | Resin 1 | Monomer M1 | Initiator 1 | 70 |
| | lation 3 | liquid 1-47 | | | | |
| Example 57 | Formu- | Dispersion | Resin 1 | Monomer M1 | Initiator 3 | 70 |
| | lation 3 | liquid 1-1 | | | | |
| Example 58 | Formu- | Dispersion | Resin 1 | Monomer | Initiator 3 | 70 |
| | lation 3 | liquid 1-1 | | M3/monomer | | |
| | | | | M4 [symbol] 6/4 | | |
| | | | | (mass ratio) | | |
| Example 59 | Formu- | Dispersion | Resin 1 | Monomer M1 | Initiator 1 | 80 |
| | lation 5 | liquid 2-1 | | | | |

[symbol]

indicates data missing or illegible when filed

TABLE 13

| | | | | | | Total |
|---|---|---|---|---|---|---|
| | | | | | | content of |
| | | | | | Type of | material |
| | | | | | coloring | and |
| | | | Type | Type of | photo- | pigment |
| | | | of | polymer- | polymer- | derivative |
| | Type or | Type of | binder | izable | ization | (% |
| | formulation | dispersion liquid | resin | monomer | initiator | by mass) |
| Example 60 | Formulation 3 | Dispersion liquid 3-1 | Resin 1 | Monomer M1 | Initiator 1 | 70 |
| Example 61 | Formulation 3 | Dispersion liquid 3-2 | Resin 1 | Monomer M1 | Initiator 1 | 70 |
| Example 62 | Formulation 3 | Dispersion liquid 3-3 | Resin 1 | Monomer M1 | Initiator 1 | 70 |
| Example 63 | Formulation 3 | Dispersion liquid 3-4 | Resin 1 | Monomer M1 | Initiator 1 | 70 |
| Example 64 | Formulation 3 | Dispersion liquid 3-5 | Resin 1 | Monomer M1 | Initiator 1 | 70 |
| Example 65 | Formulation 3 | Dispersion liquid 3-6 | Resin 1 | Monomer M1 | Initiator 1 | 70 |
| Example 66 | Formulation 3 | Dispersion liquid 3-7 | Resin 1 | Monomer M1 | Initiator 1 | 70 |
| Example 67 | Formulation 3 | Dispersion liquid 3-8 | Resin 1 | Monomer M1 | Initiator 1 | 70 |
| Example 68 | Formulation 3 | Dispersion liquid 3-9 | Resin 1 | Monomer M1 | Initiator 1 | 70 |
| Example 69 | Formulation 3 | Dispersion liquid 3-10 | Resin 1 | Monomer M1 | Initiator 1 | 70 |
| Example 70 | Formulation 3 | Dispersion liquid 3-11 | Resin 1 | Monomer M1 | Initiator 1 | 70 |
| Example 71 | Formulation 3 | Dispersion liquid 3-12 | Resin 1 | Monomer M1 | Initiator 1 | 70 |
| Example 72 | Formulation 3 | Dispersion liquid 3-13 | Resin 1 | Monomer M1 | Initiator 1 | 70 |
| Example 73 | Formulation 3 | Dispersion liquid 3-14 | Resin 1 | Monomer M1 | Initiator 1 | 70 |
| Example 74 | Formulation 3 | Dispersion liquid 3-15 | Resin 1 | Monomer M1 | Initiator 1 | 70 |
| Example 75 | Formulation 3 | Dispersion liquid 3-16 | Resin 1 | Monomer M1 | Initiator 1 | 70 |
| Example 76 | Formulation 3 | Dispersion liquid 3-17 | Resin 1 | Monomer M1 | Initiator 1 | 70 |
| Example 77 | Formulation 3 | Dispersion liquid 3-18 | Resin 1 | Monomer M1 | Initiator 1 | 70 |
| Example 78 | Formulation 3 | Dispersion liquid 3-19 | Resin 1 | Monomer M1 | Initiator 1 | 70 |
| Example 79 | Formulation 3 | Dispersion liquid 3-20 | Resin 1 | Monomer M1 | Initiator 1 | 70 |
| Example 80 | Formulation 3 | Dispersion liquid 3-21 | Resin 1 | Monomer M1 | Initiator 1 | 70 |
| Example 81 | Formulation 3 | Dispersion liquid 3-22 | Resin 1 | Monomer M1 | Initiator 1 | 70 |
| Example 82 | Formulation 3 | Dispersion liquid 3-23 | Resin 1 | Monomer M1 | Initiator 1 | 70 |
| Example 83 | Formulation 3 | Dispersion liquid 3-24 | Resin 1 | Monomer M1 | Initiator 1 | 70 |
| Example 84 | Formulation 3 | Dispersion liquid 3-25 | Resin 1 | Monomer M1 | Initiator 1 | 70 |
| Example 85 | Formulation 3 | Dispersion liquid 1-26 | Resin 1 | Monomer M1 | Initiator 1 | 70 |
| Example 86 | Formulation 3 | Dispersion liquid 3-27 | Resin 1 | Monomer M1 | Initiator 1 | 70 |
| Example 87 | Formulation 3 | Dispersion liquid 3-28 | Resin 1 | Monomer M1 | Initiator 1 | 70 |
| Example 88 | Formulation 3 | Dispersion liquid 3-29 | Resin 1 | Monomer M1 | Initiator 1 | 70 |
| Example 89 | Formulation 3 | Dispersion liquid 3-30 | Resin 1 | Monomer M1 | Initiator 1 | 70 |
| Example 90 | Formulation 3 | Dispersion liquid 3-31 | Resin 1 | Monomer M1 | Initiator 1 | 70 |
| Example 91 | Formulation 3 | Dispersion liquid 3-32 | Resin 1 | Monomer M1 | Initiator 1 | 70 |
| Example 92 | Formulation 3 | Dispersion liquid 3-31 | Resin 1 | Monomer M1 | Initiator 1 | 70 |
| Example 93 | Formulation 3 | Dispersion liquid 3-34 | Resin 1 | Monomer M1 | Initiator 1 | 70 |
| Example 94 | Formulation 3 | Dispersion liquid 3-35 | Resin 1 | Monomer M1 | Initiator 1 | 70 |
| Example 95 | Formulation 3 | Dispersion liquid 3-36 | Resin 1 | Monomer M1 | Initiator 1 | 70 |
| Example 96 | Formulation 3 | Dispersion liquid 3-37 | Resin 1 | Monomer M1 | Initiator 1 | 70 |
| Example 97 | Formulation 3 | Dispersion liquid 3-38 | Resin 1 | Monomer M1 | Initiator 1 | 70 |
| Example 98 | Formulation 3 | Dispersion liquid 3-39 | Resin 1 | Monomer M1 | Initiator 1 | 70 |
| Example 99 | Formulation 3 | Dispersion liquid 3-40 | Resin 1 | Monomer M1 | Initiator 1 | 70 |
| Example 100 | Formulation 3 | Dispersion liquid 3-41 | Resin 1 | Monomer M1 | Initiator 1 | 70 |
| Example 101 | Formulation 3 | Dispersion liquid 3-42 | Resin 1 | Monomer M1 | Initiator 1 | 70 |
| Example 102 | Formulation 3 | Dispersion liquid 3-43 | Resin 1 | Monomer M1 | Initiator 1 | 70 |
| Example 103 | Formulation 3 | Dispersion liquid 3-44 | Resin 1 | Monomer M1 | Initiator 1 | 70 |
| Example 104 | Formulation 3 | Dispersion liquid 3-45 | Resin 1 | Monomer M1 | Initiator 1 | 70 |
| Example 105 | Formulation 3 | Dispersion liquid 3-46 | Resin 1 | Monomer M1 | Initiator 1 | 70 |
| Example 106 | Formulation 3 | Dispersion liquid 3-47 | Resin 1 | Monomer M1 | Initiator 1 | 70 |
| Example 107 | Formulation 3 | Dispersion liquid 3-48 | Resin 1 | Monomer M1 | Initiator 1 | 70 |
| Example 108 | Formulation 3 | Dispersion liquid 3-49 | Resin 1 | Monomer M1 | Initiator 1 | 70 |
| Example 109 | Formulation 3 | Dispersion liquid 3-50 | Resin 1 | Monomer M1 | Initiator 1 | 70 |
| Example 110 | Formulation 3 | Dispersion liquid 3-51 | Resin 1 | Monomer M1 | Initiator 1 | 70 |
| Example 111 | Formulation 3 | Dispersion liquid 3-52 | Resin 1 | Monomer M1 | Initiator 1 | 70 |
| Example 112 | Formulation 3 | Dispersion liquid 3-53 | Resin 1 | Monomer M1 | Initiator 1 | 70 |
| Example 113 | Formulation 3 | Dispersion liquid 3-54 | Resin 1 | Monomer M1 | Initiator 1 | 70 |
| Example 114 | Formulation 3 | Dispersion liquid 3-55 | Resin 1 | Monomer M1 | Initiator 1 | 70 |
| Example 115 | Formulation 3 | Dispersion liquid 3-56 | Resin 1 | Monomer M1 | Initiator 1 | 70 |

TABLE 14

| | | | | | | Total |
|---|---|---|---|---|---|---|
| | | | | | | content of |
| | | | | | Type of | material |
| | | | | | coloring | and |
| | | | Type | Type of | photo- | pigment |
| | | | of | polymer- | polymer- | derivative |
| | Type or | Type of | binder | izable | ization | (% |
| | formulation | dispersion liquid | resin | monomer | initiator | by mass) |
| Example 116 | Formulation 3 | Dispersion liquid 3-57 | Resin 1 | Monomer M1 | Initiator 1 | 70 |
| Example 117 | Formulation 3 | Dispersion liquid 3-58 | Resin 1 | Monomer M1 | Initiator 1 | 70 |
| Example 118 | Formulation 3 | Dispersion liquid 3-59 | Resin 1 | Monomer M1 | Initiator 1 | 70 |
| Example 119 | Formulation 3 | Dispersion liquid 3-60 | Resin 1 | Monomer M1 | Initiator 1 | 70 |
| Example 120 | Formulation 3 | Dispersion liquid 3-61 | Resin 1 | Monomer M1 | Initiator 1 | 70 |
| Example 121 | Formulation 3 | Dispersion liquid 3-62 | Resin 1 | Monomer M1 | Initiator 1 | 70 |
| Example 122 | Fomtulation 3 | Dispersion liquid 3-63 | Resin 1 | Monomer M1 | Initiator 1 | 70 |
| Example 123 | Formulation 3 | Dispersion liquid 3-64 | Resin 1 | Monomer M1 | Initiator 1 | 70 |
| Example 124 | Formulation 3 | Dispersion liquid 3-65 | Resin 1 | Monomer M1 | Initiator 1 | 70 |
| Example 125 | Formulation 3 | Dispersion liquid 3-66 | Resin 1 | Monomer M1 | Initiator 1 | 70 |
| Example 126 | Formulation 3 | Dispersion liquid 3-67 | Resin 1 | Monomer M1 | Initiator 1 | 70 |
| Example 127 | Formulation 3 | Dispersion liquid 3-68 | Resin 1 | Monomer M1 | Initiator 1 | 70 |
| Example 128 | Formulation 3 | Dispersion liquid 3-69 | Resin 1 | Monomer M1 | Initiator 1 | 70 |
| Example 129 | Formulation 3 | Dispersion liquid 3-70 | Resin 1 | Monomer M1 | Initiator 1 | 70 |
| Example 130 | Formulation 3 | Dispersion liquid 3-71 | Resin 1 | Monomer M1 | Initiator 1 | 70 |
| Example 131 | Formulation 3 | Dispersion liquid 3-72 | Resin 1 | Monomer M1 | Initiator 1 | 70 |
| Example 132 | Formulation 3 | Dispersion liquid 3-73 | Resin 1 | Monomer M1 | Initiator 1 | 70 |
| Example 133 | Formulation 3 | Dispersion liquid 3-74 | Resin 1 | Monomer M1 | Initiator 1 | 70 |
| Example 134 | Formulation 3 | Dispersion liquid 3-75 | Resin 1 | Monomer M1 | Initiator 1 | 70 |
| Example 135 | Formulation 3 | Dispersion liquid 3-76 | Resin 1 | Monomer M1 | Initiator 1 | 70 |
| Example 136 | Formulation 3 | Dispersion liquid 3-77 | Resin 1 | Monomer M1 | Initiator 1 | 70 |
| Example 137 | Formulation 3 | Dispersion liquid 3-78 | Resin 1 | Monomer M1 | Initiator 1 | 70 |
| Example 138 | Formulation 3 | Dispersion liquid 3-79 | Resin 1 | Monomer M1 | Initiator 1 | 70 |
| Example 139 | Formulation 3 | Dispersion liquid 3-80 | Resin 1 | Monomer M1 | Initiator 1 | 70 |
| Example 140 | Formulation 3 | Dispersion liquid 3-81 | Resin 1 | Monomer M1 | Initiator 1 | 70 |
| Example 141 | Formulation 3 | Dispersion liquid 3-82 | Resin 1 | Monomer M1 | Initiator 1 | 70 |
| Example 142 | Formulation 3 | Dispersion liquid 3-83 | Resin 1 | Monomer M1 | Initiator 1 | 70 |
| Example 143 | Formulation 3 | Dispersion liquid 3-84 | Resin 1 | Monomer M1 | Initiator 1 | 70 |
| Example 144 | Formulation 3 | Dispersion liquid 3-85 | Resin 1 | Monomer M1 | Initiator 1 | 70 |
| Example 145 | Formulation 3 | Dispersion liquid 3-86 | Resin 1 | Monomer M1 | Initiator 1 | 70 |
| Example 146 | Formulation 3 | Dispersion liquid 3-87 | Resin 1 | Monomer M1 | Initiator 1 | 70 |
| Example 147 | Formulation 3 | Dispersion liquid 3-88 | Resin 1 | Monomer M1 | Initiator 1 | 70 |
| Example 148 | Formulation 3 | Dispersion liquid 3-89 | Resin 1 | Monomer M1 | Initiator 1 | 70 |
| Example 149 | Formulation 3 | Dispersion liquid 3-90 | Resin 1 | Monomer M1 | Initiator 1 | 70 |
| Example 150 | Formulation 3 | Dispersion liquid 3-91 | Resin 1 | Monomer M1 | Initiator 1 | 70 |
| Example 151 | Formulation 3 | Dispersion liquid 3-92 | Resin 1 | Monomer M1 | Initiator 1 | 70 |
| Example 152 | Fortnulation 3 | Dispersion liquid 3-91 | Resin 1 | Monomer M1 | Initiator 1 | 70 |
| Example 153 | Formulation 3 | Dispersion liquid 3-94 | Resin 1 | Monomer M1 | Initiator 1 | 70 |
| Example 154 | Formulation 3 | Dispersion liquid 3-95 | Resin 1 | Monomer M1 | Initiator 1 | 70 |
| Example 155 | Formulation 3 | Dispersion liquid 3-96 | Resin 1 | Monomer M1 | Initiator 1 | 70 |
| Example 156 | Formulation 3 | Dispersion liquid 3-97 | Resin 1 | Monomer M1 | Initiator 1 | 70 |
| Example 157 | Formulation 3 | Dispersion liquid 3-98 | Resin 1 | Monomer M1 | Initiator 1 | 70 |
| Example 158 | Formulation 3 | Dispersion liquid 3-99 | Resin 1 | Monomer M1 | Initiator 1 | 70 |
| Example 159 | Formulation 3 | Dispersion liquid 3-100 | Resin 1 | Monomer M1 | Initiator 1 | 70 |
| Example 160 | Formulation 3 | Dispersion liquid 3-101 | Resin 1 | Monomer M1 | Initiator 1 | 70 |
| Example 161 | Formulation 3 | Dispersion liquid 3-102 | Resin 1 | Monomer M1 | Initiator 1 | 70 |
| Example 162 | Formulation 3 | Dispersion liquid 3-103 | Resin 1 | Monomer M1 | Initiator 1 | 70 |
| Example 163 | Formulation 3 | Dispersion liquid 3-104 | Resin 1 | Monomer M1 | Initiator 1 | 70 |
| Example 164 | Formulation 3 | Dispersion liquid 3-105 | Resin 1 | Monomer M1 | Initiator 1 | 70 |
| Example 165 | Formulation 3 | Dispersion liquid 3-106 | Resin 1 | Monomer M1 | Initiator 1 | 70 |
| Example 166 | Formulation 3 | Dispersion liquid 3-107 | Resin 1 | Monomer M1 | Initiator 1 | 70 |
| Example 167 | Formulation 3 | Dispersion liquid 3-108 | Resin 1 | Monomer M1 | Initiator 1 | 70 |
| Example 168 | Fonnulation 3 | Dispersion liquid 3-109 | Resin 1 | Monomer M1 | Initiator 1 | 70 |
| Example 169 | Formulation 3 | Dispersion liquid 3-110 | Resin 1 | Monomer M1 | Initiator 1 | 70 |
| Example 170 | Formulation 3 | Dispersion liquid 3-111 | Resin 1 | Monomer M1 | Initiator 1 | 70 |
| Example 171 | Formulation 3 | Dispersion liquid 3-112 | Resin 1 | Monomer M1 | Initiator 1 | 70 |
| Example 172 | Formulation 3 | Dispersion liquid 3-113 | Resin 1 | Monomer M1 | Initiator 1 | 70 |
| Example 173 | Formulation 3 | Dispersion liquid 3-114 | Resin 1 | Monomer M1 | Initiator 1 | 70 |
| Example 174 | Formulation 3 | Dispersion liquid 3-115 | Resin 1 | Monomer M1 | Initiator 1 | 70 |
| Example 175 | Formulation 3 | Dispersion liquid 3-116 | Resin 1 | Monomer M1 | Initiator 1 | 70 |

TABLE 15

| | | | | | | Total |
|---|---|---|---|---|---|---|
| | | | | | Type of | content of |
| | | | | | photo- | coloring |
| | | | Type | Type of | poly- | material and |
| | | Type of | of | polymer- | mer- | pigment |
| | Type of | dispersion | binder | izable | ization | derivative |
| | formulation | liquid | resin | monomer | initiator | (% by mass) |
| Comparative | Formulation | Dispersion | Resin | Monomer | Initiator | 50 |
| example 1 | 1 | liquid r1-1 | 1 | M1 | 1 | |
| Comparative | Formulation | Dispersion | Resin | Monomer | Initiator | 60 |
| example 2 | 2 | liquid r1-1 | 1 | M1 | 1 | |
| Comparative | Formulation | DisPersion | Resin | Monomer | Initiator | 70 |
| example 3 | 3 | liquid r1-1 | 1 | M1 | 1 | |
| Comparative | Formulation | Dispersion | Resin | Monomer | Initiator | 75 |
| example 1 | 4 | liquid r1-1 | 1 | M1 | 1 | |
| Comparative | Formulation | Dispersion | Resin | Monomer | Initiator | 70 |
| example 5 | 3 | liquid r1-2 | 1 | M1 | 1 | |
| Comparative | Formulation | Dispersion | Resin | Monomer | Initiator | 70 |
| example 6 | 3 | liquid r1-3 | 1 | M1 | 1 | |
| Comparative | Formulation | Dispersion | Resin | Monomer | Initiator | 70 |
| example 7 | 3 | liquid r1-4 | 1 | M1 | 1 | |
| Comparatr e | Formulation | Dispersion | Resin | Monomer | Initiator | 70 |
| example 8 | 3 | liquid r1-5 | 1 | M1 | 1 | |
| Comparative | Formulation | Dispersion | Resin | Monomer | Initiator | 70 |
| example 9 | 3 | liquid r1-6 | 1 | M1 | 1 | |
| Comparative | Formulation | Dispersion | Resin | Monomer | Initiator | 70 |
| example 10 | 3 | liquid r1-7 | 1 | M1 | 1 | |
| Comparative | Formulation | Dispersion | Resin | Monomer | Initiator | 70 |
| example 11 | 3 | liquid r1-8 | 1 | M1 | 1 | |
| Comparative | Formulation | Dispersion | Resin | Monomer | Initiator | 70 |
| example 12 | 3 | liquid r1-9 | 1 | M1 | 1 | |
| Comparative | Formulation | Dispersion | Resin | Monomer | Initiator | 70 |
| example 13 | 3 | liquid r1-10 | 1 | M1 | 1 | |
| Comparative | Formulation | Dispersion | Resin | Monomer | Initiator | 80 |
| example 14 | 5 | liquid r2-1 | 1 | M1 | 1 | |

The details of the materials indicated by the abbreviations in the tables showing the formulation of the photosensitive compositions described above are as follows.

(Binder Resin)

Resin 1: resin having the following structure (weight-average molecular weight: 20000); a numerical value added to a main chain indicates a molar ratio of a repeating unit.

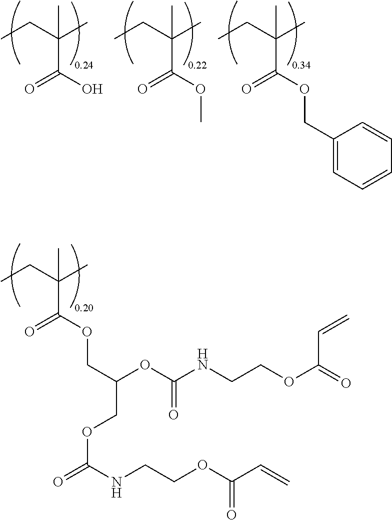

Resin 2: resin having the following structure (weight-average molecular weight: 110000): a numerical value added to a main chain indicates a molar ratio of a repeating unit.

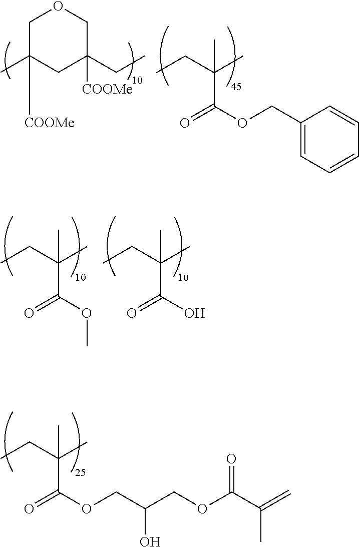

(Polymerizable Monomer)

Monomer M1: compound having the following structure

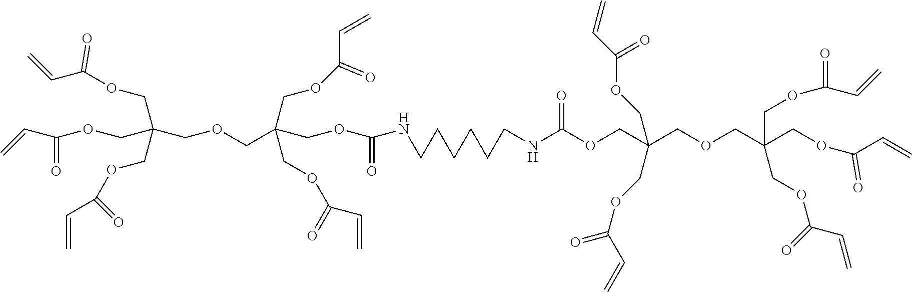 [SMILES: C=CC(=O)OCC(COCC(COC(=O)C=C)(COC(=O)C=C)COC(=O)NCCCCCCNC(=O)OCC(COCC(COC(=O)C=C)(COC(=O)C=C)COC(=O)C=C)(COC(=O)C=C)COC(=O)C=C)(COC(=O)C=C)COC(=O)C=C]

Monomer M2: mixture of compounds having the following structures (molar ratio of a compound having the structure on the left side to a compound having the structure on the right side=7:3)

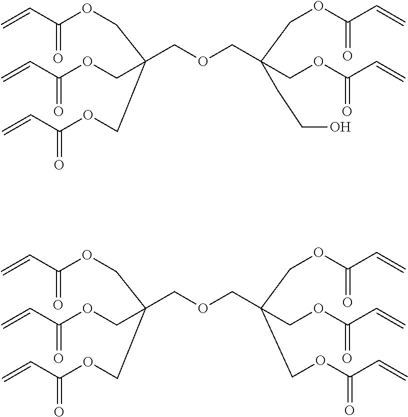

Monomer M3: trimethylolpropane ethyleneoxy-modified triacrylate (manufactured by TOAGOSEI CO., LID, ARONIX M-350)

Monomer M4: pentaerythritol tetraacrylate (manufactured by Shin-Nakamura Chemical Co., Ltd., NK ESTER A-TMMT)

(Photopolymerization Initiator)

Initiator 1: compound having the following structure

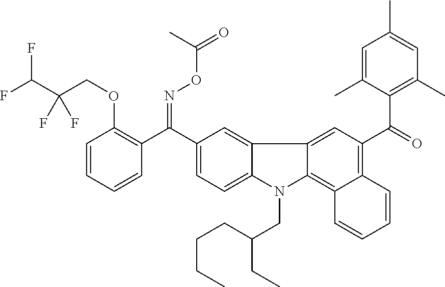 [SMILES: CCCCC(CC)Cn1c2ccc(/C(=N\OC(C)=O)c3ccccc3OCC(F)(F)C(F)F)cc2c2cc(C(=O)c3c(C)cc(C)cc3C)c3ccccc3c21]

Initiator 2: compound having the following structure

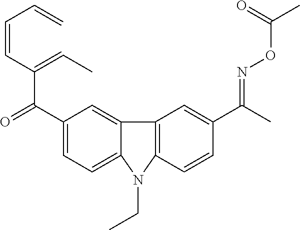 [SMILES: C=C/C=C\C(=C\C)C(=O)c1ccc2c(c1)c1cc(/C(C)=N/OC(C)=O)ccc1n2CC]

Initiator 3: compound having the following structure

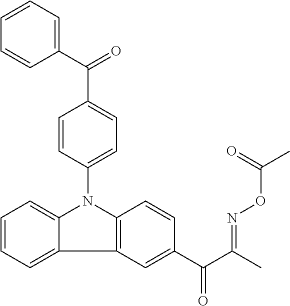 [SMILES: CC(=O)O/N=C(\C)C(=O)c1ccc2c(c1)c1ccccc1n2-c1ccc(C(=O)c2ccccc2)cc1]

<Evaluation 1> Pattern Line Width Stability

A composition for a base layer was applied to a silicon wafer having a diameter of 8 inch (=20.32 cm) by a spin coating method. Next, the composition was heated using a hot plate at 100° C. for 2 minutes and was further heated using a hot plate at 230° C. for 2 minutes. As a result, a base layer having a film thickness of 10 nm was formed. The composition for a base layer will be described later.

Next, each photosensitive composition was applied to the silicon wafer on which the base layer had been formed by a spin coating method so that a film thickness after film formation was 0.4 μm, and the curable composition was heated using a hot plate at 100° C. for 2 minutes. Next, using an i-ray stepper exposure device FPA-3000 i5+ (manufactured by Canon Inc.), the coating film was exposed through a mask having a 0.8 μm island pattern at an exposure amount of 150 mJ/cm². Next, the coating film was left for 30 minutes (PED1) or 72 hours (PED2) in an environment with a temperature of 23° C. and a humidity of 50%, and puddle development was performed at 23° C. for 60 seconds using a 0.3% by mass of tetramethylammonium hydroxide (TMAH) aqueous solution. Thereafter, the coating film was rinsed with a spin shower, washed with pure water, and heated using a hot plate at 220° C. for 5 minutes to form an island pattern.

A line width (size) of the obtained pattern was compared between the case where the island pattern was formed by performing exposure in an environment with a temperature of 23° C. and a humidity of 50%, and performing development after 30 minutes (PED1) and the case where the island pattern was formed by performing exposure in an environment with a temperature of 23° C. and a humidity of 50%, and performing development after 72 hours (PED2), thereby evaluating pattern line width stability. S-9260A (manufactured by Hitachi High-Tech Fielding Corporation) was used for measuring the line width of the pattern.

(Determination Criterion)

Δ=|Line width of island pattern formed under PED1 condition−Line width of island pattern formed under PED2 condition|

A: Δ<0.01 μm

B: 0.01 μm≤Δ<0.03 μm

C: 0.03 μm≤Δ<0.07 μm

D: 0.07 μm≤Δ<0.15 μm

E: 0.15 μm≤Δ

The composition for a base layer was produced by mixing the following raw materials.

Resin A—0.7 parts by mass
Surfactant A—0.8 parts by mass

Propylene glycol monomethyl ether acetate (PGMEA) . . . 98.5 parts by mass

The details of the raw materials are as follows.
Resin A: CYCLOMER P (ACA) 230AA (manufactured by DAICEL-ALLNEX LTD.; acid value=30 mgKOH/g, Mw=15000, 54% by mass PGME solution)
Surfactant A: 0.2% by mass PGMEA solution of a compound having the following structure (Mw=14000; numerical value “%” indicating the proportion of a repeating unit is mol %; fluorine-based surfactant)

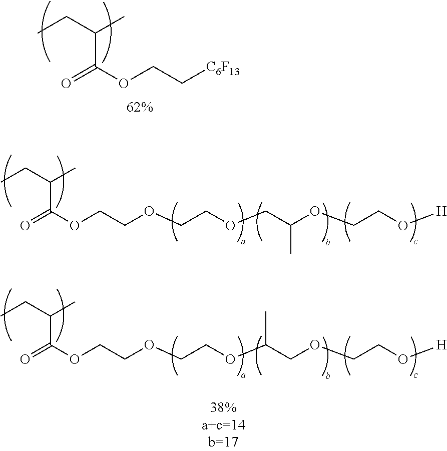

<Evaluation 2> Evaluation of Defects

The above-described composition for a base layer was applied to a silicon wafer having a diameter of 8 inch (=20.32 cm) by a spin coating method. Next, the composition was heated using a hot plate at 100° C. for 2 minutes and was further heated using a hot plate at 230° C. for 2 minutes. As a result, a base layer having a film thickness of 10 nm was formed. Next, each photosensitive composition was applied to the silicon wafer on which the base layer had been formed by a spin coating method so that a film thickness after film formation was 0.4 μm, and the curable composition was heated using a hot plate at 100° C. for 2 minutes. Next, using an i-ray stepper exposure device FPA-3000 i5+ (manufactured by Canon Inc.), the coating film was exposed through a mask having a 0.8 μm island pattern at an exposure amount of 150 mJ/cm². Next, the coating film was left for 30 minutes in an environment with a temperature of 23° C. and a humidity of 50%, and puddle development was performed at 23° C. for 60 seconds using a 0.3% by mass of tetramethylammonium hydroxide (TMAH) aqueous solution. Thereafter, the coating film was rinsed with a spin shower, washed with pure water, and heated using a hot plate at 220° C. for 5 minutes to form an island pattern.

After performing a constant temperature and humidity test (temperature: 60° C., humidity: 85%, 2000 hours) on the silicon wafer on which the obtained pattern had been formed, 20 points on the surface of the silicon wafer were observed with an optical microscope to confirm the presence or absence of foreign matters in the film.

A: no foreign matter was observed after the constant temperature and humidity test.

B: 1 to 3 points where the foreign matters were observed after the constant temperature and humidity test.

C: 4 to 7 points where the foreign matters were observed after the constant temperature and humidity test.

D: 8 to 15 points where the foreign matters were observed after the constant temperature and humidity test.

E: 16 to 20 points where the foreign matters were observed after the constant temperature and humidity test.

TABLE 16

| | Evaluation 1 | Evaluation 2 |
|---|---|---|
| Example 1 | B | B |
| Example 2 | B | B |
| Example 3 | B | B |
| Example 4 | A | B |
| Example 5 | B | B |
| Example 6 | B | B |
| Example 7 | B | B |
| Example 8 | B | B |
| Example 9 | B | B |
| Example 10 | B | B |
| Example 11 | B | B |
| Example 12 | B | B |
| Example 13 | B | B |
| Example 14 | B | B |
| Example 15 | B | B |
| Example 16 | B | B |
| Example 17 | B | B |
| Example 18 | B | B |
| Example 19 | B | B |
| Example 20 | B | B |
| Example 21 | A | B |
| Example 22 | A | B |
| Example 23 | A | B |
| Example 24 | A | B |
| Example 25 | A | B |
| Example 26 | A | B |
| Example 27 | A | B |
| Example 28 | A | B |
| Example 29 | A | B |
| Example 30 | A | B |
| Example 31 | A | B |
| Example 32 | A | B |
| Example 33 | A | B |
| Example 34 | A | B |
| Example 35 | A | B |
| Example 36 | A | B |
| Example 37 | A | B |
| Example 38 | A | B |
| Example 39 | A | B |
| Example 40 | A | B |
| Example 41 | A | B |
| Example 42 | A | B |
| Example 43 | A | B |
| Example 44 | A | B |
| Example 45 | A | B |
| Example 46 | A | B |
| Example 47 | A | B |
| Example 48 | A | B |
| Example 49 | A | B |
| Example 50 | A | B |
| Example 51 | A | B |
| Example 52 | A | B |
| Example 53 | A | B |
| Example 54 | A | B |
| Example 55 | B | B |
| Example 56 | B | B |
| Example 57 | B | B |
| Example 58 | B | B |
| Example 59 | B | A |
| Example 60 | B | B |
| Example 61 | B | B |
| Example 62 | B | B |
| Example 63 | B | B |
| Example 64 | B | B |
| Example 65 | B | B |
| Example 66 | B | B |
| Example 67 | B | B |
| Example 68 | B | B |
| Example 69 | A | B |
| Example 70 | A | B |
| Example 71 | A | B |
| Example 72 | B | B |
| Example 73 | B | B |
| Example 74 | A | B |
| Example 75 | A | B |
| Example 76 | A | B |
| Example 77 | B | B |
| Example 78 | B | B |
| Example 79 | A | B |
| Example 80 | A | B |
| Example 81 | A | B |
| Example 82 | B | B |
| Example 83 | B | B |
| Example 84 | B | B |
| Example 85 | A | B |
| Example 86 | A | B |
| Example 87 | A | B |
| Example 88 | A | B |
| Example 89 | A | B |
| Example 90 | A | B |
| Example 91 | A | B |
| Example 92 | A | B |
| Example 93 | A | B |
| Example 94 | A | B |
| Example 95 | A | B |
| Example 96 | B | B |
| Example 97 | B | B |
| Example 98 | B | B |
| Example 99 | A | B |
| Example 100 | A | B |
| Example 101 | A | B |
| Example 102 | A | B |
| Example 103 | A | B |
| Example 104 | A | B |
| Example 105 | A | B |
| Example 106 | A | B |
| Example 107 | A | B |
| Example 108 | A | B |
| Example 109 | A | B |
| Example 110 | B | B |
| Example 111 | B | B |
| Example 112 | B | B |
| Example 113 | A | B |
| Example 114 | A | B |
| Example 115 | A | B |
| Example 116 | A | B |
| Example 117 | A | B |
| Example 118 | A | B |
| Example 119 | A | B |
| Example 120 | A | B |

TABLE 17

| | Evaluation 1 | Evaluation |
|---|---|---|
| Example 121 | A | B |
| Example 122 | A | B |
| Example 123 | A | B |
| Example 124 | B | B |
| Example 125 | B | B |
| Example 126 | B | B |
| Example 127 | B | B |
| Example 128 | B | A |
| Example 129 | B | A |
| Example 130 | B | A |
| Example 131 | A | A |
| Example 132 | B | A |
| Example 133 | B | A |
| Example 134 | B | A |
| Example 135 | B | A |
| Example 136 | B | A |
| Example 137 | B | A |
| Example 138 | B | A |
| Example 139 | A | A |
| Example 140 | B | A |
| Example 141 | B | A |
| Example 142 | B | A |
| Example 143 | B | A |
| Example 144 | B | A |
| Example 145 | B | A |
| Example 146 | B | A |
| Example 147 | A | A |
| Example 148 | A | A |
| Example 149 | A | A |
| Example 150 | A | A |
| Example 151 | A | A |
| Example 152 | A | A |
| Example 153 | A | A |
| Example 154 | B | B |
| Example 155 | B | B |
| Example 156 | A | B |
| Example 157 | B | B |
| Example 158 | A | B |
| Example 159 | A | B |
| Example 160 | A | B |
| Example 161 | A | B |
| Example 162 | B | B |
| Example 163 | B | B |
| Example 164 | B | B |
| Example 165 | A | B |
| Example 166 | A | B |
| Example 167 | A | B |
| Example 168 | A | B |
| Example 169 | B | B |
| Example 170 | B | B |
| Example 171 | B | B |
| Example 172 | B | B |
| Example 173 | B | B |
| Example 174 | B | B |
| Example 175 | B | B |
| Comparative example 1 | E | E |
| Comparative example 2 | E | E |
| Comparative example 3 | E | E |
| Comparative example 4 | E | E |
| Comparative example 5 | E | E |
| Comparative example 6 | E | E |
| Comparative example 7 | E | E |
| Comparative example 8 | E | E |
| Comparative example 9 | E | E |
| Comparative example 10 | E | E |
| Comparative example 11 | E | E |
| Comparative example 12 | E | E |
| Comparative example 13 | E | E |
| Comparative example 14 | E | E |

As shown in the tables, with the photosensitive compositions of Examples, a film which had excellent pattern line width stability after leaving and in which generation of defects over time was suppressed could be formed.

What is claimed is:

1. A photosensitive composition comprising:
a coloring material A including a pigment;
a pigment derivative B; and
a dispersant C,
wherein the pigment derivative B includes a pigment derivative B1 in which a maximum value of a molar absorption coefficient in a wavelength range of 400 to 700 nm is 3000 L·mol⁻¹·cm⁻¹ or less,
the dispersant C includes a dispersant C1 having an ethylenically unsaturated bond-containing group, and
a total content of the coloring material A and the pigment derivative B in a total solid content of the photosensitive composition is 50% by mass or more.

2. The photosensitive composition according to claim 1,
wherein the pigment derivative B1 is a compound having a triazine ring.

3. The photosensitive composition according to claim 1,
wherein the pigment derivative B1 is a compound including a group represented by Formula (A1), 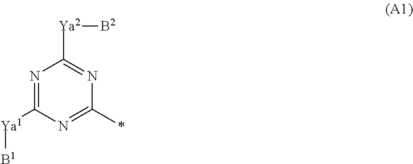
in the formula, * represents a bonding site,
Ya¹ and Ya² each independently represent —N(Ra¹)- or —O—,
Ra¹ represents a hydrogen atom, an alkyl group, an alkenyl group, an alkynyl group, or an aryl group, and
B¹ and B² each independently represent a hydrogen atom or a substituent.

4. The photosensitive composition according to claim 1,
wherein the pigment derivative B includes a pigment derivative B2 in which a maximum value of a molar absorption coefficient in a wavelength range of 400 to 700 nm is more than 3000 L·mol⁻¹·cm⁻¹, and
the pigment derivative B2 is contained in an amount of 10 to 100 parts by mass with respect to 100 parts by mass of the pigment derivative B1.

5. The photosensitive composition according to claim 1,
wherein an ethylenically unsaturated bond-containing group value of the dispersant C1 is 0.01 to 2.0 mmol/g.

6. The photosensitive composition according to claim 1,
wherein the dispersant C contains 30% to 100% by mass of the dispersant C1.

7. The photosensitive composition according to claim 1,
wherein the pigment derivative B is contained in an amount of 3 to 30 parts by mass with respect to 100 parts by mass of a total of the coloring material A and the pigment derivative B.

8. The photosensitive composition according to claim 1,
wherein the dispersant C is contained in an amount of 50 to 1500 parts by mass with respect to 100 parts by mass of the pigment derivative B.

9. The photosensitive composition according to claim 1,
wherein the coloring material A includes a dye.

10. The photosensitive composition according to claim 1, further comprising:
a photopolymerization initiator.

11. The photosensitive composition according to claim 1,
wherein the photosensitive composition is a photosensitive composition for forming a cyan or magenta pixel.

12. A film obtained from the photosensitive composition according to claim 1.

13. An optical filter comprising:
the film according to claim 12.

14. A solid-state imaging element comprising:
the film according to claim 12.

15. An image display device comprising:
the film according to claim 12.
